(19) United States
(12) Patent Application Publication
(10) Pub. No.: US 2011/0027337 A1
(43) Pub. Date: Feb. 3, 2011

(54) PROTEASE INHIBITOR

(75) Inventors: Lehman Kåre Nielsen, Kongerslev (DK); Sondrup Mette Andersen, Aalborg (DK)

Correspondence Address: MERCHANT & GOULD PC, P.O. BOX 2903, MINNEAPOLIS, MN, 55402-0903, US

(73) Assignee: IFXA A/S, Aalborg Ost (DK)

(21) Appl. No.: 12/809,948

(22) PCT Filed: Dec. 19, 2008
(86) PCT No.: PCT/DK08/50334

§ 371 Date: Sep. 23, 2010

Related U.S. Application Data
(60) Provisional application No. 61/009,007, filed on Dec. 21, 2007.

(30) Foreign Application Priority Data
Dec. 21, 2007 (DK) PA 2007 01851

(51) Int. Cl. C07K 14/81; A61K 38/36; A61K 38/57; A61P 9/10; A61P 9/00; A61P 7/00; C12N 5/078; A61F 2/00

(52) U.S. Cl. 424/423; 530/350; 514/21.2; 514/14.2; 514/1.9; 514/14.7; 424/158.1; 435/2

(57) ABSTRACT

The present invention relates to a polypeptide exhibiting a protease inhibitory activity and uses of said polypeptide in methods for inhibiting, directly or indirectly, one or more proteases of the blood clotting cascade. The invention also relates to use of said polypeptide as a pharmaceutical e.g. for prophylactic or ameliorating treatment of blood clots. In addition the invention comprises methods for production of said polypeptide.

### FIELD OF THE INVENTION

The present invention is directed to a polypeptide exhibiting a protease inhibitory activity and uses of said polypeptide in methods for inhibiting, directly or indirectly, one or more proteases.

### TECHNICAL BACKGROUND OF THE INVENTION

The blood coagulation cascade forms part of an important host defense mechanism termed hemostasis (the cessation of blood loss from a damaged vessel). Upon vessel injury, platelets adhere to macromolecules in the subendothelial tissues and then aggregate to form the primary hemostatic plug. The platelets stimulate local activation of plasma coagulation factors, leading to generation of a fibrin clot that reinforces the platelet aggregate. Later, as wound healing occurs, the platelet aggregate and fibrin clot are broken down.

Mechanisms that restrict the above-mentioned formation of platelet aggregates and fibrin clots to sites of injury are necessary to maintain the fluidity of the blood and—in some instances—for preventing undesirable clot formations, such as in thrombosis.

Thrombosis is a pathologic process in which a platelet aggregate and/or a fibrin clot forms in the lumen of an intact blood vessel or in a chamber of the heart. If thrombosis occurs in an artery, the tissue supplied by the artery may undergo ischemic necrosis (e.g., myocardial infarction due to thrombosis of a coronary artery). If thrombosis occurs in a vein, the tissues drained by the vein may become edematous and inflamed. Thrombosis of a deep vein in the lower extremity may be complicated by pulmonary embolism, in which all or a portion of the thrombus breaks loose, is carried in the bloodstream through the vena cava and the right side of the heart, and becomes lodged in a pulmonary artery. Massive pulmonary embolism can cause hypoxemia, shock, and death.

### The Blood Coagulation Cascade

The classical model of blood coagulation involves a series (or “cascade”) of zymogen activation reactions (illustrated in FIG. 1 herein). At each stage, a precursor protein (zymogen) is converted to an active protease by cleavage of one or more peptide bonds in the precursor protein. The types of components that can be involved at each stage include the following:

(a) a protease (from the preceding stage)
(b) a zymogen
(c) a non-enzymatic protein cofactor
(d) calcium ions
(e) an organizing surface (e.g. provided by platelets in vivo)

The blood coagulation or hemostatic system are divided into three parts; platelet aggregation (primary hemostasis), coagulation (secondary hemostasis) and fibrinolysis (tertiary hemostasis). The coagulation protease zymogens involved in hemostasis are procoagulant factors that are secreted by hepatocytes into the bloodstream (prothrombin/factor II, factor VII, factor IX, factor X, factor XI, factor XII and prekallikrein), anticoagulant factors (protein C, protein S) and fibrinolytic factors (plasminogen, t-PA and prourokinase).

Non-enzymatic protein cofactors include factors V and VIII, tissue factor, and high-molecular weight kininogen (HMWK). Factors V and VIII are large plasma proteins that contain repeated sequences homologous to the copper-binding protein ceruloplasmin. Thrombin cleaves V and VIII to yield activated factors (Va and VIIIa) that have at least 50 times the coagulant activity of the precursor forms. Va and VIIIa have no known enzymatic activity. Instead, they serve as cofactors that increase the proteolytic efficiency of Xa and IXa, respectively. Factor VIII circulates in plasma bound to von Willebrand factor (vWF), a glycoprotein that mediates the binding between blood stream platelets and subentothelial structures such as collagen that are exposed upon vessel damage.

The protease factor Xa (fXa) forms part of both the intrinsic pathway and the extrinsic pathway (FIG. 1). These pathways are not redundant but highly interconnected. The coagulation cascade can be initiated in vivo by exposure of plasma to tissue factor. Tissue factor is a non-enzymatic lipoprotein constitutively expressed on the surface of cells that are not normally in contact with plasma (e.g., fibroblasts and macrophages). Exposure of plasma to these cells initiates coagulation outside a broken blood vessel. Endothelial cells also express tissue factor when stimulated by endotoxin, tumor necrosis factor, or interleukin-1, and may be involved in thrombus formation under pathologic conditions.

Tissue factor binds factor VIIa and accelerates factor X activation about 30,000-fold. Although factor VII is activated by its product, the protease factor Xa (fXa), a trace amount of factor VIIa appears to be available in plasma at all times to interact with tissue factor. Factor VIIa also activates factor IX in the presence of tissue factor, providing a connection between the “extrinsic” and “intrinsic” pathways. Factors IXa and Xa assemble with their non-enzymatic protein cofactors (VIIIa and Va, respectively) on the surface of aggregated platelets. This leads to local generation of large amounts of fXa, which activates the final protease generated in the pathway; thrombin (fIIa). Factor Xa converts prothrombin (factor II) to thrombin (factor IIa) by cleaving two peptide bonds in the zymogen. Activation of prothrombin by Xa is accelerated by Va, platelets (or phospholipids), and calcium ions. The complete system activates prothrombin at a rate about 300,000 times greater than that of Xa and calcium alone. Subsequently, thrombin converts the soluble protein fibrinogen into an insoluble fibrin gel, which is strengthened further by covalent cross-linking catalyzed by factor XIIIa.

Fibrinolysis is the process where the enzyme plasmin degrades the formed fibrin clot. Plasmin is produced in an inactive form, plasminogen, in the liver. Although plasminogen cannot cleave fibrin, it still has an affinity for it, and is incorporated into the clot when it is formed. Plasminogen contains secondary structure motifs known as kringles, which bind specifically to lysine and arginine residues on fibrin(ogen). When converted from plasminogen into plasmin it functions as a serine protease, cutting specifically C-terminal to these lysine and arginine residues. Fibrin monomers, when polymerized, form protofibrils. These protofibrils contain two strands, anti-parallel, associated non-covalently. Within a single strand, the fibrin monomers are covalently linked through the actions of coagulation factor XIII. Thus, plasmin action on a clot initially creates nicks in the fibrin; further digestion leads to solubilization.

Tissue plasminogen activator (t-PA) and urokinase are the agents that convert plasminogen to the active plasmin, thus allowing fibrinolysis to occur. t-PA is released into the blood very slowly by the damaged endothelium of the blood vessels, such that after several days (when the bleeding has stopped) the clot is broken down. This occurs because plasminogen became entrapped within the clot when it formed; as it is slowly activated, it breaks down the fibrin mesh. t-PA and urokinase are themselves inhibited by plasminogen activator inhibitor-1 and plasminogen activator inhibitor-2 (PAI-1 and PAI-2). In contrast, plasmin further stimulates plasmin generation by producing more active forms of both tPA and urokinase. Alpha 2-antiplasmin and alpha 2-macroglobulin inactivate plasmin. Plasmin activity is also reduced by thrombin-activatable fibrinolysis inhibitor (TAFI), which modifies fibrin to make a less potent cofactor for the tPA-mediated plasminogen activation.

### Inhibitors of the Blood Coagulation Cascade

The blood coagulation system in held in check by a number of natural occurring inhibitors of the system. Tissue factor pathway inhibitor (TFPI) is a 34-kDa protein associated with plasma lipoproteins and with the vascular endothelium. TFPI is a high affinity serine protease inhibitor containing three Kunitz-type domains, an acidic amino-terminal and an alkaline carboxy-terminal domain. It binds to and inhibits factor Xa. The Xa-TFPI complex then interacts with VIIa/tissue factor and inhibits activation of factors X and IX. TFPI may prevent coagulation unless the VIIa/tissue factor initially present generates a sufficient amount of factor IXa to sustain factor X activation via the “intrinsic” pathway. Thus, VIIa/tissue factor may provide the initial stimulus to clot (in the form of relatively small amounts of IXa and Xa) and then be rapidly turned off, while IXa and VIIIa may be responsible for generating the larger amounts of Xa and thrombin required for clot formation.

Antithrombin is a 58 kDa glycoprotein serine protease inhibitor (serpin) that inactivates the serine proteases; thrombin and fXa, as well as fXIIa, and fIXa. It is constantly active, but its adhesion to these factors is increased by the presence of heparan sulfate proteoglycan (a glycosaminoglycan) or the administration of heparins (different heparinoids increase affinity to F Xa, thrombin, or both). Antithrombin does not inactivate clot-bound thrombin or fXa. Quantitative or qualitative deficiency of antithrombin (inborn or acquired, e.g. in proteinuria) leads to thrombophilia.

Protein C is a major physiological anticoagulant. It is a vitamin K-dependent serine protease enzyme that is activated by thrombin into activated protein C (APC). The activated form (with protein S and phospholipid as a cofactor) degrades Factor Va and Factor VIIIa. The protein C pathway's key enzyme, activated protein C, provides physiologic antithrombotic activity and exhibits both anti-inflammatory and anti-apoptotic activities. Its actions are related to development of thrombosis and ischemic stroke.

Protein S is a vitamin K-dependent plasma glycoprotein synthesized in the liver. In the circulation, Protein S exists in two forms: a free form and a complex form bound to complement protein C4b. The best characterized function of Protein S is its role in the anticoagulation pathway, as it functions as a cofactor to Protein C in the inactivation of Factors Va and VIIIa. Only the free form has cofactor activity. Also, Protein S can bind to negatively charged phospholipids via the carboxylated GLA domain. This property allows Protein S to function in the removal of cells which are undergoing apoptosis, which display negatively charged phospholipids on the cell surface. By binding to the negatively charged phospholipids, Protein S functions as a bridging molecule between the apoptotic cell and the phagocyte.

### Anticoagulants

Under normal conditions, the factors that promote the blood coagulation are in balance with those who inhibit it. Venous or arterial thrombosis occurs when the procoagulant stimuli overwhelm the anticoagulant and fibrinolytic system. Virchow's triad is a group of 3 factors known to affect clot formation: rate of flow, the consistency (thickness) of the blood, and qualities of the vessel wall. Currently, medical intervention for treating thrombosis occur by administering anticoagulants that include parenteral administration of e.g. low-molecular weight heparin (LMWH) followed by oral administration of e.g. warfarin. A newer class of drugs, the direct thrombin inhibitors, is under development; some members are already in clinical use (such as lepirudin). Also under development are other small molecular compounds that interfere directly with the enzymatic action of particular coagulation factors (e.g. rivaroxaban). Anti-platelet agents include aspirin, clopidogrel, dipyridamole and ticlopidine; the parenteral glycoprotein IIb/IIIa inhibitors are used during angioplasty.

Heparin is a naturally occurring highly sulphated glycosaminoglycan produced by basophils and mast cells. Heparin acts as an anticoagulant, preventing the formation of clots and extension of existing clots within the blood. While heparin does not break down clots that have already formed (tissue plasminogen activator will), it allows the body's natural clot lysis mechanisms to work normally to break down clots that have already formed. Heparin binds to the enzyme inhibitor antithrombin III (AT-III) causing a conformational change which results in its active site being exposed. The activated AT-III then inactivates thrombin and other proteases involved in blood clotting, most notably factor Xa. The rate of inactivation of these proteases by AT-III increases 1000-fold due to the binding of heparin. AT-III binds to a specific pentasaccharide sulfation sequence contained within the heparin polymer. The conformational change in AT-III on heparin binding mediates its inhibition of factor Xa. For thrombin inhibition however, thrombin must also bind to the heparin polymer at a site proximal to the pentasaccharide. The highly negative charge density of heparin contributes to its very strong electrostatic interaction with thrombin. The formation of a ternary complex between AT-III, thrombin and heparin results in the inactivation of thrombin. For this reason heparin's activity against thrombin is size dependent, the ternary complex requiring at least 18 saccharide units for efficient formation. In contrast anti factor Xa activity only requires the pentasaccharide binding site. This size difference has led to the development of low molecular weight heparins (LMWHs) and more recently to fondaparinux as pharmaceutical anticoagulants that target anti factor Xa activity rather than anti thrombin (IIa) activity. If long-term anticoagulation is required, heparin is often only used to commence anticoagulation therapy until the oral anticoagulant warfarin takes effect.

Factors II, VII, IX, and X are homologous to each other at their N-terminal ends. After removal of the signal peptide, a carboxylase residing in the endoplasmic reticulum or Golgi binds to the propeptide region of each of these proteins and converts-10-12 glutamate (Glu) residues to g-carboxyglutamate (Gla) in the adjacent “Gla domain”. The propeptide is removed from the carboxylated polypeptide prior to secretion. The Gla residues bind calcium ions and are necessary for the activity of these coagulation factors. Synthesis of Gla requires vitamin K. During g-carboxylation, vitamin K becomes oxidized and must be reduced subsequently in order for the cycle to continue. The anticoagulant drug warfarin (from the group of coumarins) inhibits reduction of vitamin K and thereby prevents synthesis of active factors II, VII, IX, and X.

A serious side-effect of heparin is heparin-induced thrombocytopenia (HIT syndrome). HITS is caused by an immunological reaction that makes platelets aggregate within the blood vessels, thereby using up coagulation factors. Formation of platelet clots can lead to thrombosis, while the loss of coagulation factors and platelets may result in bleeding. HITS can (rarely) occur shortly after heparin is given, but also when a person has been on heparin for a long while. There is also a benign form of thrombocytopenia associated with early heparin use which resolves without stopping heparin. Rarer side effects include alopecia and osteoporosis with chronic use. Also, uncertain responses requires close patient monitoring and allergic responses have been described. As with many drugs, overdoses of heparin can be fatal. With LMWH and fondaparinux, there is a reduced risk of osteoporosis and HIT.

Anticoagulant therapy, usually with heparin injections short term and/or oral anticoagulants (usually warfarin) long term, is clearly effective in prevention of serious vascular events when given as prophylaxis to high-risk patients, or as treatment of acute arterial or venous thrombosis. Anticoagulant therapy thus prevents the formation as well as the extension of existing clots. However, full-dose anticoagulation is also a common cause of major internal bleeding, including intracranial, gastrointestinal or retroperitoneal haemorrhage, which can be fatal. It is therefore important to select patients most likely to benefit from anticoagulant therapy (i.e. those in whom the risk of major thromboembolic events exceeds the risk of major bleeding); and to minimise both thromboembolic and hemorrhagic morbidity and mortality during anticoagulant therapy.

Therefore, there is a need for being able to better control and selectively inhibit the clotting cascade at predetermined stages of the cascade. Developing anticoagulants that act on specific components of the cascade thus increase the efficacy and reduce the side effects administering anticoagulant medicine, such as increased risk of hemorrhaging. The present invention addresses this problem and provides an improved solution for controlling the blood coagulation cascade.

### SUMMARY OF THE INVENTION

The present invention relates to a polypeptide comprising or consisting of SEQ ID NO:1, or a polypeptide having at least 70% sequence identity with SEQ ID NO:1, or a polypeptide fragment of SEQ ID NO:1, said polypeptide or fragment thereof being capable of inhibiting the activity of a protease of the blood clotting cascade.

SEQ ID NO:1 polypeptide sequence:

MMKCLFFLCLCLFPILVFSSTFTSQNPINLPSESPLPKPVLDTNGKELNP
NLSYRIISTYWGALGGDVYLGKSPNSDAPCPDGVFRYNSDVGPSGTPVRF
IPLSTNIFEDQLLNIQFNIPTPKLCVSYTIWKVGNINAPLRTMLLETGGT
IGQADSSYFKIVKSSNFGYNLLYCPITRHFLCPFCRDDNFCAKVGVVIQ
NGKRRLALVNENPLDVLFQEV

The present invention also relates to variants of SEQ ID NO:1 and variants of fragments of SEQ ID NO:1.

The invention further comprises a fusion polypeptide comprising SEQ ID NO:1 operably fused to an N-terminal flanking sequence. Alternatively, fragments of SEQ ID NO:1 are operably fused to an N-terminal flanking sequence. Finally, variants of SEQ ID NO:1 or variants of fragments of SEQ ID NO:1 are operably fused to an N-terminal flanking sequence.

The invention further comprises a fusion polypeptide comprising SEQ ID NO:1 operably fused to an C-terminal flanking sequence. Alternatively, fragments of SEQ ID NO:1 are operably fused to an C-terminal flanking sequence. Finally, variants of SEQ ID NO:1 or variants of fragments of SEQ ID NO:1 are operably fused to an C-terminal flanking sequence.

In another embodiment the present invention relates to an acid addition salt of the polypeptide SEQ ID NO:1, or fragments of SEQ ID NO:1, or variants of SEQ ID NO:1 or variants of fragments of SEQ ID NO:1, said salt preferably being obtainable by treating the polypeptide or fragment or variant thereof with an inorganic acid, such as hydrochloric acid, hydrobromic acid, sulfuric acid, nitric acid, phosphoric acid, and the like, or an organic acid such as an acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, or salicylic acid, to provide a water soluble salt of the polypeptide.

The invention also relates to a method for producing a polypeptide comprising SEQ ID NO:1, or fragments of SEQ ID NO:1, or variants of SEQ ID NO:1 or variants of fragments of SEQ ID NO:1 comprising the steps of collecting potato tuber from Solanum tuberosum and extracting and purifying said polypeptide.

The present invention further comprises methods for the production of a polypeptide comprising SEQ ID NO:1 or fragments of SEQ ID NO:1, as well as methods for the production of variants of SEQ ID NO:1 or the production variants of fragments of SEQ ID NO:1, wherein these methods comprise the steps of providing a polynucleotide or an expression vector comprising any of the above-cited nucleotide sequences and expressing said polynucleotide or said vector in an isolated recombinant or transgenic host cell, thereby producing a polypeptide according to the present invention.

The present invention further comprises methods for the production of proteins with SEQ ID NO:1 or fragments of SEQ ID NO:1, as well as methods for the production of variants of SEQ ID NO:1 or the production variants of fragments of SEQ ID NO:1, wherein these methods comprise the steps of providing a polynucleotide encoding a polypeptide as cited herein above and expressing said polynucleotide either in vitro, or in vivo in a suitable host organism, thereby producing a polypeptide according to the present invention.

In another embodiment the present invention relates to a polynucleotide encoding SEQ ID NO:1, fragments of SEQ ID NO:1, variants of SEQ ID NO:1, or variants of fragments of SEQ ID NO:1. Preferred polynucleotides are e.g. the coding regions of the below listed sequence, with SEQ ID NO:2.

| (SEQ ID NO: 2) | |
|---|---|
| aatcaatatg atgaagtgtt tatttttctt atgtttgtgt ttgtttccca ttttggtgtt | 60 |
| ttcatcaact ttcacttccc aaaatcccat caacctaccc agtgaatctc ctctacctaa | 120 |
| gccggtactt gacacaaatg gtaaagaact caatcctaat ttgagttatc gcattatttc | 180 |
| cacttattgg ggtgccttag gtggtgatgt gtaccttgga aagtccccaa attcagatgc | 240 |
| cccttgtcca gatggcgtat tccgttacaa ttccgatgtt ggacctagcg gtacacccgt | 300 |
| tagattcatt cctttatcta caaatatctt tgaagatcaa ctacttaaca tacaattcaa | 360 |
| tattcctaca ccgaaattat gtgttagtta tacaatttgg aaagtcggta atataaatgc | 420 |
| acctctaagg acgatgttgt tggagactgg aggaaccata gggcaagcag atagcagcta | 480 |
| tttcaagatt gttaaatcat caaattttgg ttacaactta ttgtattgcc ctattactcg | 540 |
| ccattttctt tgtccatttt gtcgtgatga taacttctgt gcaaaagtgg gtgtagttat | 600 |
| tcaaaatgga aaaaggcgtt tggctcttgt caacgaaaat cctcttgatg tcttattcca | 660 |
| ggaagtctag taacaaataa tgcctgcagc tagactatac tatgttttag cctgctggtt | 720 |
| agctactatg ttatgttgta aattaaaata aacacctgct aaggtatatc tatattttag | 780 |
| catggatttc ttaataaatt gtctttcctt atcgtttaaa aaaaaaaaaa aaaaa | 835 |

In one embodiment the invention describes a nucleotide sequence capable of hybridizing to a polynucleotide encoding SEQ ID NO:1, fragments of SEQ ID NO:1, variants of SEQ ID NO:1 or variants of fragments of SEQ ID NO:1.

In another embodiment the invention comprises a polynucleotide encoding SEQ ID NO:1, a polynucleotide encoding a fragment of SEQ ID NO:1, or encoding a variant of SEQ ID NO:1, or encoding a variant of a fragment of SEQ ID NO:1, wherein the portion of said polynucleotide which encodes the polypeptide hybridizes under stringent conditions to a nucleotide probe corresponding to at least 10 consecutive nucleotides of SEQ ID NO:1, or a variant thereof.

The invention also relates to an expression vector comprising a polynucleotide encoding SEQ ID NO:1, fragments of SEQ ID NO:1, variants of SEQ ID NO:1 or variants of fragments of SEQ ID NO:1, said polynucleotide being optionally operably linked to regulatory sequence controlling the expression of said polynucleotide in a suitable host cell.

The invention further relates to an isolated recombinant or transgenic host cell comprising the polypeptide of SEQ ID NO:1, fragments of SEQ ID NO:1, variants of SEQ ID NO:1, or variants of fragments of SEQ ID NO:1.

The invention also relates to a method for generation of a recombinant or transgenic host cell, said method comprising the steps of providing a polynucleotide encoding SEQ ID NO:1, fragments of SEQ ID NO:1, variants of SEQ ID NO:1 or variants of fragments of SEQ ID NO:1, introducing said polynucleotide into said recombinant or transgenic host cell and optionally also expressing said polynucleotide in said recombinant or transgenic host cell, thereby generating a recombinant or transgenic host cell producing said polypeptide.

In another embodiment the present invention relates to a transgenic, mammalian organism comprising the host cell described above, wherein said mammalian host cell is an animal cell selected from the monophyletic group Bilateria, including a mammalian cell belonging to any of the four major lineages Deuterostomes, Ecdysozoa, Platyzoa and Lophotrochozoa.

The mammalian host cell may be an animal cell selected from the group consisting of a Blastomere cell, an Egg cell, an Embryonic stem cell, an Erythrocyte, a Fibroblast, a Hepatocyte, a Myoblast, a Myotube, a Neuron, an Oocyte, an Osteoblast, an Osteoclast, a Sperm cell, a T-Cell and a Zygote.

Also provided is a method for generation of said mammalian host cell, said method comprising the steps of providing a polynucleotide encoding SEQ ID NO:1, fragments of SEQ ID NO:1, variants of SEQ ID NO:1 or variants of fragments of SEQ ID NO:1, introducing said polynucleotide into said recombinant or transgenic host cell and optionally also expressing said polynucleotide in said transgenic, mammalian host cell, thereby generating a transgenic, mammalian host cell producing said polypeptide.

In another embodiment the invention relates to a transgenic plant comprising a recombinant or transgenic host cell comprising the polypeptide of SEQ ID NO:1, fragments of SEQ ID NO:1, variants of SEQ ID NO:1 or variants of fragments of SEQ ID NO:1.

The present invention further relates to a method for generation of the transgenic plant described above, said method comprises the steps of providing a polynucleotide encoding the polypeptide SEQ ID NO:1, fragments of SEQ ID NO:1, variants of SEQ ID NO:1 or variants of fragments of SEQ ID NO:1, introducing said polynucleotide into said plant, thereby generating a transgenic plant producing said polypeptide.

The present invention further relates to a transgenic plant host cell, wherein said host cell is a plant cell of the taxon Embryophyta or Viridiplantae or Chlorobionta, preferably selected from the group consisting of Aleurone cells, Collenchyma cells, Endodermis cells, Endosperm cells, Epidermis cells, Mesophyll cells, Meristematic cells, Palisade cells, Parenchyma cells, Phloem sieve tube cells, Pollen generative cells, Pollen vegetative cells, Sclerenchyma cells, Tracheids cells, Xylem vessel cells and Zygote cells.

In one particular embodiment the transgenic plant is a potato plant.

In another embodiment the invention relates to a recombinant bacterial host cell comprising the polypeptide SEQ ID NO:1, fragments of SEQ ID NO:1, variants of SEQ ID NO:1 or variants of fragments of SEQ ID NO:1 and/or the polynucleotide encoding SEQ ID NO:1, fragments of SEQ ID NO:1, variants of SEQ ID NO:1 or variants of fragments of SEQ ID NO:1 and/or the vector comprising the polynucleotide encoding SEQ ID NO:1, fragments of SEQ ID NO:1, variants of SEQ ID NO:1 or variants of fragments of SEQ ID NO:1.

The invention also relates to a bacterial host cell, wherein said bacterial host cell is selected from a Gram-positive bacterial host cell and a Gram-negative bacterial host cell.

The invention further relates to a method for generating the host cell described above, said method comprising the steps of providing a polynucleotide encoding the polypeptide SEQ ID NO:1, fragments of SEQ ID NO:1, variants of SEQ ID NO:1 or variants of fragments of SEQ ID NO:1, introducing said polynucleotide into said bacterial cell and optionally also expressing said polynucleotide in said bacterial cell, thereby generating a recombinant bacterial cell producing said polypeptide.

In another embodiment the invention relates to a recombinant yeast cell comprising the polypeptide SEQ ID NO:1, fragments of SEQ ID NO:1, variants of SEQ ID NO:1 or variants of fragments of SEQ ID NO:1 and/or the polynucleotide encoding SEQ ID NO:1, fragments of SEQ ID NO:1, variants of SEQ ID NO:1 or variants of fragments of SEQ ID NO:1 and/or the vector comprising the polynucleotide encoding SEQ ID NO:1, fragments of SEQ ID NO:1, variants of SEQ ID NO:1 or variants of fragments of SEQ ID NO:1.

The invention further relates to a method for generating the recombinant yeast cell described above, said method comprising the steps of providing a polynucleotide encoding the polypeptide SEQ ID NO:1, fragments of SEQ ID NO:1, variants of SEQ ID NO:1 or variants of fragments of SEQ ID NO:1, introducing said polynucleotide into said yeast cell and optionally also expressing said polynucleotide in said yeast cell, thereby generating a recombinant yeast cell producing said polypeptide.

In another embodiment the invention relates to a recombinant fungal host cell comprising the polypeptide SEQ ID NO:1, fragments of SEQ ID NO:1, variants of SEQ ID NO:1 or variants of fragments of SEQ ID NO:1 and/or the polynucleotide encoding SEQ ID NO:1, fragments of SEQ ID NO:1, variants of SEQ ID NO:1 or variants of fragments of SEQ ID NO:1 and/or the vector comprising the polynucleotide encoding SEQ ID NO:1, fragments of SEQ ID NO:1, variants of SEQ ID NO:1 or variants of fragments of SEQ ID NO:1.

The invention further relates to a method for generating the recombinant fungal host cell described above, said method comprising the steps of providing a polynucleotide encoding the polypeptide SEQ ID NO:1, fragments of SEQ ID NO:1, variants of SEQ ID NO:1 or variants of fragments of SEQ ID NO:1, introducing said polynucleotide into said fungal cell and optionally also expressing said polynucleotide in said fungal cell, thereby generating a recombinant fungal cell producing said polypeptide.

In another embodiment the present invention relates to an antibody, or a binding fragment thereof, specific for the polypeptide SEQ ID NO:1, fragments of SEQ ID NO:1, variants of SEQ ID NO:1 or variants of fragments of SEQ ID NO:1.

The present invention also relates to a method for generating a polyclonal antibody, or a binding fragment thereof specific for the polypeptide SEQ ID NO:1, fragments of SEQ ID NO:1, variants of SEQ ID NO:1 or variants of fragments of SEQ ID NO:1, said method comprising the steps of immunizing a mammalian subject with said polypeptide under conditions eliciting an antibody response, identifying an antibody which bind specifically to said polypeptide, and optionally isolating said antibody or binding fragment thereof from said mammalian subject.

The present invention further relates to a method for generating a monoclonal antibody specific for the polypeptide SEQ ID NO:1, fragments of SEQ ID NO:1, variants of SEQ ID NO:1 or variants of fragments of SEQ ID NO:1, said method comprising the steps of immunizing a mammalian subject with said polypeptide under conditions eliciting an antibody response, preparing a hybridoma producing a monoclonal antibody specific for said polypeptide, and identifying an antibody which bind specifically to said polypeptide.

The present invention also relates to a polypeptide capable of being recognized by the antibody described above.

In another embodiment the present invention relates to a composition comprising the polypeptide SEQ ID NO:1, fragments of SEQ ID NO:1, variants of SEQ ID NO:1 or variants of fragments of SEQ ID NO:1 in combination with a physiologically acceptable carrier.

The present invention also relates to a pharmaceutical composition comprising the polypeptide SEQ ID NO:1, fragments of SEQ ID NO:1, variants of SEQ ID NO:1 or variants of fragments of SEQ ID NO:1 in combination with a pharmaceutically acceptable carrier.

In another embodiment the present invention relates to a composition comprising the polypeptide SEQ ID NO:1, fragments of SEQ ID NO:1, variants of SEQ ID NO:1 or variants of fragments of SEQ ID NO:1 in combination with a physiologically acceptable carrier and one or more additional bioactive agent(s) acting on platelet aggregation (anti-platelet agent) in hemostasis for medical use.

The present invention also relates to a pharmaceutical composition comprising the polypeptide SEQ ID NO:1, fragments of SEQ ID NO:1, variants of SEQ ID NO:1 or variants of fragments of SEQ ID NO:1 in combination with a pharmaceutically acceptable carrier and one or more additional bioactive agent(s) acting on platelet aggregation (anti-platelet agent) in hemostasis for medical use.

In another embodiment the present invention relates to a composition comprising the polypeptide SEQ ID NO:1, fragments of SEQ ID NO:1, variants of SEQ ID NO:1 or variants of fragments of SEQ ID NO:1 in combination with a physiologically acceptable carrier and one or more additional bioactive agent(s) acting on the blood coagulation cascade (anti-coagulant agent) in hemostasis for medical use.

The present invention also relates to a pharmaceutical composition comprising the polypeptide SEQ ID NO:1, fragments of SEQ ID NO:1, variants of SEQ ID NO:1 or variants of fragments of SEQ ID NO:1 in combination with a pharmaceutically acceptable carrier and one or more additional bioactive agent(s) acting on the blood coagulation cascade (anti-coagulant agent) in hemostasis for medical use.

In another embodiment the present invention relates to a composition comprising the polypeptide SEQ ID NO:1, fragments of SEQ ID NO:1, variants of SEQ ID NO:1 or variants of fragments of SEQ ID NO:1 in combination with a physiologically acceptable carrier and one or more additional bioactive agent(s) acting on fibrinolysis (fibrinolytic agent) in hemostasis for medical use.

The present invention also relates to a pharmaceutical composition comprising the polypeptide SEQ ID NO:1, fragments of SEQ ID NO:1, variants of SEQ ID NO:1 or variants of fragments of SEQ ID NO:1 in combination with a pharmaceutically acceptable carrier and one or more additional bioactive agent(s) acting on fibrinolysis (fibrinolytic agent) in hemostasis for medical use.

In another embodiment the present invention relates to a composition comprising the polypeptide SEQ ID NO:1, fragments of SEQ ID NO:1, variants of SEQ ID NO:1 or variants of fragments of SEQ ID NO:1 in combination with a physiologically acceptable carrier and one or more additional bioactive agent(s) selected from the group of anti-platelet, anti-coagulation and fibrinolytic agent(s) for medical use.

The present invention also relates to a pharmaceutical composition comprising the polypeptide SEQ ID NO:1, fragments of SEQ ID NO:1, variants of SEQ ID NO:1 or variants of fragments of SEQ ID NO:1 in combination with a pharmaceutically acceptable carrier and one or more additional bioactive agent(s) selected from the group of anti-platelet, anti-coagulation and fibrinolytic agent(s) for medical use.

In yet another embodiment the present invention relates to a kit-of-parts comprising the polypeptide SEQ ID NO:1, fragments of SEQ ID NO:1, variants of SEQ ID NO:1 or variants of fragments of SEQ ID NO:1 or one or the compositions described above, and at least one additional component.

The present invention also relates to a method for identifying binding partners for the polypeptide SEQ ID NO:1, fragments of SEQ ID NO:1, variants of SEQ ID NO:1 or variants of fragments of SEQ ID NO:1, said method comprising the steps of extracting said polypeptide and isolating said binding partners.

The present invention relates to a polypeptide SEQ ID NO:1, fragments of SEQ ID NO:1, variants of SEQ ID NO:1 or variants of fragments of SEQ ID NO:1 or one or the composition comprising SEQ ID NO:1, fragments of SEQ ID NO:1, variants of SEQ ID NO:1 or variants of fragments of SEQ ID NO:1 described above for use as a medicament.

The present invention also relates to a method for treatment of an individual in need thereof with the binding partners described above such as agonists or antagonists of SEQ ID NO:1, fragments of SEQ ID NO:1, variants of SEQ ID NO:1 or variants of fragments of SEQ ID NO:1.

The present invention also relates to a method for treatment of an individual in need thereof with the binding partners described above such as agonists or antagonists of SEQ ID NO:1, fragments of SEQ ID NO:1, variants of SEQ ID NO:1 or variants of fragments of SEQ ID NO:1 in combination with the polypeptide The present invention also relates to a method for treatment of an individual in need thereof with the binding partners described above such as agonists or antagonists of SEQ ID NO:1, fragments of SEQ ID NO:1, variants of SEQ ID NO:1 or variants of fragments of SEQ ID NO:1.

The present invention further relates to a method for treatment of coronary syndromes comprising administration of one of the compositions described above to an individual in need thereof.

In another embodiment the present invention relates to a pharmaceutical composition for treating coronary syndromes selected from the group consisting of: stable angina pectoris, unstable angina pectoris, myocardial ischemia, myocardial infarction, dilated cardiomyopathy, hypertropic cardiomyopathy, congestive heart failure and cardiac failure, comprising one or more of the composition described above.

In addition the present invention relates to a method for treatment of atrial fibrillation, comprising administration of one or more of the compositions described above to an individual in need thereof.

The present invention also relates to a pharmaceutical composition for treating atrial fibrillation and cardioversion comprising one or more of the composition described above.

The present invention further relates to a method for treatment of peripheral arterial occlusion comprising administration of one or more of the compositions described above to an individual in need thereof.

In another embodiment the invention relates to a pharmaceutical composition for treating peripheral arterial occlusion caused by primary or recurrent thrombus formation, embolism or atherosclerosis, comprising one or more of the compositions described above.

The present invention also relates to a method for treatment of deep-vein thrombosis comprising administration of one or more of the compositions described above to an individual in need thereof.

In another embodiment the invention relates to a pharmaceutical composition for treating deep-vein thrombosis and pulmonary embolism comprising one or more of the compositions described above.

The present invention also relates to a method for treatment of blood clotting in extracorporal circuits and catheters, comprising administration of one or more of the compositions described above to an individual in need thereof.

In another embodiment the invention relates to a pharmaceutical composition for treating blood clotting in extracorporal circuits during cardiopulmonary bypass and hemodialysis comprising one or more of the compositions described above.

The present invention further relates to a method for treatment of blood clotting during angioplastic procedures, comprising administration of one or more of the compositions described above to an individual in need thereof.

In another embodiment the present invention relates to a pharmaceutical composition for treating blood clotting during angioplastic procedures comprising one or moe of the compositions described above.

The present invention also relates to a method for treatment of blood clotting in connection with artificial heart valve replacement, comprising administration of one or more of the compositions described above to an individual in need thereof.

In another embodiment the invention relates to a pharmaceutical composition for treating blood clotting in connection with prosthetic heart valve replacement comprising one or more of the compositions described above.

The present invention further relates to a method for treatment of blood clotting due to diseases affecting the heart valves, comprising administration of one or more of the compositions described above.

In another embodiment the present invention relates to a pharmaceutical composition for treating blood clotting due to diseases affecting the heart valves comprising infected valves (bacterial endocarditis), rheumatic mitral valve disease, mitral stenosis, mitral valve prolapse, mitral annular calcification and isolated aortic valve disease comprising one or more of the compositions described above.

The present invention also relates to a method for treatment of blood clotting in patients with thrombophilia syndromes, comprising administration of one or more of the compositions described above.

In one preferred embodiment the present invention relates to a pharmaceutical composition for treating blood clotting in patients with thrombophilia syndromes including antiphospholipid syndrome, Factor V Leiden, prothrombin mutation/factor II mutation, high homocysteine levels due to MTHFR mutation or vitamin deficiency (vitamins B6, B12 and folic acid), renal loss of antithrombin, plasminogen and fibrinolysis disorders, paroxysmal nocturnal hemoglobinuria, protein C deficiency, protein S deficiency, and antithrombin III deficiency comprising one or more of the compositions described above.

The invention also relates to a method for treatment of blood clotting following coagulation management, comprising administration of one or more of the compositions described above.

In yet another embodiment the invention relates to a pharmaceutical composition for treating blood clotting following coagulation management warranted by the presence of conditions that increase the risk of bleeding comprising Hemophilia A, Hemophilia B, Hemophilia C, Von Willebrand disease, major blood loss, Glanzmann's thrombasthenia, Bernard-Soulier syndrome, gray platelet syndrome and delta storage pool deficiency, comprising one or more of the composition described above.

In addition the present invention relates to a method for treatment of decreased platelet numbers leading to increased platelet activation comprising administration of one or more of the compositions described above to an individual in need thereof.

In another preferred embodiment the invention relates to a pharmaceutical composition for treating blood clotting due to decreased platelet number caused by insufficient production (e.g. in myelodysplastic syndrome or other bone marrow disorders), destruction by the immune system (immune thrombocytopenic purpura/ITP), and consumption due to various causes (thrombotic thrombocytopenic purpura/TTP, hemolytic-uremic syndrome/HUS, paroxysmal nocturnal hemoglobinuria/PNH, disseminated intravascular coagulation/DIC, heparin-induced thrombocytopenia/HIT), comprising one or more of the composition described above.

The present invention further relates to a method for treatment of blood clotting for any reason in individuals who do not tolerate other medicaments targeting blood clotting on the market, comprising administration of one or more of the compositions described above to an individual in need thereof.

In another preferred embodiment the present invention relates to a pharmaceutical composition for treating blood clotting in individuals who do not tolerate other medicaments targeting blood clotting on the market comprising one or more of the compositions described above.

The present invention also relates to a method for treatment of blood clotting in individuals that are immobilized for any reason, patients that suffer from critical limb ischemia or have had a limb amputated, comprising administration of one or more of the compositions described above to an individual in need thereof.

In another embodiment the invention relates to a pharmaceutical composition for treating blood clotting in individuals that are immobilized for any reason, patients that suffer from critical limb ischemia or have had a limb amputated, comprising one or more of the compositions described above.

The present invention also relates to a method for treatment of blood clotting in patients receiving any form for chemotherapeutics, comprising administration of one or more of the composition described above to an individual in need thereof.

In another preferred embodiment the invention relates to a pharmaceutical composition for treating blood clotting in patients receiving any form for chemotherapeutics, comprising one or more of the compositions described above.

The present invention further relates to a method for treatment of blood clotting in patients at high risk for developing blood clots, comprising administration of one or more of the compositions described above to an individual in need thereof.

In yet another preferred embodiment the present invention relates to a pharmaceutical composition for treating blood clotting in patients at high risk for developing blood clots, comprising one or more of the compositions described above.

### DEFINITIONS

“Thrombosis” refers to thrombus formation, and a “thrombus” is a blood clot i.e. the final step in the blood coagulation cascade of hemostasis. A thrombus is physiologic in cases of injury, but pathologic in case of thrombosis thus occurring in an intact blood vessel. Thrombosis usually occurs at sites of vessel or heart wall damage (e.g. rupture of an atherosclerotic plaque; diseased or replacement heart valves; mural thrombus following endocardial injury after myocardial infarction) and/or blood flow disturbance (e.g. atrial fibrillation, leg veins in immobile patients). The contributions of platelet-fibrin thrombi to arterial and venous occlusion and to clinical events have been established by morphological studies (pathological, angiographic and angioscopic); epidemiological studies of haemostatic variables (related to platelets, coagulation and fibrinolysis); and especially by large randomised controlled trials of antiplatelet, anticoagulant or thrombolytic therapies.

An “embolism” occurs when an object (the embolus, plural emboli) migrates from one part of the body (through circulation) and cause(s) a blockage (occlusion) of a blood vessel in another part of the body.

“Hemostasis” is a term that refer to the physiologic process whereby bleeding is halted. It consists of multiple steps including 1) vasoconstriction to minimize vessel lumen diameter and slow bleeding, 2) platelet aggregation, 3) coagulation and 4) fibrinolysis whereby the blood clot is degraded.

The term “blood clotting cascade” or “blood coagulation cascade” is part of secondary hemostasis and refers to the multi-step process whereby blood and vessel components react to stimuli by the enzymatic activation of coagulation factors sequentially, ultimately resulting in the formation of a solid blood clot comprising fibrin gel and platelets.

The term “cardioversion” is the process whereby an abnormally fast heart rate or cardiac arrhythmia is terminated. This can be obtained either by the delivery of a therapeutic dose of electrical current to the heart at a specific moment in the cardiac cycle (denoted ‘synchronized electrical cardioversion’), or by using medication instead of an electrical shock to convert the cardiac arrhythmia ('pharmacologic cardioversion').

A “Bioactive agent” is any agent, drug, compound, composition of matter or mixture which provides some pharmacologic, often beneficial, effect that can be demonstrated in-vivo or in vitro. As used herein, this term further includes any physiologically or pharmacologically active substance that produces a localized or systemic effect in a patient. Further examples of bioactive agents include, but are not limited to, agents comprising or consisting of an oligosaccharide, agents comprising or consisting of a polysaccharide, agents comprising or consisting of an optionally glycosylated peptide, agents comprising or consisting of an optionally glycosylated polypeptide, agents comprising or consisting of an oligonucleotide, agents comprising or consisting of a polynucleotide, agents comprising or consisting of a lipid, agents comprising or consisting of a fatty acid, agents comprising or consisting of a fatty acid ester and agents comprising or consisting of secondary metabolites.

The terms “treating”, “treatment” and “therapy” as used herein refer equally to curative therapy, prophylactic or preventative therapy and ameliorating therapy. The term includes an approach for obtaining beneficial or desired physiological results, which may be established clinically. For purposes of this invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering compositions of the present invention.

The term “secondary prophylaxis” refer to prophylactic therapy after the first occurrence of a pathological condition, such as myocardial infarction, ischemic stroke, angina pectoris and peripheral arterial disease.

A “treatment effect” or “therapeutic effect” is manifested if there is a change in the condition being treated, as measured by the criteria constituting the definition of the terms “treating” and “treatment.” There is a “change” in the condition being treated if there is at least 5% improvement, preferably 10% improvement, more preferably at least 25%, even more preferably at least 50%, such as at least 75%, and most preferably at least 100% improvement. The change can be based on improvements in the severity of the treated condition in an individual, or on a difference in the frequency of improved conditions in populations of individuals with and without treatment with the bioactive agent, or with the bioactive agent in combination with a pharmaceutical composition of the present invention.

“Pharmacologically effective amount”, “pharmaceutically effective amount” or “physiologically effective amount of a “bioactive agent” is the amount of an active agent present in a pharmaceutical composition as described herein that is needed to provide a desired level of active agent in the bloodstream or at the site of action in an individual (e.g., the lungs, the gastric system, the colorectal system, prostate, etc.) to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of a bioactive agent can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

The terms “enhancing” and “improving” a beneficial effect, and variations thereof, as used herein, refers to the therapeutic effect of the bioactive agent against placebo, or an increase in the therapeutic effect of a state-of-the-art medical treatment above that normally obtained when a pharmaceutical composition is administered without the bioactive agent of this invention. “An increase in the therapeutic effects” is manifested when there is an acceleration and/or increase in intensity and/or extent of the therapeutic effects obtained as a result of administering the bioactive agent(s). It also includes extension of the longevity of therapeutic benefits. It can also manifest where a lower amount of the pharmaceutical composition is required to obtain the same benefits and/or effects when it is co-administered with bioactive agent(s) provided by the present invention as compared to the administration in a higher amount of the pharmaceutical composition in the absence of bioactive agent. The enhancing effect preferably, but not necessarily, results in treatment of acute symptoms for which the pharmaceutical composition alone is not effective or is less effective therapeutically. Enhancement is achieved when there is at least a 5% increase in the therapeutic effects, such as at least 10% increase in the therapeutic effects when a bioactive agent of the present invention is co-administered with a pharmaceutical composition compared with administration of the pharmaceutical composition alone. Preferably the increase is at least 25%, more preferably at least 50%, even more preferably at least 75%, most preferably at least 100%.

“Co-administering” or “co-administration” of bioactive agent(s), or bioactive agents and state-of-the-art medicaments, as used herein, refers to the administration of one or more bioactive agents of the present invention, or administration of one or more bioactive agents of the present invention and a state-of-the-art pharmaceutical composition within a certain time period. The time period is preferably less than 72 hours, such as 48 hours, for example less than 24 hours, such as less than 12 hours, for example less than 6 hours, such as less than 3 hours. However, these terms also mean that the bioactive agent and a therapeutic composition can be administered together.

The term “Individual” refers to vertebrates, particular members of the mammalian species, and includes, but is not limited to domestic animals, such as cattle, horses, pigs, sheep, mink, dogs, cats, mice, guinea pigs, rabbits, rats; sports animals, such as horses, poly ponies, dogs, camels, and primates, including humans.

The term “Kit of parts” as used in the present invention provides the polypeptide according to the present invention and a second bioactive agent for administration in combination. The combined active substances may be used for simultaneous, sequential or separate administration. In all cases, it is preferred that any of the herein-mentioned medicaments and bioactive agents are administered in pharmaceutically effective amounts, i.e. an administration involving a total amount of each active component of the medicament or pharmaceutical composition or method that is sufficient to show a meaningful patient benefit. The formulations may conveniently be presented in unit dosage form by methods known to those skilled in the art. It is preferred that the kit may for example contain the active compounds in dosage forms for administration. A dosage form contains a sufficient amount of one or more of the active compound(s) such that a desirable effect can be obtained when administered to a subject. Thus, it is preferred that the medical packaging comprises an amount of dosage units corresponding to the relevant dosage regimen. Accordingly, in one embodiment, the medical packaging comprises a pharmaceutical composition comprising the compounds as defined above or a pharmaceutically acceptable salt thereof and pharmaceutically acceptable carriers, vehicles and/or excipients. The medical packaging may be in any suitable form—for example for enteral (via the digestive tract) or parenteral (routes other than the digestive tract) administration. In another preferred embodiment the packaging is in the form of a cartridge, such as a cartridge for an injection pen, the injection pen being such as an injection pen known from insulin treatment. Preferably, the kit-of-parts contains instructions indicating the use of the dosage form to achieve a desirable affect and the amount of dosage form to be taken over a specified time period. Accordingly, in one embodiment the medical packaging comprises instructions for administering the pharmaceutical composition. It is envisaged that at least one (such as 2 or 3) additional medicament(s) acting on hemostasis or on treatment on the underlying cause of hemostasis or risk hereof, and at least one (such as 2 or 3) polypeptide according to the present invention may be used for the manufacture of any of the “kit of parts” described herein for administration to an individual in need thereof.

A “Bioprosthetic valve” is an artificial or prosthetic heart valve comprising biological materiel. Such biological or bioprosthetic valves are valves of animals, like pigs, which undergo several chemical procedures in order to make them suitable for implantation in the human heart. The porcine (or pig) heart is most similar to the human heart, and therefore represents the best anatomical fit for replacement. Implantation of a porcine valve is a type of xenotransplantation, or xenograft, which means a transplant from one species (in this case a pig) to another. There are some risks associated with a Xenograft such as the human body's tendency to reject foreign material. Medication can be used to retard this effect, but is not always successful. Another type of biological valve utilizes biological tissue to make leaflets that are sewn into a metal frame. This tissue is typically harvested from the pericardial sac of either bovine (cows) or equine (horses). The pericardial sac is particularly well suited for a valve leaflet due to its extremely durable physical properties. This type of biological valve is an extremely effective means of valve replacement. The tissue is sterilized so that the biological markers are removed, eliminating a response from the host's immune system. The most used heart valves in the US and EU are those utilizing tissue leaflets. Mechanical valves are more commonly used in Asia and Latin America.

The term “homolog to SEQ ID NO:1” refers to a polypeptide with a sequence similar to but unlike SEQ ID NO:1, in that it is a polypeptide comprising or consisting of SEQ ID NO:1 or a fragment hereof.

The term “variant of SEQ ID NO:1” refers to a polypeptide with a sequence similar to but unlike SEQ ID NO:1, in that it is a polypeptide variant of SEQ ID NO:1 or a fragment hereof. The variant polypeptide has an amino acid sequence that is a modification of the polypeptide according to the present invention. The modification includes one or more conservative substitution(s) or one or more equivalent substitution(s) of one or more amino acids that alters the sequence, but not the biological activity, of the polypeptide of SEQ ID NO:1.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., (alpha-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “natural nucleotide” refers to any of the four deoxyribonucleotides, dA, dG, dT, and dC (constituents of DNA), and the four ribonucleotides, A, G, U, and C (constituents of RNA) are the natural nucleotides. Each natural nucleotide comprises or essentially consists of a sugar moiety (ribose or deoxyribose), a phosphate moiety, and a natural/standard base moiety. Natural nucleotides bind to complementary nucleotides according to well-known rules of base pairing (Watson and Crick), where adenine (A) pairs with thymine (T) or uracil (U); and where guanine (G) pairs with cytosine (C), wherein corresponding base-pairs are part of complementary, anti-parallel nucleotide strands. The base pairing results in a specific hybridization between predetermined and complementary nucleotides. The base pairing is the basis by which enzymes are able to catalyze the synthesis of an oligonucleotide complementary to the template oligonucleotide. In this synthesis, building blocks (normally the triphosphates of ribo or deoxyribo derivatives of A, T, U, C, or G) are directed by a template oligonucleotide to form a complementary oligonucleotide with the correct, complementary sequence. The recognition of an oligonucleotide sequence by its complementary sequence is mediated by corresponding and interacting bases forming base pairs. In nature, the specific interactions leading to base pairing are governed by the size of the bases and the pattern of hydrogen bond donors and acceptors of the bases. A large purine base (A or G) pairs with a small pyrimidine base (T, U or C). Additionally, base pair recognition between bases is influenced by hydrogen bonds formed between the bases. In the geometry of the Watson-Crick base pair, a six membered ring (a pyrimidine in natural oligonucleotides) is juxtaposed to a ring system composed of a fused, six membered ring and a five membered ring (a purine in natural oligonucleotides), with a middle hydrogen bond linking two ring atoms, and hydrogen bonds on either side joining functional groups appended to each of the rings, with donor groups paired with acceptor groups.

The term “complement of a nucleic acid molecule” refers to a nucleic acid molecule having a complementary nucleotide sequence and reverse orientation as compared to a reference nucleotide sequence. For example, the sequence 5′ ATGCACGGG 3′ is complementary to 5′ CCCGTGCAT 3′.

The term “degenerate nucleotide sequence” denotes a sequence of nucleotides that includes one or more degenerate codons as compared to a reference nucleic acid molecule that encodes a polypeptide. Degenerate codons contain different triplets of nucleotides, but encode the same amino acid residue (i.e., GAU and GAC triplets each encode Asp).

The term “structural gene” refers to a nucleic acid molecule that is transcribed into messenger RNA (mRNA), which is then translated into a sequence of amino acids characteristic of a specific polypeptide.

An “isolated nucleic acid molecule” is a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

A “nucleic acid molecule construct” is a nucleic acid molecule, either single- or double-stranded, that has been modified through human intervention to contain segments of nucleic acid combined and juxtaposed in an arrangement not existing in nature.

“Linear DNA” denotes non-circular DNA molecules having free 5′ and 3′ ends. Linear DNA can be prepared from closed circular DNA molecules, such as plasmids, by enzymatic digestion or physical disruption.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (GREs), serum response elements (SREs; Treisman, Seminars in Cancer Biol. 1:47 (1990)), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the TATA box and start of transcription. By this definition, a core promoter may or may not have detectable activity in the absence of specific sequences that may enhance the activity or confer tissue specific activity.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner.

An “enhancer” is a type of regulatory element that can increase the efficiency of transcription, regardless of the distance or orientation of the enhancer relative to the start site of transcription.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

A “polypeptide” is a polymer of amino acid residues preferably joined exclusively by peptide bonds, whether produced naturally or synthetically. A polypeptide produced by expression of a non-host DNA molecule is a “heterologous” peptide or polypeptide. The term “polypeptide” as used herein covers proteins, peptides and polypeptides, wherein said proteins, peptides or polypeptides may or may not have been post-translationally modified. Post-translational modification may for example be phosphorylation, methylation and glucosylation.

An “amino acid residue” can be a natural or non-natural amino acid residue linked peptide bonds or bonds different from peptide bonds. The amino acid residues can be in D-configuration or L-configuration. An amino acid residue comprises an amino terminal part (NH₂) and a carboxy terminal part (COOH) separated by a central part comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822(b)(2) belong to the group of amino acids listed in Table 1 herein below. Non-natural amino acids are those not listed in Table 1. Examples of non-natural amino acids are those listed e.g. in 37 C.F.R. section 1.822(b)(4), all of which are incorporated herein by reference. Also, non-natural amino acid residues include, but are not limited to, modified amino acid residues, L-amino acid residues, and stereoisomers of D-amino acid residues.

TABLE 1
Natural amino acids and their respective codes.

Symbols

| 1-Letter | 3-Letter | Amino acid |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”. Preferred “equivalent amino acid residues” of residues of SEQ ID NO:1 are listed herein below in Table 2: equivalent amino acid residue(s).

The classification of equivalent amino acids refers in one embodiment to the following classes: 1) HRK, 2) DENQ, 3) C, 4) STPAG, 5) MILV and 6) FYW

Within the meaning of the term “equivalent amino acid substitution” as applied herein, one amino acid may be substituted for another, in one embodiment, within the groups of amino acids indicated herein below:
i) Amino acids having polar side chains (Asp, Glu, Lys, Arg, His, Asn, Gln, Ser, Thr, Tyr, and Cys,)
ii) Amino acids having non-polar side chains (Gly, Ala, Val, Leu, Ile, Phe, Trp, Pro, and Met)
iii) Amino acids having aliphatic side chains (Gly, Ala Val, Leu, Ile)
iv) Amino acids having cyclic side chains (Phe, Tyr, Trp, His, Pro)
v) Amino acids having aromatic side chains (Phe, Tyr, Trp)
vi) Amino acids having acidic side chains (Asp, Glu)
vii) Amino acids having basic side chains (Lys, Arg, His)
viii) Amino acids having amide side chains (Asn, Gln)
ix) Amino acids having hydroxy side chains (Ser, Thr)
x) Amino acids having sulphor-containing side chains (Cys, Met),
xi) Neutral, weakly hydrophobic amino acids (Pro, Ala, Gly, Ser, Thr)
xii) Hydrophilic, acidic amino acids (Gln, Asn, Glu, Asp), and
xiii) Hydrophobic amino acids (Leu, Ile, Val)

A Venn diagram is another method for grouping of amino acids according to their properties (Livingstone & Barton, CABIOS, 9, 745-756, 1993). In another preferred embodiment one or more amino acids may be substituted with another within the same Venn diagram group.

The present invention also relates to variants of the polypeptides SEQ ID NO:1, or fragments thereof, wherein the substitutions have been designed by computational analysis that uses sequence homology to predict whether a substitution affects protein function (e.g. Pauline C. Ng and Steven Henikoff, Genome Research, Vol. 11, Issue 5, 863-874, May 2001).

TABLE 2
Preferred, equivalent amino acid residue(s)
in SEQ ID NO: 1.

The aa-sequence of SEQ ID NO: 1 of the present
invention contains 220 amino acid residues
in the following order:
MMKCLFFLCLCLFPILVFSSTFTSQNPINLPSESPLPKPVLDTNGKELNP
NLSYRIISTYWGALGGDVYLGKSPNSDAPCPDGVFRYNSDVGPSGTPV
RFIPLSTNIFEDQLLNIQFNIPTPKLCVSYTIWKVGNINAPLRTMLLETG
GTIGQADSSYFKIVKSSNFGYNLLYCPITRHFLCPFCRDDNFCAKVGVV
IQNGKRRLALVNENPLDVLFQEV (SEQ ID NO: 1)

Below is represented, with an X, non-limiting
examples of what is meant by an equivalent
amino acid substitution for each amino acid
at each position:

| | Y | G | F | M | A | S | I | L | T | V | P | K | H | Q | E | W | R | D | N | C |
|---|---|---|---|---|---|---|---|---|---|---|---|---|---|---|---|---|---|---|---|---|
| 1-M | | | | | | | X | X | | X | | | | | | | | | | |
| 2-M | | | | | | | X | X | | X | | | | | | | | | | |
| 3-K | | | | | | | | | | | | | X | | | | X | | | |
| 4-C | | | | | | | | | | | | | | | | | | | | |
| 5-L | | | | X | | | X | | | X | | | | | | | | | | |
| 6-F | X | | | | | | | | | | | | | | | X | | | | |
| 7-F | X | | | | | | | | | | | | | | | X | | | | |
| 8-L | | | | X | | | X | | | X | | | | | | | | | | |
| 9-C | | | | | | | | | | | | | | | | | | | | |
| 10-L | | | | X | | | X | | | X | | | | | | | | | | |
| 11-C | | | | | | | | | | | | | | | | | | | | |
| 12-L | | | | X | | | X | | | X | | | | | | | | | | |
| 13-F | X | | | | | | | | | | | | | | | X | | | | |
| 14-P | | X | | | X | X | | | X | | | | | | | | | | | |
| 15-I | | | | X | | | | X | | X | | | | | | | | | | |
| 16-L | | | | X | | | X | | | X | | | | | | | | | | |
| 17-V | | | | X | | | X | X | | | | | | | | | | | | |
| 18-F | X | | | | | | | | | | | | | | | X | | | | |
| 19-S | | X | | | X | | | | X | | X | | | | | | | | | |
| 20-S | | X | | | X | | | | X | | X | | | | | | | | | |
| 21-T | | X | | | X | X | | | | | X | | | | | | | | | |
| 22-F | X | | | | | | | | | | | | | | | X | | | | |
| 23-T | | X | | | X | X | | | | | X | | | | | | | | | |
| 24-S | | X | | | X | | | | X | | X | | | | | | | | | |
| 25-Q | | | | | | | | | | | | | | | X | | | X | X | |
| 26-N | | | | | | | | | | | | | | X | X | | | X | | |
| 27-P | | X | | | X | X | | | X | | | | | | | | | | | |
| 28-I | | | | X | | | | X | | X | | | | | | | | | | |
| 29-N | | | | | | | | | | | | | | X | X | | | X | | |
| 30-L | | | | X | | | X | | | X | | | | | | | | | | |
| 31-P | | X | | | X | X | | | X | | | | | | | | | | | |
| 32-S | | X | | | X | | | | X | | X | | | | | | | | | |
| 33-E | | | | | | | | | | | | | | X | | | | X | X | |
| 34-S | | X | | | X | | | | X | | X | | | | | | | | | |
| 35-P | | X | | | X | X | | | X | | | | | | | | | | | |
| 36-L | | | | X | | | X | | | X | | | | | | | | | | |
| 37-P | | X | | | X | X | | | X | | | | | | | | | | | |
| 38-K | | | | | | | | | | | | | X | | | | X | | | |
| 39-P | | X | | | X | X | | | X | | | | | | | | | | | |
| 40-V | | | | X | | | X | X | | | | | | | | | | | | |
| 41-L | | | | X | | | X | | | X | | | | | | | | | | |
| 42-D | | | | | | | | | | | | | | X | X | | | | X | |
| 43-T | | X | | | X | X | | | | | X | | | | | | | | | |
| 44-N | | | | | | | | | | | | | | X | X | | | X | | |
| 45-G | | | | | X | X | | | X | | X | | | | | | | | | |
| 46-K | | | | | | | | | | | | | X | | | | X | | | |
| 47-E | | | | | | | | | | | | | | X | | | | X | X | |
| 48-L | | | | X | | | X | | | X | | | | | | | | | | |
| 49-N | | | | | | | | | | | | | | X | X | | | X | | |
| 50-P | | X | | | X | X | | | X | | | | | | | | | | | |
| 51-N | | | | | | | | | | | | | | X | X | | | X | | |
| 52-L | | | | X | | | X | | | X | | | | | | | | | | |
| 53-S | | X | | | X | | | | X | | X | | | | | | | | | |
| 54-Y | | | X | | | | | | | | | | | | | X | | | | |
| 55-R | | | | | | | | | | | | X | X | | | | | | | |
| 56-I | | | | X | | | | X | | X | | | | | | | | | | |
| 57-I | | | | X | | | | X | | X | | | | | | | | | | |
| 58-S | | X | | | X | | | | X | | X | | | | | | | | | |
| 59-T | | X | | | X | X | | | | | X | | | | | | | | | |
| 60-Y | | | X | | | | | | | | | | | | | X | | | | |
| 61-W | X | | X | | | | | | | | | | | | | | | | | |
| 62-G | | | | | X | X | | | X | | X | | | | | | | | | |
| 63-A | | X | | | | X | | | X | | X | | | | | | | | | |
| 64-L | | | | X | | | X | | | X | | | | | | | | | | |
| 65-G | | | | | X | X | | | X | | X | | | | | | | | | |
| 66-G | | | | | X | X | | | X | | X | | | | | | | | | |
| 67-D | | | | | | | | | | | | | | X | X | | | | X | |
| 68-V | | | | X | | | X | X | | | | | | | | | | | | |
| 69-Y | | | X | | | | | | | | | | | | | | | | | |
| 70-L | | | | X | | | X | | | X | | | | | | | | | | |
| 71-G | | | | | X | X | | | X | | X | | | | | | | | | |
| 72-K | | | | | | | | | | | | | X | | | | X | | | |
| 73-S | | X | | | X | | | | X | | X | | | | | | | | | |
| 74-P | | X | | | X | X | | | X | | | | | | | | | | | |
| 75-N | | | | | | | | | | | | | | X | X | | | X | | |
| 76-S | | X | | | X | | | | X | | X | | | | | | | | | |
| 77-D | | | | | | | | | | | | | | X | X | | | | X | |
| 78-A | | X | | | | X | | | X | | X | | | | | | | | | |
| 79-P | | X | | | X | X | | | X | | | | | | | | | | | |
| 80-C | | | | | | | | | | | | | | | | | | | | |
| 81-P | | X | | | X | X | | | X | | | | | | | | | | | |
| 82-D | | | | | | | | | | | | | | X | X | | | | X | |
| 83-G | | | | | X | X | | | X | | X | | | | | | | | | |
| 84-V | | | | X | | | X | X | | | | | | | | | | | | |
| 85-F | X | | | | | | | | | | | | | | | X | | | | |
| 86-R | | | | | | | | | | | | X | X | | | | | | | |
| 87-Y | | | X | | | | | | | | | | | | | X | | | | |
| 88-N | | | | | | | | | | | | | | X | X | | | X | | |
| 89-S | | X | | | X | | | | X | | X | | | | | | | | | |
| 90-D | | | | | | | | | | | | | | X | X | | | | X | |
| 91-V | | | | X | | | X | X | | | | | | | | | | | | |
| 92-G | | | | | X | X | | | X | | X | | | | | | | | | |
| 93-P | | X | | | X | X | | | X | | | | | | | | | | | |
| 94-S | | X | | | X | | | | X | | X | | | | | | | | | |
| 95-G | | | | | X | X | | | X | | X | | | | | | | | | |
| 96-T | | X | | | X | X | | | | | X | | | | | | | | | |
| 97-P | | X | | | X | X | | | X | | | | | | | | | | | |
| 98-V | | | | X | | | X | X | | | | | | | | | | | | |
| 99-R | | | | | | | | | | | | X | X | | | | | | | |
| 100-F | X | | | | | | | | | | | | | | | X | | | | |
| 101-I | | | | X | | | | X | | X | | | | | | | | | | |
| 102-P | | X | | | X | X | | | X | | | | | | | | | | | |
| 103-L | | | | X | | | X | | | X | | | | | | | | | | |
| 104-S | | X | | | X | | | | X | | X | | | | | | | | | |
| 105-T | | X | | | X | X | | | | | X | | | | | | | | | |
| 106-N | | | | | | | | | | | | | | X | X | | | X | | |
| 107-I | | | | X | | | | X | | X | | | | | | | | | | |
| 108-F | X | | | | | | | | | | | | | | | X | | | | |
| 109-E | | | | | | | | | | | | | | X | | | | X | X | |
| 110-D | | | | | | | | | | | | | | X | X | | | | X | |
| 111-Q | | | | | | | | | | | | | | | X | | | X | X | |
| 112-L | | | | X | | | X | | | X | | | | | | | | | | |
| 113-L | | | | X | | | X | | | X | | | | | | | | | | |
| 114-N | | | | | | | | | | | | | | X | X | | | X | | |
| 115-I | | | | X | | | | X | | X | | | | | | | | | | |
| 116-Q | | | | | | | | | | | | | | | X | | | X | X | |
| 117-F | X | | | | | | | | | | | | | | | X | | | | |
| 118-N | | | | | | | | | | | | | | X | X | | | X | | |
| 119-I | | | | X | | | | X | | X | | | | | | | | | | |
| 120-P | | X | | | X | X | | | X | | | | | | | | | | | |
| 121-T | | X | | | X | X | | | | | X | | | | | | | | | |
| 122-P | | X | | | X | X | | | X | | | | | | | | | | | |
| 123-K | | | | | | | | | | | | | X | | | | X | | | |
| 124-L | | | | X | | | X | | | X | | | | | | | | | | |
| 125-C | | | | | | | | | | | | | | | | | | | | |
| 126-V | | | | X | | | X | X | | | | | | | | | | | | |
| 127-S | | X | | | X | | | | X | | X | | | | | | | | | |
| 128-Y | | | X | | | | | | | | | | | | | X | | | | |
| 129-T | | X | | | X | X | | | | | X | | | | | | | | | |
| 130-I | | | | X | | | | X | | X | | | | | | | | | | |
| 131-W | X | | X | | | | | | | | | | | | | | | | | |
| 132-K | | | | | | | | | | | | | X | | | | X | | | |
| 133-V | | | | X | | | X | X | | | | | | | | | | | | |
| 134-G | | | | | X | X | | | X | | X | | | | | | | | | |
| 135-N | | | | | | | | | | | | | | X | X | | | X | | |
| 136-I | | | | X | | | | X | | X | | | | | | | | | | |
| 137-N | | | | | | | | | | | | | | X | X | | | X | | |
| 138-A | | X | | | | X | | | X | | X | | | | | | | | | |
| 139-P | | X | | | X | X | | | X | | | | | | | | | | | |
| 140-L | | | | X | | | X | | | X | | | | | | | | | | |
| 141-R | | | | | | | | | | | | X | X | | | | | | | |
| 142-T | | X | | | X | X | | | | | X | | | | | | | | | |
| 143-M | | | | | | | X | X | | X | | | | | | | | | | |
| 144-L | | | | X | | | X | | | X | | | | | | | | | | |
| 145-L | | | | X | | | X | | | X | | | | | | | | | | |
| 146-E | | | | | | | | | | | | | | X | | | | X | X | |
| 147-T | | X | | | X | X | | | | | X | | | | | | | | | |
| 148-G | | | | | X | X | | | X | | X | | | | | | | | | |
| 149-G | | | | | X | X | | | X | | X | | | | | | | | | |
| 150-T | | X | | | X | X | | | | | X | | | | | | | | | |
| 151-I | | | | X | | | | X | | X | | | | | | | | | | |
| 152-G | | | | | X | X | | | X | | X | | | | | | | | | |
| 153-Q | | | | | | | | | | | | | | | X | | | X | X | |
| 154-A | | X | | | | X | | | X | | X | | | | | | | | | |
| 155-D | | | | | | | | | | | | | | X | X | | | | X | |
| 156-S | | X | | | X | | | | X | | X | | | | | | | | | |
| 157-S | | X | | | X | | | | X | | X | | | | | | | | | |
| 158-Y | | | X | | | | | | | | | | | | | X | | | | |
| 159-F | X | | | | | | | | | | | | | | | X | | | | |
| 160-K | | | | | | | | | | | | | X | | | | X | | | |
| 161-I | | | | X | | | | X | | X | | | | | | | | | | |
| 162-V | | | | X | | | X | X | | | | | | | | | | | | |
| 163-K | | | | | | | | | | | | | X | | | | X | | | |
| 164-S | | X | | | X | | | | X | | X | | | | | | | | | |
| 165-S | | X | | | X | | | | X | | X | | | | | | | | | |
| 166-N | | | | | | | | | | | | | | X | X | | | X | | |
| 167-F | X | | | | | | | | | | | | | | | X | | | | |
| 168-G | | | | | X | X | | | X | | X | | | | | | | | | |
| 169-Y | | | X | | | | | | | | | | | | | X | | | | |
| 170-N | | | | | | | | | | | | | | X | X | | | X | | |
| 171-L | | | | X | | | X | | | X | | | | | | | | | | |
| 172-L | | | | X | | | X | | | X | | | | | | | | | | |
| 173-Y | | | X | | | | | | | | | | | | | X | | | | |
| 174-C | | | | | | | | | | | | | | | | | | | | |
| 175-P | | X | | | X | X | | | X | | | | | | | | | | | |
| 176-I | | | | X | | | | X | | X | | | | | | | | | | |
| 177-T | | X | | | X | X | | | | | X | | | | | | | | | |
| 178-R | | | | | | | | | | | | X | X | | | | | | | |
| 179-H | | | | | | | | | | | | | X | | | | X | | | |
| 180-F | X | | | | | | | | | | | | | | | X | | | | |
| 181-L | | | | X | | | X | | | X | | | | | | | | | | |
| 182-C | | | | | | | | | | | | | | | | | | | | |
| 183-P | | X | | | X | X | | | X | | | | | | | | | | | |
| 184-F | X | | | | | | | | | | | | | | | X | | | | |
| 185-C | | | | | | | | | | | | | | | | | | | | |
| 186-R | | | | | | | | | | | | X | X | | | | | | | |
| 187-D | | | | | | | | | | | | | | X | X | | | | X | |
| 188-D | | | | | | | | | | | | | | X | X | | | | X | |
| 189-N | | | | | | | | | | | | | | X | X | | | X | | |
| 190-F | X | | | | | | | | | | | | | | | X | | | | |
| 191-C | | | | | | | | | | | | | | | | | | | | |
| 192-A | | X | | | | X | | | X | | X | | | | | | | | | |
| 193-K | | | | | | | | | | | | | X | | | | X | | | |
| 194-V | | | | X | | | X | X | | | | | | | | | | | | |
| 195-G | | | | | X | X | | | X | | X | | | | | | | | | |
| 196-V | | | | X | | | X | X | | | | | | | | | | | | |
| 197-V | | | | X | | | X | X | | | | | | | | | | | | |
| 198-I | | | | X | | | | X | | X | | | | | | | | | | |
| 199-Q | | | | | | | | | | | | | | | X | | | X | X | |
| 200-N | | | | | | | | | | | | | | X | X | | | X | | |
| 201-G | | | | | X | X | | | X | | X | | | | | | | | | |
| 202-K | | | | | | | | | | | | | X | | | | X | | | |
| 203-R | | | | | | | | | | | | X | X | | | | | | | |
| 204-R | | | | | | | | | | | | X | X | | | | | | | |
| 205-L | | | | X | | | X | | | X | | | | | | | | | | |
| 206-A | | X | | | | X | | | X | | X | | | | | | | | | |
| 207-L | | | | X | | | X | | | X | | | | | | | | | | |
| 208-V | | | | X | | | X | X | | | | | | | | | | | | |
| 209-N | | | | | | | | | | | | | | X | X | | | X | | |
| 210-E | | | | | | | | | | | | | | X | | | | X | X | |
| 211-N | | | | | | | | | | | | | | X | X | | | X | | |
| 212-P | | X | | | X | X | | | X | | | | | | | | | | | |
| 213-L | | | | X | | | X | | | X | | | | | | | | | | |
| 214-D | | | | | | | | | | | | | | X | X | | | | X | |
| 215-V | | | | X | | | X | X | | | | | | | | | | | | |
| 216-L | | | | X | | | X | | | X | | | | | | | | | | |
| 217-F | X | | | | | | | | | | | | | | | X | | | | |
| 218-Q | | | | | | | | | | | | | | | X | | | X | X | |
| 219-E | | | | | | | | | | | | | | X | | | | X | X | |
| 220-V | | | | X | | | X | X | | | | | | | | | | | | |

In one preferred embodiment, one amino acid in SEQ ID NO:1 is replaced with an “equivalent amino acid residue” according to table 2. In another preferred embodiment, more than one amino acid in SEQ ID NO:1 are replaced with an “equivalent amino acid residue” according to table 2, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 or 66 amino acids.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

A “polynucleotide according to the present invention” or a “nucleic acid according to the present invention” is any polynucleotide encoding a “polypeptide according to the present invention”, including any polypeptide cited in the claims of the present patent application or the patent granted on the basis of claims of this patent application.

An “integrated genetic element” is a segment of DNA that has been incorporated into a chromosome of a host cell after that element is introduced into the cell through human manipulation. Within the present invention, integrated genetic elements are most commonly derived from linearized plasmids that are introduced into the cells by electroporation or other techniques. Integrated genetic elements are passed from the original host cell to its progeny.

A “cloning vector” is a nucleic acid molecule, such as a plasmid, cosmid, or bacteriophage that has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector.

“Integrative transformants” are recombinant host cells, in which heterologous DNA has become integrated into the genomic DNA of the cells.

A “fusion protein” is a hybrid protein expressed by a nucleic acid molecule comprising nucleotide sequences of at least two genes. For example, a fusion protein can comprise at least part of a polypeptide according to the present invention fused with a polypeptide that binds an affinity matrix. Such a fusion protein provides a means to isolate large quantities of a polypeptide according to the present invention using affinity chromatography.

The term “secretory signal sequence” denotes a DNA sequence that encodes a peptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “expression” refers to the biosynthesis of a gene product. For example, in the case of a structural gene, expression involves transcription of the structural gene into mRNA and the translation of mRNA into one or more polypeptides.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a polypeptide encoded by a splice variant of an mRNA transcribed from a gene.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of less than 10⁹ M⁻¹.

The term “hybridization under stringent conditions” is defined according to Sambrook et al., Molecular Cloning, A Laboratory Manual, Cold Spring Harbor, Laboratory Press (1989), 1.101-1.104. Preferably, hybridization under stringent conditions means that after washing for 1 h with 1 times SSC and 0.1% SDS at 50 degree C., preferably at 55 degree C., more preferably at 62 degree C. and most preferably at 68 degree C., particularly for 1 h in 0.2 times SSC and 0.1% SDS at 50 degree C., preferably at 55 degree C., more preferably at 62 degree C. and most preferably at 68 degree C., a positive hybridization signal is observed. A nucleotide sequence which hybridizes under the above washing conditions with the nucleotide sequence of SEQ ID NO:1 or a nucleotide sequence corresponding thereto in the scope of the degeneracy of the genetic code is encompassed by the present invention.

An “anti-idiotype antibody” is an antibody that binds with the variable region domain of an immunoglobulin. In the present context, an anti-idiotype antibody binds with the variable region of an anti-antibody, and thus, an anti-idiotype antibody mimics an epitope of a polypeptide according to the present invention.

An “antibody fragment” is a portion of an antibody such as F(ab′)₂, F(ab)₂, Fab′, Fab, and the like. Regardless of structure, an antibody fragment binds with the same antigen that is recognized by the intact antibody. For example, an anti-(polypeptide according to the present invention) antibody fragment binds an epitope of a polypeptide according to the present invention. In one embodiment such an antibody is polyclonal, however, in a particular embodiment the antibody is monoclonal.

The term “antibody fragment” also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

A “chimeric antibody” is a recombinant protein that contains the variable domains and complementary determining regions derived from a rodent antibody, while the remainder of the antibody molecule is derived from a human antibody.

“Humanized antibodies” are recombinant proteins in which murine complementarity determining regions of a monoclonal antibody have been transferred from heavy and light variable chains of the murine immunoglobulin into a human variable domain.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). DNAs encoding affinity tags are available from commercial suppliers (e.g., Pharmacia Biotech, Piscataway, N.J.).

A “naked antibody” is an entire antibody, as opposed to an antibody fragment, which is not conjugated with a therapeutic agent. Naked antibodies include both polyclonal and monoclonal antibodies, as well as certain recombinant antibodies, such as chimeric and humanized antibodies.

As used herein, the term “antibody component” includes both an entire antibody and an antibody fragment.

A “target polypeptide” or a “target peptide” is an amino acid sequence that comprises at least one epitope, and that is expressed on a target cell, such as a tumor cell, or a cell that carries an infectious agent antigen. T cells recognize peptide epitopes presented by a major histocompatibility complex molecule to a target polypeptide or target peptide and typically lyse the target cell or recruit other immune cells to the site of the target cell, thereby killing the target cell.

An “antigenic peptide” is a peptide, which will bind a major histocompatibility complex molecule to form an MHC-peptide complex which is recognized by a T cell, thereby inducing a cytotoxic lymphocyte response upon presentation to the T cell. Thus, antigenic peptides are capable of binding to an appropriate major histocompatibility complex molecule and inducing a cytotoxic T cells response, such as cell lysis or specific cytokine release against the target cell which binds or expresses the antigen. The antigenic peptide can be bound in the context of a class I or class II major histocompatibility complex molecule, on an antigen presenting cell or on a target cell.

The term “variant gene” refers to nucleic acid molecules that encode a polypeptide having an amino acid sequence that is a modification of a polypeptide according to the present invention. Such variants include naturally-occurring polymorphisms of genes according to the present invention, as well as synthetic genes that contain conservative amino acid substitutions of the amino acid sequence of a polypeptide according to the present invention. Additional variant forms of genes are nucleic acid molecules that contain insertions or deletions of the nucleotide sequences described herein. A variant gene according to the present invention can be identified by determining whether the gene hybridizes with a nucleic acid molecule having the nucleotide sequence of a polypeptide according to the present invention, or its complement, under stringent conditions.

Alternatively, variant genes can be identified by sequence comparison. Two amino acid sequences have “100% amino acid sequence identity” if the amino acid residues of the two amino acid sequences are the same when aligned for maximal correspondence. Similarly, two nucleotide sequences have “100% nucleotide sequence identity” if the nucleotide residues of the two nucleotide sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art (see, for example, Peruski and Peruski, The Internet and the New Biology: Tools for Genomic and Molecular Research (ASM Press, Inc. 1997), Wu et al. (eds.), “Information Superhighway and Computer Databases of Nucleic Acids and Proteins,” in Methods in Gene Biotechnology, pages 123 151 (CRC Press, Inc. 1997), and Bishop (ed.), Guide to Human Genome Computing, 2nd Edition (Academic Press, Inc. 1998)). Particular methods for determining sequence identity are described below.

Regardless of the particular method used to identify a variant gene, a variant gene encodes a polypeptide which can be characterized by its ability to bind specifically to an anti-(polypeptide according to the invention) antibody.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, alpha-globin, beta-globin, and myoglobin are paralogs of each other.

Due to the imprecision of standard analytical methods, molecular weights and lengths of polymers are understood to be approximate values. When such a value is expressed as “about” X or “approximately” X, the stated value of X will be understood to be accurate to +/−20%, such as +/−10%, for example +/−5%.

### BRIEF DESCRIPTION OF THE DRAWINGS

FIG. 1: illustrates the blood coagulation cascade with all the key factors participating in producing a fibrin-containing blood clot. The cascade is initiated by contact activation (Intrinsic') or tissue factor stimulation (extrinsic'), thus converting zymogens to active proteases. The green arrows indicate activation by thrombin, fXa and fVIIa.

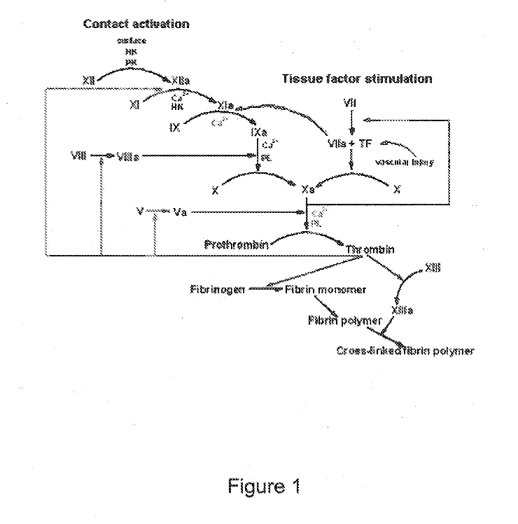

FIG. 2: is a schematic illustration of the purification of the polypeptide of SEQ ID NO:1 from potatoes.

FIG. 3: The coagulation of porcine citrate inhibited plasma was initiated by adding 8 mM CaCl2 in the presence of varying amounts of PIfXa. Coagulation was monitored by turbidity at 590 nm in a spectofotometer.

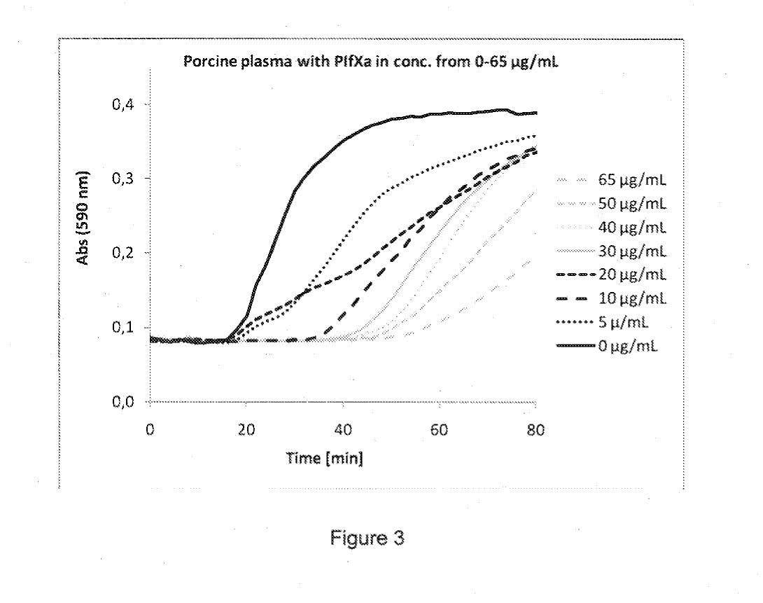

FIG. 4: The coagulation of human citrate inhibited plasma was initiated by adding 8 mM CaCl2 in the presence of varying amounts of PIfXa. Coagulation was monitored by turbidity at 590 nm in a spectofotometer.

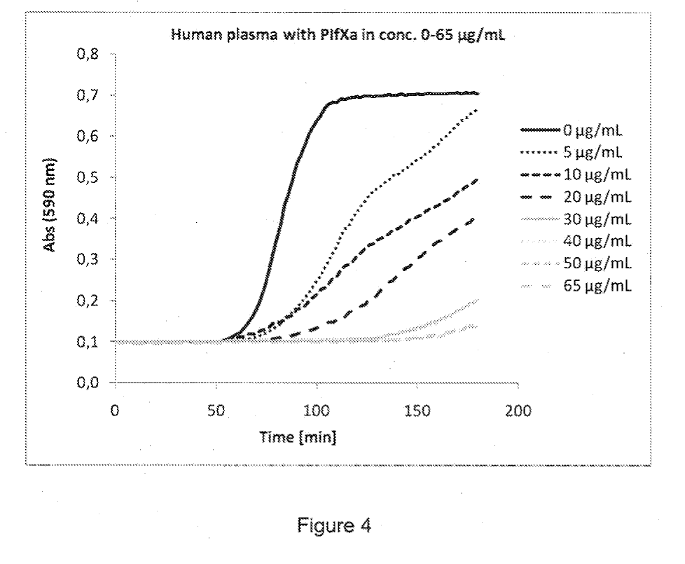

FIG. 5: To verify the biological variation between different donors coagulation of human citrate inhibited plasma from donor 1 was initiated by adding 8 mM CaCl₂ in the absence or presence of 10 and 40 μg/mL PIfXa. Coagulation was monitored by turbidity at 590 nm in a spectofotometer.

FIG. 6: To verify the biological variation between different donors, coagulation of human citrate inhibited plasma from donor 2 was initiated by adding 8 mM CaCl2 in the absence or presence of 10 and 40 μg/mL PIfXa. Coagulation was monitored by turbidity at 590 nm in a spectofotometer.

FIG. 7: To verify the biological variation between different donors coagulation of human citrate inhibited plasma from donor 3 was initiated by adding 8 mM CaCl2 in the absence or presence of 10 and 40 μg/mL PIfXa. Coagulation was monitored by turbidity at 590 nm in a spectofotometer.

FIG. 8: To verify the biological variation between different donors coagulation of human citrate inhibited plasma from donor 4 was initiated by adding 8 mM CaCl2 in the absence or presence of 10 and 40 μg/mL PIfXa. Coagulation was monitored by turbidity at 590 nm in a spectofotometer.

FIG. 9: Amidolytic activity assay for fXa in the absence and presence of PIfXa. 0.25 IU/mL heparin was used as a negative control for inhibition. Furthermore a no-enzyme control was added.

FIG. 10: Amidolytic activity assay for thrombin in the absent and presence of PIfXa. 0.25 IU/mL heparin was used as a negative control for inhibition. Furthermore a no-enzyme control was added.

FIG. 11: Amidolytic activity assay for fVIIa in the absent and presence of PIfXa. 0.25 IU/mL heparin was used as a negative control for inhibition. Furthermore a no-enzyme control was added.

FIG. 12: Different amounts of antibody added to the PIfXa, fXa solution 5 min into the amidolytic activity assay to demonstrate that the inhibitory effect could be reversed at least.

FIG. 13: Coagulation time of citrated human plasma incubated with LMWH, fondaparinux and PIfXa calculated by the aPTT assay. The coagulation was initiated by the addition of 7 mM CaCl2.

FIG. 14: Dose-response curve of PIfXa converted to IU/mL LMWH from a standard curve.

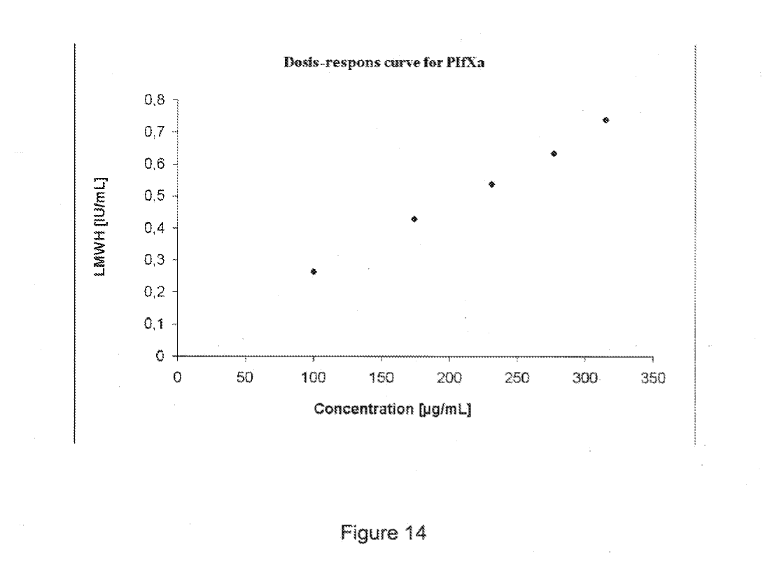

FIG. 15: fX, Russell's Viper Venom (RVV) and PIfXa on a 12% SDS PAGE gel. + and − indicates reducing and non-reducing conditions, respectively.

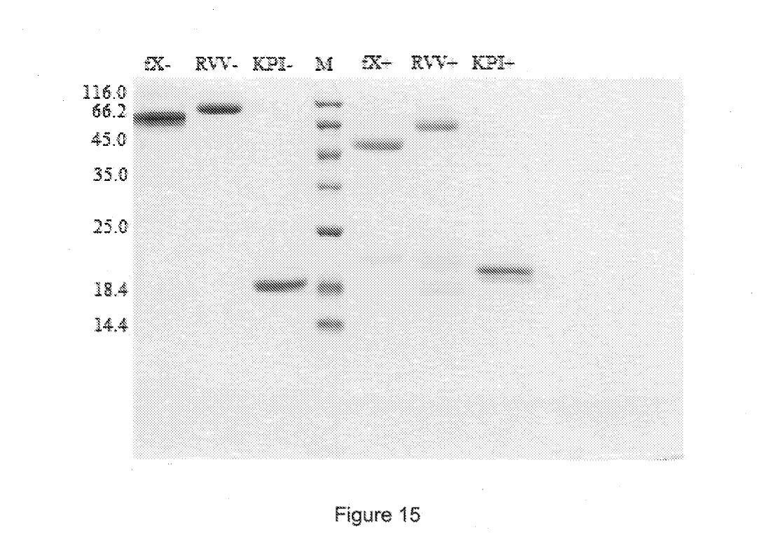

FIG. 16: Different mixtures of fX, Russell's Viper Venom (RVV) and PIfXa on a 12% SDS PAGE gel. + and − indicates reducing and non-reducing conditions, respectively. fX and PIfXa was incubated for 10 min at room temperature before RVV was added.

FIG. 17: Bleeding time within 30 min of tail clipping from the pilot rat experiment. Each group contained n=3 animals, except the ctrl and hep groups where n=4.

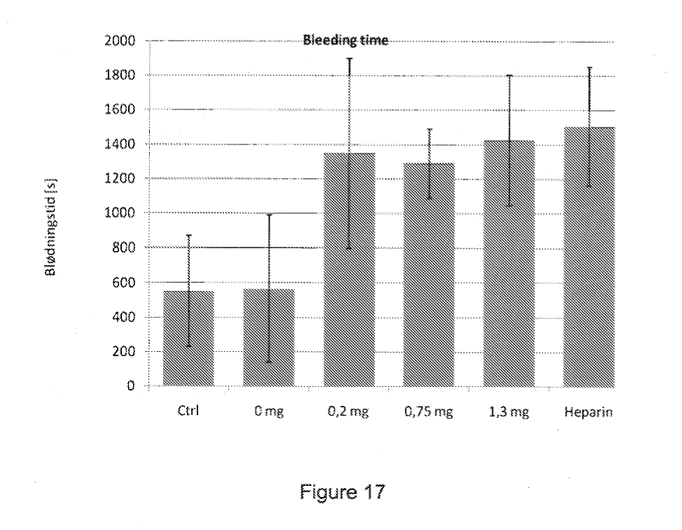

FIG. 18: Haemoglobin concentrations of collected blood from the pilot experiment measured by absorption at 410 nm. Each group contained n=3 animals, except the ctrl and hep groups where n=4.

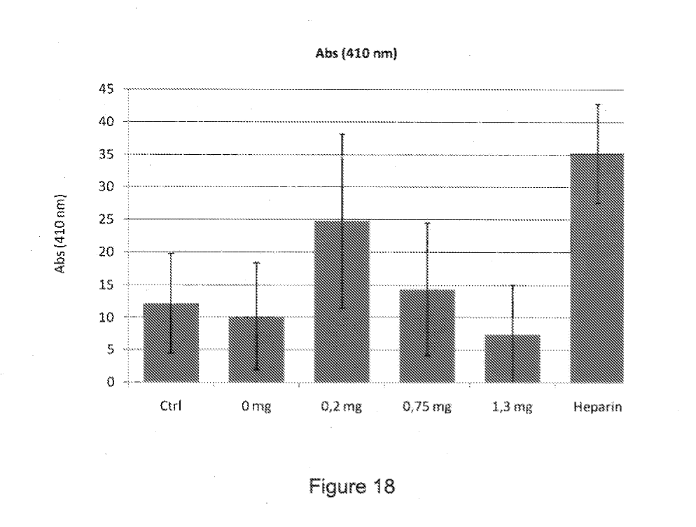

FIG. 19: Bleeding time within 30 min of tail clipping from the animal experiment. The data from the pilot experiment is included. The concentrations have been divided into three groups. The low dose contains 0.063 mg and 0.125 mg (n=14). The medium dose contains 0.2 and 0.25 mg (n=10) and the high dose contains 0.5, 0.75 and 1.3 mg (n=13). Heparin contained n=10 animals and the control, 0 mg group contained 21 animals.

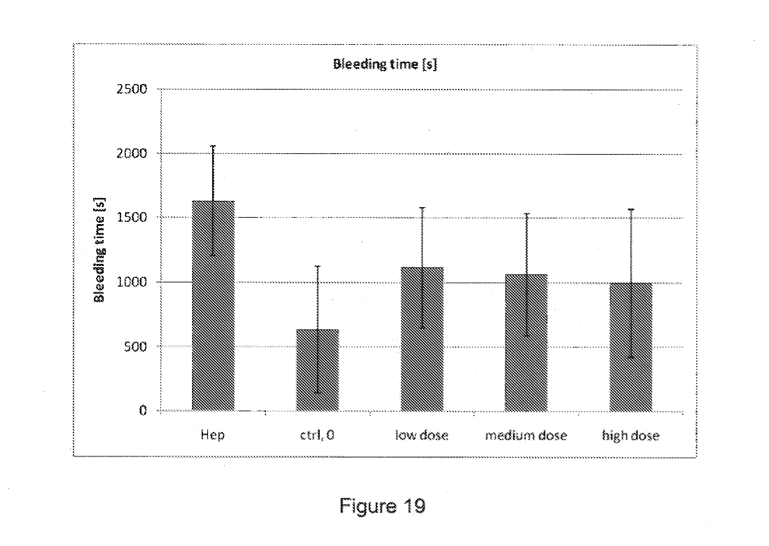

FIG. 20: Haemoglobin concentrations of collected blood. The concentrations have been divided into three groups. The low dose contains 0.063 mg and 0.125 mg (n=14). The medium dose contains 0.2 and 0.25 mg (n=10) and the high dose contains 0.5, 0.75 and 1.3 mg (n=13). Heparin contained n=10 animals and the control, 0 mg group contained 21 animals.

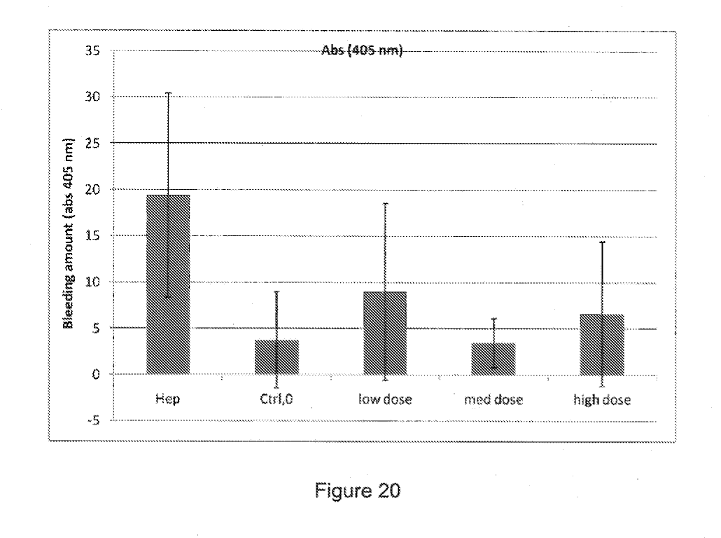

FIG. 21: 12% SDS PAGE gel after extraction of proteins from Nicotiana benthamiana leafs. Lane 1: protein marker, lane 2: extracted proteins from half of a leaf infiltrated with pCAMBIA2300. Lane 3 and 4: Extracted proteins from half of a leaf infiltrated with pCAMBIA2300-GFP. Lane 5: Extracted proteins from half of a leaf infiltrated with pCAMBIA2300-KPI A-k1. KPI A-k1 is PIfXa.

### DETAILED DESCRIPTION OF THE INVENTION

### Polypeptide Sequence of SEQ ID NO:1

The present invention in one aspect relates to a nucleotide sequence encoding a polypeptide according to the present invention as disclosed herein. In one embodiment, the polypeptide has Kunitz-type serine protease inhibitor activity. Kunitz-type protease inhibitors consist of two families: The bovine pancreatic trypsin inhibitor (BPTI) family comprising TFPI (tissue factor pathway inhibitor), and the soybean trypsin inhibitor (STI). STIs are found in numerous plants and have a molecular mass of 20-22 kDa. The best conserved region is found near the N-terminal and is used as a signature pattern consisting of [LIVM]-x-D-{EK}-[EDHNTY]-[DG]-[RKHDENQ]-x-[LIVM]-x-{E}-x-x-x-Y-x-[LIVM]. In plants, the functions of Kunitz-type protease inhibitors include enzyme inhibition, protein storage and pathogen or insect defense.

In one embodiment of the invention, the polypeptide with Kunitz-type serine protease inhibitor activity described herein belongs to the BPTI-family of kunitz-type proteases. In another embodiment of the present invention, the polypeptide with Kunitz-type serine protease inhibitor activity described herein belongs to the STI-family of kunitz-type proteases.

The polypeptide according to the present invention was originally purified from potato tuber (Solanum tuberosum). About 40% of the total protein content in the potato tuber consists of protease inhibitors of which a very large proportion is Kunitz-type. This makes the potato (Solanum tuberosum) an easily accessible source for this type of inhibitor.

The polypeptide according to the invention can be linked to a carrier, such as a solid support or semi-solid support. The polypeptide can be covalently or non-covalently linked to any such carrier, for example a surface of a material desirably displaying the polypeptides according to the invention.

The invention further relates to a polypeptide according to the present invention fused to an affinity tag. Examples of such affinity tags are known from the literature and can be selected from the group comprising for example: His-tag, protein A tag, Avidin/streptavidin, Avidin/steptavidin optionally biotinylated, protein G, GluthationeS-tranferase, dihyfrofolate reductase (DHFR), Green fluorescent protein (GFP), polyarginine, polycysteine, c-myc, calmodulin binding protein, influenzavirus hemagglutinin.

The invention also encompasses polypeptides, wherein one or more amino acid residues are modified, wherein said one or more modification(s) are preferably selected from the group consisting of in vivo or in vitro chemical derivatization, such as acetylation or carboxylation, glycosylation, such as glycosylation resulting from exposing the polypeptide to enzymes which affect glycosylation, for example mammalian glycosylating or deglycosylating enzymes, phosphorylation, such as modification of amino acid residues which results in phosphorylated amino acid residues, for example phosphotyrosine, phosphoserine and phosphothreonine.

The polypeptide according to the invention can comprise one or more amino acids independently selected from the group consisting of naturally occurring L-amino acids, naturally occurring D-amino acids as well as non-naturally occurring, synthetic amino acids.

One or more amino acid residues of the polypeptide of the present invention are modified so as to preferably improve the resistance to proteolytic degradation and stability or to optimize solubility properties or to render the polypeptide more suitable as a therapeutic agent.

The invention also relates to polypeptides of the invention where blocking groups are introduced in order to protect and/or stabilize the N- and/or C-termini of the polypeptide from undesirable degradation. Such blocking groups may be selected from the group comprising branched or non-branched alkyl groups and acyl groups, such as formyl and acetyl groups, as well substituted forms thereof, such as acetamidomethyl.

The invention also relates to the following:

The polypeptide according to present invention, wherein the one or more blocking groups are selected from N-terminal blocking groups comprising desamino analogs of amino acids, which are either coupled to the N-terminus of the peptide or used in place of the N-terminal amino acid residue.

The polypeptide according to present invention, wherein the one or more blocking groups are selected from C-terminal blocking groups wherein the carboxyl group of the C-terminus is either incorporated or not, such as esters, ketones, and amides, as well as descarboxylated amino acid analogues.

The polypeptide according to present invention, wherein the one or more blocking groups are selected from C-terminal blocking groups comprising ester or ketone-forming alkyl groups, such as lower (C₁ to C₆) alkyl groups, for example methyl, ethyl and propyl, and amide-forming amino groups, such as primary amines (—NH₂), and mono- and di-alkylamino groups, such as methylamino, ethylamino, dimethylamino, diethylamino, methylethylamino, and the like.

The polypeptide according to present invention, wherein free amino group(s) at the N-terminal end and free carboxyl group(s) at the termini can be removed altogether from the polypeptide to yield desamino and descarboxylated forms thereof without significantly affecting the biological activity of the polypeptide.

The invention further relates to modifications and derivatives of the polypeptide according to the invention, nucleotides encoding said polypeptides, vectors comprising said nucleotides, host cells transformed with said vectors and transgenic organisms comprising said cells.

### Biological Functions of the Polypeptide According to the Present Invention

In one embodiment of the present invention, the polypeptide according to the present invention acts by blocking thrombin generation and clot formation by direct and specific Factor Xa (fXa) inhibition. Thereby the thrombin-mediated activation of platelets and other coagulation factors is diminished without interfering with the basal level of thrombin activity necessary for normal hemostasis. Thus, to control the blood clotting cascade specifically, fXa is a potential target candidate due to its activating effects on the key molecule thrombin.

A number of parenteral and oral fXa inhibitors have been described and is currently in various stages of development. fXa inhibitors can be divided into 1) Natural source proteins, including tick anticoagulant peptide (TAP), 2) Synthetic peptides and low molecular weight organic molecules including DX9065a, and 3) Synthetic heparinomimetics that are indirect selective fXa inhibitors. In one embodiment, the polypeptide according to the present invention is a natural source protein, for example extracted from potato tuber (Solanum tuberosum). In another embodiment, the polypeptide according to the present invention is a synthetic peptide.

In one embodiment, the polypeptide according to the present invention is a kunitz-type serine protease inhibitor with specific activity against fXa, thus inhibiting the blood clotting cascade. Thus, by inhibiting this fXa protease, the polypeptide according to the present invention can modulate blood fluidity to control hemostasis.

Patient Groups and Clinical Indications Benefiting from Treatment with the Polypeptide According to the Present Invention

Below is disclosed a non-limiting list of the multitude of patient groups that are likely to benefit from treatment with anticoagulant therapy such as in the prophylaxis, cure and amelioration of disease or symptoms. The polypeptide according to the present invention in the form of a pharmaceutical composition will likely be used in anticoagulation therapy for the following medical conditions. The list is non-limiting in that other conditions might prove treatable with the polypeptide according to the present invention.

Acute coronary syndrome, e.g., myocardial infarction. Acute myocardial infarction (MI) is defined as death or necrosis of myocardial cells. It is a diagnosis at the end of the spectrum of myocardial ischemia (restriction of blood supply) or acute coronary syndromes. Myocardial infarction occurs when myocardial ischemia exceeds a critical threshold and overwhelms myocardial cellular repair mechanisms that are designed to maintain normal operating function and hemostasis. Critical myocardial ischemia may occur as a result of increased myocardial metabolic demand and/or decreased delivery of oxygen and nutrients to the myocardium via the coronary circulation. An interruption in the supply of myocardial oxygen and nutrients occurs when a thrombus is superimposed on an ulcerated or unstable atherosclerotic plaque and results in coronary occlusion. Conditions associated with increased myocardial metabolic demand include extremes of physical exertion, severe hypertension (including forms of hypertrophic obstructive cardiomyopathy), and severe aortic valve stenosis. Other cardiac valvular pathologies and low cardiac output states associated with a decreased aortic diastolic pressure, which is the prime component of coronary perfusion pressure, can also precipitate MI. The most common etiology of MI is a thrombus superimposed on a ruptured or unstable atherosclerotic plaque. Myocardial infarction is the leading cause of death in the United States (US) as well as in most industrialized nations throughout the world. In general, MI can occur at any age, but its incidence rises with age. The actual incidence is dependent upon predisposing risk factors for atherosclerosis. Six primary risk factors have been identified with the development of atherosclerotic coronary artery disease and MI: hyperlipidemia, diabetes mellitus, hypertension, smoking, male gender, and family history of atherosclerotic arterial disease. Thus, in patients at high risk for developing MI, administration of direct acting antithrombin drugs such as the specific Xa inhibiting polypeptide described herein could prevent the occlusion of the coronary arteries before and after a MI-attack.

Cerebrovascular accident/stroke. The presence of a blood clot in the brain causes an acute ischemic stroke due to sustained decreased blood flow to parts of the brain. Prior to this event, a TIA (transient cerebral ischemic attack) can occur, which is caused by the temporary disturbance of blood supply to a restricted area of the brain, resulting in brief neurologic dysfunction that usually persists for less than 24 hours. This is called a ‘mini stroke’, and can be a warning of an approaching ischemic stroke. The most frequent symptoms include loss of vision, difficulty speaking (aphasia); weakness on one side of the body (hemiparesis); numbness or tingling (paresthesia), usually on one side of the body; and loss of consciousness. If there are neurological symptoms persisting for more than 24 hours, it is classified as a cerebrovascular accident, or stroke. It is important to rule out a hemorrhagic stroke before initiating anticoagulant therapy.

Atrial fibrillation. Atrial fibrillation is a cardiac arrhythmia (abnormal heart rhythm) that involves the two upper chambers (atria) of the heart. Atrial fibrillation is the most common arrhythmia; risk increases with age, with 8% of people over 80 having AF. In atrial fibrillation, the electrical impulses that are normally generated by the sinoatrial node are replaced by disorganized activity in the atria, leading to irregular conduction of impulses to the ventricles that generate the heartbeat. The result is an irregular heartbeat. This may be continuous (persistent or permanent AF) or alternating between periods of a normal heart rhythm (paroxysmal AF). The predominant cause of strokes in patients with atrial fibrillation is embolization of a clot from the left atrium. Atrial fibrillation is the underlying cause of 30,000 to 40,000 embolic strokes per year in the United States. The incidence of these strokes increases with age, rising from 1.5 percent in patients aged 50 to 59 years to 23.5 percent in patients aged 80 to 89 years. Risk factors for stroke in patients with atrial fibrillation include a history of transient ischemic attack or stroke, age greater than 65 years, a history of hypertension, the presence of a prosthetic heart valve (mechanical or tissue), rheumatic heart disease, left ventricular systolic dysfunction, or diabetes. Most atrial fibrillation-derived strokes occur within the first 72 hours after medical (pharmacologic) or electrical cardioversion, and is presumed to be due to the presence of left atrial thrombi at the time of cardioversion, rather than to the method used. Therefore, the use of antithrombotic therapy in patients with atrial fibrillation is used to prevent thromboembolic complications in patients with atrial fibrillation.

Angina pectoris. Angina pectoris is a Latin phrase and translates as ‘tight chest’. People with angina experience pain in the centre of the chest. In most cases, the cause of angina is coronary atherosclerosis: the thickening of arteries that supply blood, oxygen and nutrients to the heart. Stable angina develops during exertion and resolves at rest. In contrast with stable angina, unstable angina occurs suddenly, often at rest or with minimal exertion, or at lesser degrees of exertion than the individual's previous angina (“crescendo angina”). New onset angina is also considered unstable angina, since it suggests a new problem in a coronary artery. Thus, the administration of blood thinning medicine will prevent formation of coronary occlusion by thrombus formation associated with the atherosclerotic plaque.

Deep-vein thrombosis and pulmonary embolism. Deep vein thrombosis (DVT) refers to the formation of a thrombus (blood clot) within a deep vein, commonly in the thigh or calf. Several factors contribute to formation of clots in veins: 1) Stasis, or stagnant blood flow through veins. This increases the contact time between blood and vein wall irregularities. It also prevents naturally occurring anticoagulants from mixing in the blood. Prolonged bed rest or immobility promotes stasis. 2) Coagulation. Coagulation is encouraged by the presence of tissue debris, collagen or fats in the veins. Orthopaedic surgery often releases these materials into the blood system. Although venous thromboembolic disease can develop after any major surgery, people who have surgery on the lower extremities are especially vulnerable. 3) Damage to the vein walls. This can occur during surgery as the physician retracts soft tissues as part of the procedure. This can also break intercellular bridges and release substances that promote blood clotting. Other factors that may contribute to the formation of thrombi in the veins include age, previous history of DVT or pulmonary embolism, metastatic malignancy, vein disease (such as varicose veins), smoking, estrogen usage or current pregnancy, obesity and genetic factors. Thus, for the events causing increased risk of pathological blood clotting, the administration of antithrombus compounds such as the polypeptide described herein is advisable for pharmacologic prophylaxis of DVT.

Peripheral arterial occlusion. For treating blood clots that are blocking a peripheral artery, e.g. in the leg, anticoagulant therapy is also employed. Often this is caused by atherosclerosis of the vessels, which when occurring in the leg can cause Intermittent Claudication, which is a cramping sensation in the foot or calf from low oxygen supply.

Recurrent arterial thrombosis or embolism. In this case, secondary prophylaxis is highly warranted. Thrombophilia (tendency to develop thrombosis) often expresses itself with recurrent thromboses.

Cardiopulmonary bypass for heart surgery and hemodialysis in kidney failure. Open-heart surgery with cardiopulmonary bypass (CPB) causes transient activation of the coagulation and fibrinolytic systems. Anticoagulant-coated CPB circuits improve the biocompatibility of CPB during heart surgery, as reflected by significantly reduced levels of circulating complement factors and interleukin-6. Thus, biocompatible surfaces mimic critical properties of the vascular endothelium to provide thromboresistance and biocompatibility for extracorporeal circuits. Therefore, the use of the polypeptide described herein might prove useful for these purposes. Further, this is also indicated by having a central vein catheter (CVK).

Patient groups that cannot be treated with conventional antithrombotic therapy. In another embodiment, the polypeptide according to the present invention can be used in patients in whom other agents acting on platelet aggregation, coagulation or fibrinolysis is not tolerated in the patient. An example is aspirin, for which there are many contraindications such as allergy to ibuprofen, asthma and NSAID-precipitated bronchospasm, peptic ulcers, kidney disease, gastritis, hemophilia, hyperthyroidism and glycose-6-phosphate deydrogenase deficiency in which aspirin can cause hemolytic anemia. Further, aspirin have adverse effects such as gastrointestinal complaints (stomach upset, dydpepsia, heartburn, small blood loss, ulceration), central effects (dizziness, tinnitus, hearing loss, vertigo and headaches) and nephritis. Further, hypersensitivity might occur to any drug. Hypersensitivity refers to undesirable (damaging, discomfort-producing and sometimes fatal) reactions produced by the normal immune system.

Angioplasty. Angioplasty is the mechanical widening of a narrowed or totally obstructed blood vessel. These obstructions are often caused by atherosclerosis. Angioplasty has come to include all manner of vascular interventions typically performed in a minimally invasive or percutaneous method. Procedures include coronary angioplasty of the coronary arteries of the heart (percutaneous coronary intervention), peripheral angioplasty often performed in leg vessels (percutaneous transluminal angioplasty), renal artery angioplasty (percutaneous transluminal renal angioplasty) and carotid angioplasty on the artery that supplies the head and neck. During these procedures, simultaneous inhibition of the blood clotting system is desired to minimize the risk of accompanying thrombosis.

Managing coagulation: coagulation factor disorder. The best-known coagulation factor disorders are the hemophilias. The three main forms are hemophilia A (factor VIII deficiency), hemophilia B (factor IX deficiency or “Christmas disease”) and hemophilia C (factor XI deficiency, mild bleeding tendency). Together with von Willebrand disease (which behaves more like a platelet disorder except in severe cases), these conditions predispose to bleeding. Most hemophilias are inherited. In liver failure (acute and chronic forms) there is insufficient production of coagulation factors by the liver; this may increase bleeding risk. Thus, when treating bleeding disorders pharmaceutically, the polypeptide according to the present invention can be used for optimally managing coagulation in individuals at increased risk of bleeding, by reversing the effect of procoagulants. Procoagulant compounds include coagulation factor concentrates, prothrombin complex concentrate, cryoprecipitate and fresh frozen plasma and recombinant activated human factor VII. Tranexamic acid and aminocaproic acid inhibit fibrinolysis, and lead to a de facto reduced bleeding rate. Further, major blood loss also causes an imbalance in the coagulation components and need managing due to the increased consumption of (anti-)coagulation factors.

Managing coagulation: platelet disorder. Platelet conditions may be inborn or acquired. Some inborn platelet pathologies are Glanzmann's thrombasthenia, Bernard-Soulier syndrome (abnormal glycoprotein Ib-IX-V complex), gray platelet syndrome (deficient alpha granules) and delta storage pool deficiency (deficient dense granules). Most are rare conditions. von Willebrand disease is due to deficiency or abnormal function of von Willebrand factor. Most inborn platelet pathologies predispose to hemorrhage. Decreased platelet numbers may be due to various causes, including insufficient production (e.g. in myelodysplastic syndrome or other bone marrow disorders), destruction by the immune system (immune thrombocytopenic purpura/ITP), and consumption due to various causes (thrombotic thrombocytopenic purpura/TTP, hemolytic-uremic syndrome/HUS, paroxysmal nocturnal hemoglobinuria/PNH, disseminated intravascular coagulation/DIC, heparin-induced thrombocytopenia/HIT). Most consumptive conditions lead to platelet activation, and some are associated with thrombosis.

Artificial/prosthetic heart valves. An artificial heart valve is a device which is implanted in the heart of patients who suffer from valvular diseases in their heart. Prosthetic valves can be mechanical or bioprosthetic. Mechanical heart valves are considered to be extremely durable in comparison to their bioprosthetic counterparts. Valves are integral to the normal physiological functioning of the human heart; to induce largely unidirectional flow through them. Natural heart valves may become dysfunctional due to a variety of pathological causes. One of the major drawbacks of mechanical heart valves is that patients with these implants have increased tendency of thrombus formation. Clots formed by red blood cell (RBC) and platelet damage can block up blood vessels and lead to very serious consequences. Clotting occurs in one of three basic pathways: tissue factor exposure, platelet activation, or contact activation by foreign materials. Because all models experience high stresses, patients with mechanical heart valve implants require anti-coagulation therapy. Bioprosthetics are less prone to develop blood clotting, but the trade-off concerning durability generally favors their use in patients older than age 55. Thus, artificial valve patients require consistent anti-coagulation therapy.

Heart valve disease. Various conditions affecting the heart valves can increase the risk of thrombosis. These include infected valves from bacterial endocarditis, rheumatic mitral valve disease, mitral stenosis, mitral valve prolapse, mitral annular calcification and isolated aortic valve disease.

Thrombophilia. Thrombophilia is the propensity to develop thrombosis (blood clots) due to an abnormality in the system of coagulation. The causes of thrombophilia are amongst others: Factor V Leiden is the name given to a variant of human factor V that causes a hypercoagulability disorder. In this disorder the Leiden variant of factor V, cannot be inactivated by activated protein C. Factor V Leiden is the most common hereditary hypercoagulability disorder amongst Eurasians. Factor V Leiden is an autosomal dominant condition in which the coagulation factor cannot be destroyed by aPC. Mutation of the gene encoding factor V—a single nucleotide substitution of adenine for guanine—changes the protein's 506th amino acid from arginine to glutamine. Since this amino acid is normally the cleavage site for aPC, the mutation prevents efficient inactivation of factor V. When factor V remains active, it facilitates overproduction of thrombin leading to excess fibrin generation and excess clotting. The excessive clotting that occurs in this disorder is almost always restricted to the veins, where the clotting may cause a deep vein thrombosis (DVT). If the venous clots break off, these clots can travel through the heart to the lung, where they block a pulmonary blood vessel and cause a pulmonary embolism. Women with the disorder have an increased risk of miscarriage and stillbirth. This disorder does not increase the formation of clots in arteries that can lead to stroke or heart attack, though a transient ischemic attack may occur. Prothrombin mutation: The prothrombin gene mutation is called Factor II mutation. Factor II mutation is congenital. The gene may be inherited heterozygous, or much more rarely, homozygous, and is not related to gender or blood type. Homozygous mutations increase the risk of thrombosis more than heterozygous mutations, but the relative increased risk is not well documented. High homocysteine levels due to MTHFR mutation or vitamin deficiency (vitamins B6, B12 and folic acid). Antiphospholipid syndrome (or antiphospholipid antibody syndrome) (APS) is a disorder of coagulation, which causes thrombosis in both arteries and veins, as well as pregnancy-related complications such as miscarriage, preterm delivery, or severe preeclampsia. The syndrome occurs due to the autoimmune production of antiphospholipid antibodies (aPL). In particular the disease is characterized by anti-cardiolipin and anti-132 glycoprotein I antibodies. In APS patients, the most common venous event is deep vein thrombosis of the lower extremities (blood clot of the deep veins of the legs) and the most common arterial event is stroke. Renal disease renal loss of antithrombin. More rare causes of thrombophilia include plasminogen and fibrinolysis disorders, paroxysmal nocturnal hemoglobinuria, protein C deficiency, protein S deficiency, and antithrombin III deficiency.

Extrinsic factors affecting thrombosis: Many cases of thrombosis are due to acquired extrinsic problems (surgery, cancer, chemotherapy, immobility, obesity, economy class syndrome). “Economy class syndrome” during air travel is a combination of immobility and relative dehydration.

Thus, the polypeptide according to the present invention is aimed at providing a method for therapy of any of the above-listed conditions, and possibly further unlisted examples apply.

### In Vivo Administration and Dosage of the Polypeptide According to the Present Invention

Any suitable route of administration may be employed for providing a mammal, especially a human, with an effective dose of a compound of the present invention. For example, oral, rectal, vaginal, topical, parenteral, ocular, pulmonary, nasal, and the like may be employed. Other examples of administration include sublingually, intravenously, intramuscularly, intrathecally, subcutaneously, cutaneously and transdermally administration. In one preferred embodiment the administration comprises inhalation, injection or implantation. The administration of the compound according to the present invention can result in a local (topical) effect or a bodywide (systemic) effect.

Dosage forms include tablets, troches, dispersions, suspensions, solutions, capsules, creams, ointments, aerosols, and the like. Preferably compounds of the invention are administered orally or intravenously.

The effective dosage of active ingredient employed may vary depending on the particular compound employed, the mode of administration, the condition being treated and the severity of the condition being treated. Such dosage may be ascertained readily by a person skilled in the art.

In one embodiment the compounds of the present invention is administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligram to about 50 milligrams. In the case of a 70 kg adult human, the total daily dose will generally be from about 1 milligram to about 350 milligrams. For a particularly potent compound, the dosage for an adult human may be as low as 0.1 mg. The dosage regimen may be adjusted within this range or even outside of this range to provide the optimal therapeutic response.

Oral administration will usually be carried out using tablets. Examples of doses in tablets are 0.5 mg, 1 mg, 2 mg, 5 mg, 10 mg, 25 mg, 50 mg, 100 mg, and 250 mg. Other oral forms can also have the same dosages (e.g. capsules).

The compound according to the present invention is given in an effective amount to an individual in need there of. The amount of compound according to the present invention in one preferred embodiment is in the range of from about 0.01 milligram per kg body weight per dose to about 20 milligram per kg body weight per dose, such as from about 0.02 milligram per kg body weight per dose to about 18 milligram per kg body weight per dose, for example from about 0.04 milligram per kg body weight per dose to about 16 milligram per kg body weight per dose, such as from about 0.06 milligram per kg body weight per dose to about 14 milligram per kg body weight per dose, for example from about 0.08 milligram per kg body weight per dose to about 12 milligram per kg body weight per dose, such as from about 0.1 milligram per kg body weight per dose to about 10 milligram per kg body weight per dose, such as from about 0.2 milligram per kg body weight per dose to about 10 milligram per kg body weight per dose, for example from about 0.3 milligram per kg body weight per dose to about 10 milligram per kg body weight per dose, such as from about 0.4 milligram per kg body weight per dose to about 10 milligram per kg body weight per dose, for example from about 0.5 milligram per kg body weight per dose to about 10 milligram per kg body weight per dose, such as from about 0.6 milligram per kg body weight per dose to about 10 milligram per kg body weight per dose, for example from about 0.7 milligram per kg body weight per dose to about 10 milligram per kg body weight per dose, such as from about 0.8 milligram per kg body weight per dose to about 10 milligram per kg body weight per dose, for example from about 0.9 milligram per kg body weight per dose to about 10 milligram per kg body weight per dose, such as from about 1.0 milligram per kg body weight per dose to about 10 milligram per kg body weight per dose, for example from about 1.2 milligram per kg body weight per dose to about 10 milligram per kg body weight per dose, such as from about 1.4 milligram per kg body weight per dose to about 10 milligram per kg body weight per dose, for example from about 1.6 milligram per kg body weight per dose to about 10 milligram per kg body weight per dose, such as from about 1.8 milligram per kg body weight per dose to about 10 milligram per kg body weight per dose, for example from about 2.0 milligram per kg body weight per dose to about 10 milligram per kg body weight per dose, such as from about 2.2 milligram per kg body weight per dose to about 10 milligram per kg body weight per dose, for example from about 2.4 milligram per kg body weight per dose to about 10 milligram per kg body weight per dose, such as from about 2.6 milligram per kg body weight per dose to about 10 milligram per kg body weight per dose, for example from about 2.8 milligram per kg body weight per dose to about 10 milligram per kg body weight per dose, such as from about 3.0 milligram per kg body weight per dose to about 10 milligram per kg body weight per dose, for example from about 3.2 milligram per kg body weight per dose to about 10 milligram per kg body weight per dose, such as from about 3.4 milligram per kg body weight per dose to about 10 milligram per kg body weight per dose, for example from about 3.6 milligram per kg body weight per dose to about 10 milligram per kg body weight per dose, such as from about 3.8 milligram per kg body weight per dose to about 10 milligram per kg body weight per dose, for example from about 4.0 milligram per kg body weight per dose to about 10 milligram per kg body weight per dose, such as from about 4.2 milligram per kg body weight per dose to about 10 milligram per kg body weight per dose, for example from about 4.4 milligram per kg body weight per dose to about 10 milligram per kg body weight per dose, such as from about 4.6 milligram per kg body weight per dose to about 10 milligram per kg body weight per dose, for example from about 4.8 milligram per kg body weight per dose to about 10 milligram per kg body weight per dose, such as from about 5.0 milligram per kg body weight per dose to about 10 milligram per kg body weight per dose, for example from about 5.2 milligram per kg body weight per dose to about 10 milligram per kg body weight per dose, such as from about 5.4 milligram per kg body weight per dose to about 10 milligram per kg body weight per dose, for example from about 5.6 milligram per kg body weight per dose to about 10 milligram per kg body weight per dose, such as from about 5.8 milligram per kg body weight per dose to about 10 milligram per kg body weight per dose, for example from about 6.0 milligram per kg body weight per dose to about 10 milligram per kg body weight per dose, such as from about 6.2 milligram per kg body weight per dose to about 10 milligram per kg body weight per dose, for example from about 6.4 milligram per kg body weight per dose to about 10 milligram per kg body weight per dose, such as from about 6.6 milligram per kg body weight per dose to about 10 milligram per kg body weight per dose, for example from about 6.8 milligram per kg body weight per dose to about 10 milligram per kg body weight per dose, such as from about 7.0 milligram per kg body weight per dose to about 10 milligram per kg body weight per dose, for example from about 7.2 milligram per kg body weight per dose to about 10 milligram per kg body weight per dose, such as from about 7.4 milligram per kg body weight per dose to about 10 milligram per kg body weight per dose, for example from about 7.6 milligram per kg body weight per dose to about 10 milligram per kg body weight per dose, such as from about 7.8 milligram per kg body weight per dose to about 10 milligram per kg body weight per dose, for example from about 8.0 milligram per kg body weight per dose to about 10 milligram per kg body weight per dose, such as from about 0.2 milligram per kg body weight per dose to about 8 milligram per kg body weight per dose, for example from about 0.3 milligram per kg body weight per dose to about 8 milligram per kg body weight per dose, such as from about 0.4 milligram per kg body weight per dose to about 8 milligram per kg body weight per dose, for example from about 0.5 milligram per kg body weight per dose to about 8 milligram per kg body weight per dose, such as from about 0.6 milligram per kg body weight per dose to about 8 milligram per kg body weight per dose, for example from about 0.7 milligram per kg body weight per dose to about 8 milligram per kg body weight per dose, such as from about 0.8 milligram per kg body weight per dose to about 8 milligram per kg body weight per dose, for example from about 0.9 milligram per kg body weight per dose to about 8 milligram per kg body weight per dose, such as from about 1.0 milligram per kg body weight per dose to about 8 milligram per kg body weight per dose, for example from about 1.2 milligram per kg body weight per dose to about 8 milligram per kg body weight per dose, such as from about 1.4 milligram per kg body weight per dose to about 8 milligram per kg body weight per dose, for example from about 1.6 milligram per kg body weight per dose to about 8 milligram per kg body weight per dose, such as from about 1.8 milligram per kg body weight per dose to about 8 milligram per kg body weight per dose, for example from about 2.0 milligram per kg body weight per dose to about 8 milligram per kg body weight per dose, such as from about 2.2 milligram per kg body weight per dose to about 8 milligram per kg body weight per dose, for example from about 2.4 milligram per kg body weight per dose to about 8 milligram per kg body weight per dose, such as from about 2.6 milligram per kg body weight per dose to about 8 milligram per kg body weight per dose, for example from about 2.8 milligram per kg body weight per dose to about 8 milligram per kg body weight per dose, such as from about 3.0 milligram per kg body weight per dose to about 8 milligram per kg body weight per dose, for example from about 3.2 milligram per kg body weight per dose to about 8 milligram per kg body weight per dose, such as from about 3.4 milligram per kg body weight per dose to about 8 milligram per kg body weight per dose, for example from about 3.6 milligram per kg body weight per dose to about 8 milligram per kg body weight per dose, such as from about 3.8 milligram per kg body weight per dose to about 8 milligram per kg body weight per dose, for example from about 4.0 milligram per kg body weight per dose to about 8 milligram per kg body weight per dose, such as from about 4.2 milligram per kg body weight per dose to about 8 milligram per kg body weight per dose, for example from about 4.4 milligram per kg body weight per dose to about 8 milligram per kg body weight per dose, such as from about 4.6 milligram per kg body weight per dose to about 8 milligram per kg body weight per dose, for example from about 4.8 milligram per kg body weight per dose to about 8 milligram per kg body weight per dose, such as from about 5.0 milligram per kg body weight per dose to about 8 milligram per kg body weight per dose, for example from about 5.2 milligram per kg body weight per dose to about 8 milligram per kg body weight per dose, such as from about 5.4 milligram per kg body weight per dose to about 8 milligram per kg body weight per dose, for example from about 5.6 milligram per kg body weight per dose to about 8 milligram per kg body weight per dose, such as from about 5.8 milligram per kg body weight per dose to about 8 milligram per kg body weight per dose, for example from about 6.0 milligram per kg body weight per dose to about 8 milligram per kg body weight per dose, such as from about 0.2 milligram per kg body weight per dose to about 6 milligram per kg body weight per dose, for example from about 0.3 milligram per kg body weight per dose to about 6 milligram per kg body weight per dose, such as from about 0.4 milligram per kg body weight per dose to about 6 milligram per kg body weight per dose, for example from about 0.5 milligram per kg body weight per dose to about 6 milligram per kg body weight per dose, such as from about 0.6 milligram per kg body weight per dose to about 6 milligram per kg body weight per dose, for example from about 0.7 milligram per kg body weight per dose to about 6 milligram per kg body weight per dose, such as from about 0.8 milligram per kg body weight per dose to about 6 milligram per kg body weight per dose, for example from about 0.9 milligram per kg body weight per dose to about 6 milligram per kg body weight per dose, such as from about 1.0 milligram per kg body weight per dose to about 6 milligram per kg body weight per dose, for example from about 1.2 milligram per kg body weight per dose to about 6 milligram per kg body weight per dose, such as from about 1.4 milligram per kg body weight per dose to about 6 milligram per kg body weight per dose, for example from about 1.6 milligram per kg body weight per dose to about 6 milligram per kg body weight per dose, such as from about 1.8 milligram per kg body weight per dose to about 6 milligram per kg body weight per dose, for example from about 2.0 milligram per kg body weight per dose to about 6 milligram per kg body weight per dose, such as from about 2.2 milligram per kg body weight per dose to about 6 milligram per kg body weight per dose, for example from about 2.4 milligram per kg body weight per dose to about 6 milligram per kg body weight per dose, such as from about 2.6 milligram per kg body weight per dose to about 6 milligram per kg body weight per dose, for example from about 2.8 milligram per kg body weight per dose to about 6 milligram per kg body weight per dose, such as from about 3.0 milligram per kg body weight per dose to about 6 milligram per kg body weight per dose, for example from about 3.2 milligram per kg body weight per dose to about 6 milligram per kg body weight per dose, such as from about 3.4 milligram per kg body weight per dose to about 6 milligram per kg body weight per dose, for example from about 3.6 milligram per kg body weight per dose to about 6 milligram per kg body weight per dose, such as from about 3.8 milligram per kg body weight per dose to about 6 milligram per kg body weight per dose, for example from about 4.0 milligram per kg body weight per dose to about 6 milligram per kg body weight per dose, such as from about 4.2 milligram per kg body weight per dose to about 6 milligram per kg body weight per dose, for example from about 4.4 milligram per kg body weight per dose to about 6 milligram per kg body weight per dose, such as from about 4.6 milligram per kg body weight per dose to about 6 milligram per kg body weight per dose, for example from about 4.8 milligram per kg body weight per dose to about 6 milligram per kg body weight per dose, such as from about 5.0 milligram per kg body weight per dose to about 6 milligram per kg body weight per dose.

Combination Therapy Employing Treatment with the Polypeptide According to the Present Invention Simultaneously with Another Drug

In the pharmaceutical treatment of pathological hemostasis, the use of more than one therapeutic compound simultaneously is common. This could enhance the anticoagulant activity while minimizing side effects from each of the administered compounds. Also, the time course in therapy is a major factor. An example of this is when heparin is given initially followed by warfarin, and when the effect of the latter commences, heparin administration ceases.

In the present invention, the simultaneous administration of the polypeptide described herein in the form of a pharmaceutical composition with at least another anticoagulant, antiplatelet or fibrinolytic compound is suggested. In one embodiment of the invention, the compound is given with at least 1 additional compound, such as 2 additional compounds, for example 3 additional compounds, such as 4 additional compounds, for example 5 additional compounds, such as 6 additional compounds or more.

These supplementary compounds include, but are not limited to the following bioactive agents: heparin, LMWH (bemiparin, dalteparin, enoxaparin, nadroparin, parnaparin, reviparin, tinzaparin), sulodexide, danaparoid (a combination of heparin sulphate, dermatan sulphate and chondroitin sulphate); warfarin and related coumarins and vitamin K antagonists (such as acenocoumarol, clorindione, coumatetralyl, dicumarol, diphenadione, ethyl biscoumacetate, phenprocoumon, phenindione and tioclomarol); the direct thrombin inhibitors including dabigatran, idraparinux, lepirudin, bivalirudin, argatroban, desirudin, hirudin, melagatran, ximelagatran and antithrombin III; factor Xa inhibitors such as rivaroxaban and fondaparinux; the anticoagulation factors such as protein C, protein S and TFPI; defibrotide and dermatan sulphate, the fibrinolytic agents such as streptokinase, tPA, uPA, urokinase, tenecteplase and anistreplase; serine endopeptidases ancrod, drotrecogin, fibrinolysin and brinase; and the anti-platelet agents including aspirin, aloxiprin, ditazole, carbasalate calcium, cloricromen, indobufen, picotamide, triflusal, clopidogrel, dipyridamole, prasugrel, ticlopidine, beraprost, prostacyclin, iloprost, treprostini and parenteral glycoprotein IIb/IIIa inhibitors such as abciximab, eptifibatide and tirofiban.

| | | The polypeptide claimed |
|---|---|---|
| Class of agent | Combination partners | herein |
| Anti-platelet | Aspirin | X |
| | aloxiprin | X |
| | ditazole | X |
| | Carbasalate calcium | X |
| | cloricromen | X |
| | indobufen | X |
| | picotamide | X |
| | triflusal | X |
| | clopidogrel | X |
| | dipyridamole | X |
| | prasugrel | X |
| | ticlopidine | X |
| | beraprost | X |
| | prostacyclin | X |
| | iloprost | X |
| | treprostini | X |
| | abciximab | X |
| | eptifibatide | X |
| | tirofiban | X |
| Anti-coagulant | Heparin | X |
| | bemiparin | X |
| | dalteparin | X |
| | enoxaparin | X |
| | nadroparin | X |
| | parnaparin | X |
| | reviparin | X |
| | tinzaparin | X |
| | sulodexide | X |
| | danaparoid | X |
| | Warfarin/coumarins | X |
| | Acenocoumarol | X |
| | Clorindione | X |
| | Coumatetralyl | X |
| | Dicumarol | X |
| | Diphenadione | X |
| | Ethyl biscoumacetate | X |
| | Phenprocoumon | X |
| | Phenindione | X |
| | Tioclomarol | X |
| | dabigatran | X |
| | idraparinux | X |
| | lepirudin | X |
| | bivalirudin | X |
| | Argatroban | X |
| | desirudin | X |
| | hirudin | X |
| | melagatran | X |
| | ximelagatran | X |
| | Antithrombin III | X |
| | rivaroxaban | X |
| | Fondaparinux | X |
| | protein C | X |
| | protein S | X |
| | TFPI | X |
| | Defibrotide | X |
| | Dermatan sulphate | X |
| Fibrinolytic | tenecteplase | X |
| | anistreplase | X |
| | ancrod | X |
| | drotrecogin | X |
| | fibrinolysin | X |
| | brinase | X |
| | tPA | X |
| | uPA | X |
| | urokinase | X |
| | streptokinase | X |

### Determination of Sequence Homologies and Identities

In one aspect the present invention provides isolated polypeptides that have a substantially similar sequence identity to the polypeptides according to the present invention, such as SEQ ID NO:1, or an ortholog thereof.

In another aspect the present invention provides isolated polypeptides that are different from SEQ ID NO:1 and have a substantially similar sequence identity to SEQ ID NO:1, or an ortholog thereof.

The term “substantially similar sequence identity” is used herein to denote polypeptides having at least 70%, such as at least 72%, for example at least 74%, such as at least 76%, for example at least 78%, such as at least 80%, for example at least 82%, such as at least 84%, for example at least 86%, such as at least 88%, for example at least 90%, such as at least 91%, for example at least 92%, such as at least 93%, for example at least 94%, such as at least 95%, for example at least 96%, such as at least 97%, for example at least 98%, such as at least 99%, or greater than 99% sequence identity to SEQ ID NO:1.

Additionally, the invention is embodied by the below listed items:

The polypeptide according to the present invention, wherein the polypeptide fragment contains less than 99.5%, such as less than 98%, e.g. less than 97%, such as less than 96%, e.g. less than 95%, such as less than 94%, e.g. less than 93%, such as less than 92%, e.g. less than 91%, such as less than 90%, e.g. less than 88%, such as less than 86%, e.g. less than 84%, e.g. less than 82%, such as less than 80%, e.g. less than 75%, such as less than 70%, e.g. less than 65%, such as less than 60%, e.g. less than 55%, such as less than 50%, e.g. less than 45%, such as less than 40%, e.g. less than 35%, such as less than 30%, e.g. less than 25%, such as less than 20%, such as less than 15%, e.g. less than 10% of the amino acid residues of SEQ ID NO:1.

The polypeptide according to the present invention, wherein the fragment contains less than 220 consecutive amino acid residues of SEQ ID NO:1, such as less than 215 consecutive amino acid residues, e.g. less than 210 consecutive amino acid residues, such as less than 205 consecutive amino acid residues, e.g. less than 200 consecutive amino acid residues, such as less than 195 consecutive amino acid residues, e.g. less than 190 consecutive amino acid residues, such as less than 185 consecutive amino acid residues, e.g. less than 180 consecutive amino acid residues, such as less than 175 consecutive amino acid residues, e.g. less than 170 consecutive amino acid residues, such as less than 165 consecutive amino acid residues, e.g. less than 160 consecutive amino acid residues, such as less than 155 consecutive amino acid residues, e.g. less than 150 consecutive amino acid residues, such as less than 145 consecutive amino acid residues, e.g. less than 140 consecutive amino acid residues, such as less than 135 consecutive amino acid residues, e.g. less than 130 consecutive amino acid residues, such as less than 125 consecutive amino acid residues, e.g. less than 120 consecutive amino acid residues, such as less than 115 consecutive amino acid residues, e.g. less than 110 consecutive amino acid residues, such as less than 105 consecutive amino acid residues, e.g. less than 100 consecutive amino acid residues, such as less than 95 consecutive amino acid residues, e.g. less than 90 consecutive amino acid residues, such as less than 85 consecutive amino acid residues, e.g. less than 80 consecutive amino acid residues, such as less than 75, e.g. less than 60, such as less than 45 consecutive amino acid residues of SEQ ID NO:1.

The polypeptide according to the present invention, wherein the fragment contains 6 or more consecutive amino acid residues, such as 7 or more consecutive amino acid residues, e.g. 8 or more consecutive amino acid residues, such as 9 or more consecutive amino acid residues, e.g. 10 or more consecutive amino acid residues, such as 12 or more consecutive amino acid residues, e.g. 14 or more consecutive amino acid residues, such as 16 or more consecutive amino acid residues, e.g. 18 or more consecutive amino acid residues, such as 20 or more consecutive amino acid residues, e.g. 22 or more consecutive amino acid residues, such as 24 or more consecutive amino acid residues, e.g. 26 or more consecutive amino acid residues, such as 28 or more consecutive amino acid residues, e.g. 30 or more consecutive amino acid residues of SEQ ID NO:1.

The polypeptide variant according to the present invention, wherein the polypeptide variant has at least 80% sequence identity, such as at least 81% sequence identity, e.g. at least 82% sequence identity, such as at least 83% sequence identity, e.g. at least 84% sequence identity, such as at least 85% sequence identity, e.g. at least 86% sequence identity, such as at least 87% sequence identity, e.g. at least 88% sequence identity, such as at least 89% sequence identity, e.g. at least 90% sequence identity, such as at least 91% sequence identity, e.g. at least 92% sequence identity, such as at least 93% sequence identity, e.g. at least 94% sequence identity, such as at least 95% sequence identity, e.g. at least 96% sequence identity, such as at least 97% sequence identity, e.g. at least 98% sequence identity, such as at least 99% sequence identity, e.g. at least 99.5% sequence identity to SEQ ID NO:1, or a fragment of SEQ ID NO:1.

The polypeptide according to the present invention, wherein the polypeptide variant fragment contains less than 99.5%, such as less than 98%, e.g. less than 97%, such as less than 96%, e.g. less than 95%, such as less than 94%, e.g. less than 93%, such as less than 92%, e.g. less than 91%, such as less than 90%, e.g. less than 88%, such as less than 86%, e.g. less than 84%, e.g. less than 82%, such as less than 80%, e.g. less than 75%, such as less than 70%, e.g. less than 65%, such as less than 60%, e.g. less than 55%, such as less than 50%, e.g. less than 45%, such as less than 40%, e.g. less than 35%, such as less than 30%, e.g. less than 25%, such as less than 20%, such as less than 15%, e.g. less than 10% of the amino acid residues of SEQ ID NO:1.

The polypeptide according to the present invention, wherein the polypeptide variant fragment contains less than 220 consecutive amino acid residues of SEQ ID NO:1, such as less than 215 consecutive amino acid residues, e.g. less than 210 consecutive amino acid residues, such as less than 205 consecutive amino acid residues, e.g. less than 200 consecutive amino acid residues, such as less than 195 consecutive amino acid residues, e.g. less than 190 consecutive amino acid residues, such as less than 185 consecutive amino acid residues, e.g. less than 180 consecutive amino acid residues, such as less than 175 consecutive amino acid residues, e.g. less than 170 consecutive amino acid residues, such as less than 165 consecutive amino acid residues, e.g. less than 160 consecutive amino acid residues, such as less than 155 consecutive amino acid residues, e.g. less than 150 consecutive amino acid residues, such as less than 145 consecutive amino acid residues, e.g. less than 140 consecutive amino acid residues, such as less than 135 consecutive amino acid residues, e.g. less than 130 consecutive amino acid residues, such as less than 125 consecutive amino acid residues, e.g. less than 120 consecutive amino acid residues, such as less than 115 consecutive amino acid residues, e.g. less than 110 consecutive amino acid residues, such as less than 105 consecutive amino acid residues, e.g. less than 100 consecutive amino acid residues, such as less than 95 consecutive amino acid residues, e.g. less than 90 consecutive amino acid residues, such as less than 85 consecutive amino acid residues, e.g. less than 80 consecutive amino acid residues, such as less than 75, e.g. less than 60, such as less than 45 consecutive amino acid residues of SEQ ID NO:1.

The polypeptide according to the present invention, wherein the polypeptide variant fragment contains 6 or more consecutive amino acid residues, such as 7 or more consecutive amino acid residues, e.g. 8 or more consecutive amino acid residues, such as 9 or more consecutive amino acid residues, e.g. 10 or more consecutive amino acid residues, such as 12 or more consecutive amino acid residues, e.g. 14 or more consecutive amino acid residues, such as 16 or more consecutive amino acid residues, e.g. 18 or more consecutive amino acid residues, such as 20 or more consecutive amino acid residues, e.g. 22 or more consecutive amino acid residues, such as 24 or more consecutive amino acid residues, e.g. 26 or more consecutive amino acid residues, such as 28 or more consecutive amino acid residues, e.g. 30 or more consecutive amino acid residues of SEQ ID NO:1.

The present invention also contemplates variant nucleic acid molecules that can be identified using two criteria: a) a determination of the identity or similarity between a polypeptide having the amino acid sequence of SEQ ID NO:1, cf above, and b) a hybridization assay carried out under stringent conditions. For example, certain gene variants comprise polynucleotides that remain hybridized with a polynucleotide encoding a polypeptide according to the present invention, such as SEQ ID NO:1, or a complement of such a polynucleotide, following washing under stringent washing conditions, in which the wash stringency is equivalent to 0.5× to 2×SSC with 0.1% SDS at 55° C. to 65° C. Alternatively, variant genes can be characterized as nucleic acid molecules that remain hybridized with a polynucleotide encoding a polypeptide according to the present invention, such as SEQ ID NO:1, or a complement of such a polynucleotide, following washing under stringent washing conditions, in which the wash stringency is equivalent to 0.1× to 0.2×SSC with 0.1% SDS at 55° C. to 65° C.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the longer sequence plus the number of gaps introduced into the longer sequence in order to align the two sequences])×(100).

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative or variant. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NO:1) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, and most preferably, three. The other parameters can be set as: gap opening penalty=10, and gap extension penalty=1.

### Substitution of Amino Acid Residues in Polypeptides According to the Present Invention

The present invention is also directed to polypeptides having one or more conservative amino acid substitution(s) and polynucleotides encoding polypeptides having one or more conservative amino acid substitution(s), as compared with the amino acid sequence of SEQ ID NO:1. That is, variants can be obtained that contain e.g. one or more amino acid substitutions of SEQ ID NO:1. Variants include sequences wherein an alkyl amino acid is substituted for an alkyl amino acid, wherein an aromatic amino acid is substituted for an aromatic amino acid, wherein a sulfur-containing amino acid is substituted for a sulfur-containing amino acid in, wherein a hydroxy-containing amino acid is substituted for a hydroxy-containing amino acid, wherein an acidic amino acid is substituted for an acidic amino acid, wherein a basic amino acid is substituted for a basic amino acid, or wherein a dibasic monocarboxylic amino acid is substituted for a dibasic monocarboxylic amino acid.

Among the common amino acids, for example, a “conservative amino acid substitution” can also be illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Particular variants of polypeptides are characterized by having at least 70%, at least 80%, at least 85%, at least 90%, at least 95% or greater than 95% sequence identity to a corresponding amino acid sequence disclosed herein (i.e., SEQ ID NO:1), e.g. when the variation in amino acid sequence is due to one or more conservative amino acid substitutions.

Variants of amino acid sequences, such as “conservative amino acid” variants, can be obtained, for example, by oligonucleotide-directed mutagenesis, linker-scanning mutagenesis, mutagenesis using the polymerase chain reaction, and the like (see Ausubel (1995) at pages 8 10 to 8 22; and McPherson (ed.), Directed Mutagenesis: A Practical Approach (IRL Press 1991)).

The polypeptides according to the present invention can also comprise non-naturally occurring amino acid residues. Non-naturally occurring amino acids include e.g., without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine.

Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is typically carried out in a cell-free system comprising an E. coli S30 extract and commercially available enzymes and other reagents. Proteins are purified by chromatography. See, for example, Robertson et al., J. Am. Chem. Soc. 113:2722 (1991), Ellman et al., Methods Enzymol. 202:301 (1991), Chung et al., Science 259:806 (1993), and Chung et al., Proc. Nat'l Acad. Sci. USA 90:10145 (1993).

Multiple amino acid substitutions can be made and tested using known methods of mutagenesis and screening, such as those disclosed by Reidhaar-Olson and Sauer (Science 241:53 (1988)) or Bowie and Sauer (Proc. Nat'l Acad. Sci. USA 86:2152 (1989)). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832 (1991), Ladner et al., U.S. Pat. No. 5,223,409, Huse, international publication No. WO 92/06204, and region-directed mutagenesis (Derbyshire et al., Gene 46:145 (1986), and Ner et al., DNA 7:127, (1988)).

Variants of the disclosed nucleotide and polypeptide sequences according to the present invention can also be generated through DNA shuffling as disclosed by Stemmer, Nature 370:389 (1994), Stemmer, Proc. Nat'l Acad. Sci. USA 91:10747 (1994), and international publication No. WO 97/20078. Briefly, variant DNA molecules are generated by in vitro homologous recombination by random fragmentation of a parent DNA followed by reassembly using PCR, resulting in randomly introduced point mutations. This technique can be modified by using a family of parent DNA molecules, such as allelic variants or DNA molecules from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode biologically active polypeptides, or polypeptides that bind specific antibodies, can be recovered from the host cells and rapidly sequenced using modern equipment. These methods allow the rapid determination of the importance of individual amino acid residues in a polypeptide of interest, and can be applied to polypeptides of unknown structure.

### Fragments of Polypeptides According to the Present Invention

The present invention also includes “functional fragments” of polypeptides and nucleic acid molecules according to the present invention encoding such functional fragments. Routine deletion analyses of nucleic acid molecules can be performed to obtain functional fragments of a nucleic acid molecule that encodes a polypeptide according to the present invention. As an illustration, DNA molecules encoding SEQ ID NO:1 can be digested with BaI31 nuclease to obtain a series of nested deletions. The fragments are then inserted into expression vectors in proper reading frame, and the expressed polypeptides are isolated and tested for the ability to bind specifically to anti-antibodies. One alternative to exonuclease digestion is to use oligonucleotide-directed mutagenesis to introduce deletions or stop codons to specify production of a desired fragment. Alternatively, particular fragments of a gene according to the present invention can be synthesized using the polymerase chain reaction.

Methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993), Content et al., “Expression and preliminary deletion analysis of the 42 kDa 2 5A synthetase induced by human interferon,” in Biological Interferon Systems, Proceedings of ISIR-TNO Meeting on Interferon Systems, Centell (ed.), pages 65 72 (Nijhoff 1987), Herschman, “The EGF Receptor,” in Control of Animal Cell Proliferation, Vol. 1, Boynton et al., (eds.) pages 169 199 (Academic Press 1985), Coumailleau et al., J. Biol. Chem. 270:29270 (1995); Fukunaga et al., J. Biol. Chem. 270:25291 (1995); Yamaguchi et al., Biochem. Pharmacol. 50:1295 (1995), and Meisel et al., Plant Molec. Biol. 30:1 (1996).

The present invention also contemplates functional fragments of a polypeptide according to the present invention that has one or more amino acid substitutions, compared with the amino acid sequence of e.g. SEQ ID NO:1. A variant polypeptide can be identified on the basis of structure by determining the level of identity with a particular amino acid sequence disclosed herein. An alternative approach to identifying a variant polypeptide on the basis of structure is to determine whether a nucleic acid molecule encoding a potential variant polypeptide can hybridize to a nucleic acid molecule encoding e.g. SEQ ID NO:1, as discussed above.

The present invention also provides polypeptide fragments or peptides comprising an epitope-bearing portion of a polypeptide according to the present invention as described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983)).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660 (1983)). Accordingly, antigenic epitope-bearing peptides and polypeptides of the present invention are useful to raise antibodies that bind with the polypeptides described herein.

Antigenic epitope-bearing peptides and polypeptides can contain at least 4 to 10 amino acids, for example at least 5 to 10 amino acids, such as at least 6 to 10 amino acids, for example at least 7 to 10 amino acids, such as at least 10 to 15 amino acids, for example about 15 to about 30 amino acids of e.g. SEQ ID NO:1.

Such epitope-bearing peptides and polypeptides can be produced by fragmenting a polypeptide according to the present invention, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105 116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60 84 (Cambridge University Press 1995), and Coligan et al. (eds.), Current Protocols in Immunology, pages 9.3.1 9.3.5 and pages 9.4.1 9.4.11 (John Wiley & Sons 1997).

Regardless of the particular nucleotide sequence of a variant gene according to the present invention, the gene encodes a polypeptide that may be characterized by its ability to bind specifically to an antibody capable of specifically binding e.g. to SEQ ID NO:1.

### Fusion Polypeptides

Fusion proteins comprising polypeptides according to the present invention can be used to express a polypeptide according to the present invention in a recombinant host, and to isolate expressed polypeptides. One type of fusion protein comprises a peptide that guides a polypeptide according to the present invention from a recombinant host cell. To direct a polypeptide according to the present invention into the secretory pathway of a eukaryotic host cell, a secretory signal sequence (also known as a signal peptide, a leader sequence, prepro sequence or pre sequence) is provided in a suitable expression vector. While the secretory signal sequence may be derived from a polypeptide according to the present invention, a suitable signal sequence may also be derived from another secreted protein or synthesized de novo. The secretory signal sequence is operably linked to a gene encoding sequence according to the present invention such that the two sequences are joined in the correct reading frame and positioned to direct the newly synthesized polypeptide into the secretory pathway of the host cell. Secretory signal sequences are commonly positioned 5′ to the nucleotide sequence encoding the polypeptide of interest, although certain secretory signal sequences may be positioned elsewhere in the nucleotide sequence of interest (see, e.g., Welch et al., U.S. Pat. No. 5,037,743; Holland et al., U.S. Pat. No. 5,143,830).

Although the secretory signal sequence of a gene according to the present invention, or another protein produced by mammalian cells (e.g., tissue-type plasminogen activator signal sequence, as described, for example, in U.S. Pat. No. 5,641,655) is useful for expression of a gene according to the present invention in recombinant mammalian hosts, a yeast signal sequence is preferred for expression in yeast cells. Examples of suitable yeast signal sequences are those derived from yeast mating phermone alpha-factor (encoded by the MF-alpha1 gene), invertase (encoded by the SUC2 gene), or acid phosphatase (encoded by the PHO5 gene). See, for example, Romanos et al., “Expression of Cloned Genes in Yeast,” in DNA Cloning 2: A Practical Approach, 2.sup.nd Edition, Glover and Hames (eds.), pages 123 167 (Oxford University Press 1995).

In bacterial cells, it is often desirable to express a heterologous protein as a fusion protein to decrease toxicity, increase stability, and to enhance recovery of the expressed protein. For example, a gene according to the present invention can be expressed as a fusion protein comprising a glutathione S-transferase polypeptide. Glutathione S-transferease fusion proteins are typically soluble, and easily purifiable from E. coli lysates on immobilized glutathione columns. In similar approaches, a fusion protein according to the present invention comprising a maltose binding protein polypeptide can be isolated with an amylose resin column, while a fusion protein comprising the C-terminal end of a truncated Protein A gene can be purified using IgG-Sepharose. Established techniques for expressing a heterologous polypeptide as a fusion protein in a bacterial cell are described, for example, by Williams et al., “Expression of Foreign Proteins in E. coli Using Plasmid Vectors and Purification of Specific Polyclonal Antibodies,” in DNA Cloning 2: A Practical Approach, 2.sup.nd Edition, Glover and Hames (Eds.), pages 15 58 (Oxford University Press 1995). In addition, commercially available expression systems are available. For example, the PINPOINT Xa protein purification system (Promega Corporation; Madison, Wis.) provides a method for isolating a fusion protein comprising a polypeptide that becomes biotinylated during expression with a resin that comprises avidin.

Peptide tags that are useful for isolating heterologous polypeptides expressed by either prokaryotic or eukaryotic cells include polyHistidine tags (which have an affinity for nickel-chelating resin), c-myc tags, calmodulin binding protein (isolated with calmodulin affinity chromatography), substance P, the RYIRS tag (which binds with anti-RYIRS antibodies), the Glu-Glu tag, and the FLAG tag (which binds with anti-FLAG antibodies). See, for example, Luo et al., Arch. Biochem. Biophys. 329:215 (1996), Morganti et al., Biotechnol. Appl. Biochem. 23:67 (1996), and Zheng et al., Gene 186:55 (1997). Nucleic acid molecules encoding such peptide tags are available, for example, from Sigma-Aldrich Corporation (St. Louis, Mo.).

Another form of fusion protein comprises a polypeptide according to the present invention and an immunoglobulin heavy chain constant region, typically an F_{c} fragment, which contains two constant region domains and a hinge region but lacks the variable region. As an illustration, Chang et al., U.S. Pat. No. 5,723,125, describe a fusion protein comprising a human interferon and a human immunoglobulin Fc fragment. The C-terminal of the interferon is linked to the N-terminal of the Fc fragment by a peptide linker moiety. An example of a peptide linker is a peptide comprising primarily a T cell inert sequence, which is immunologically inert. An exemplary peptide linker has the amino acid sequence: GGSGG SGGGG SGGGG S (SEQ ID NO:13). In this fusion protein, a preferred F_{c} moiety is a human gamma4 chain, which is stable in solution and has little or no complement activating activity. Accordingly, the present invention contemplates a fusion protein that comprises a polypeptide according to the present invention, or a fragment thereof, and a human F_{c} fragment, wherein the C-terminus of the polypeptide according to the present invention, or a fragment thereof, is attached to the N-terminus of the F_{c} fragment via a peptide linker.

In another variation, a fusion protein comprising a polypeptide according to the present invention further comprises an IgG sequence. The polypeptide moiety according to the present invention is covalently joined to the amino terminal end of the IgG sequence, and a signal peptide that is covalently joined to the amino terminal of the polypeptide moiety according to the present invention, wherein the IgG sequence comprises or consists of the following elements in the following order: a hinge region, a CH₂ domain, and a CH₃ domain. Accordingly, the IgG sequence lacks a CH₁ domain. The polypeptide moiety according to the present invention displays a protease inhibiting activity. The above, general approaches for producing fusion proteins that comprise both antibody and nonantibody portions has been described by LaRochelle et al., EP 742830 (WO 95/21258).

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. General methods for enzymatic and chemical cleavage of fusion proteins are described, for example, by Ausubel (1995) at pages 16 19 to 16 25.

### General Methods for the Production of Polypeptides According to the Present Invention

The polypeptides of the present invention, including full-length polypeptides, functional fragments, and fusion proteins, can be produced in recombinant host cells following conventional techniques. To express a gene according to the present invention, a nucleic acid molecule encoding the polypeptide must be operably linked to regulatory sequences that control transcriptional expression in an expression vector and then introduced into a host cell. In addition to transcriptional regulatory sequences, such as promoters and enhancers, expression vectors can include translational regulatory sequences and a marker gene, which is suitable for selection of cells that carry the expression vector.

The host cells which may comprise the polypeptide according to the invention can be exemplified by animal cells, mammalian cells, insect cells, fungal cells, yeast cells, bacterial cells and plant cells. In a particular embodiment the host cell is a mammalian cell, such as African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 [Chasin et al., Som. Cell. Molec. Genet. 12:555 1986]), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658).

Expression vectors that are suitable for production of a foreign protein in eukaryotic cells typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence. As discussed above, expression vectors can also include nucleotide sequences encoding a secretory sequence that directs the heterologous polypeptide into the secretory pathway of a host cell. For example, an expression vector may comprise a gene according to the present invention and a secretory sequence derived from said gene or another secreted gene.

Examples of vectors commonly used with bacteria include the pET series (Novagen), pGEX series (Ge Healthcare), pBAD-series (Invitrogen). Examples of vectors in yeasts are the pPic series for Pichia (Invitrogen), the pKIac system from Kluyveromyces lactis (New England biolabs), S. cereviseae vectors (Patel, O., Fearnley, R., and Macreadie, I. 3002. Saccharomyces cerevisiae expression vectors with thrombin-cleavable N- and C-terminal 6×(His) tags. Biotechnol Lett. 2003 25(4):331-334) and the pYes system for S. cereviseae (Invitrogen). Examples of vectors for use in funghi are the pBAR series (described in Pall, M. L. and J. Brunelli. 1993. A series of six compact fungal transformation vectors containing polylinkers with unique restrictions sites. Fungal Genetics Newsletter 40: 59-61). The pIEx plasmid based system (Merck) or the baculovirus based system (Merck) are two examples of systems useful for insect cells. Similar products are available from other companies.

Examples of vectors for use in insect cells include the tetracycline regulated systems pTet and pTre, the adenovirus-based system Adeno-X, the retrovirus-based system Rethro-X (all Clontech) and the pcDNA vectors (Invitrogen). Again, many more examples exist and are on the market.

Polypeptides according to the present invention may be expressed in mammalian cells. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 [Chasin et al., Som. Cell. Molec. Genet. 12:555 1986]), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658).

Mammalian hosts cells used for expressing the polypeptides according to the present invention is by no means intended as a process for modifying the germ line genetic identity of human beings, as only immortalized or transformed diploid human cells are described in this process.

For a mammalian host, the transcriptional and translational regulatory signals may be derived from viral sources, such as adenovirus, bovine papilloma virus, simian virus, or the like, in which the regulatory signals are associated with a particular gene which has a high level of expression. Suitable transcriptional and translational regulatory sequences also can be obtained from mammalian genes, such as actin, collagen, myosin, and metallothionein genes.

Transcriptional regulatory sequences include a promoter region sufficient to direct the initiation of RNA synthesis. Suitable eukaryotic promoters include the promoter of the mouse metallothionein I gene (Hamer et al., J. Molec. Appl. Genet. 1:273 (1982)), the TK promoter of Herpes virus (McKnight, Cell 31:355 (1982)), the SV40 early promoter (Benoist et al., Nature 290:304 (1981)), the Rous sarcoma virus promoter (Gorman et al., Proc. Nat'l Acad. Sci. USA 79:6777 (1982)), the cytomegalovirus promoter (Foecking et al., Gene 45:101 (1980)), and the mouse mammary tumor virus promoter (see, generally, Etcheverry, “Expression of Engineered Proteins in Mammalian Cell Culture,” in Protein Engineering: Principles and Practice, Cleland et al. (eds.), pages 163 181 (John Wiley & Sons, Inc. 1996)).

Alternatively, a prokaryotic promoter, such as the bacteriophage T3 RNA polymerase promoter, can be used to control gene expression in mammalian cells if the prokaryotic promoter is regulated by a eukaryotic promoter (Zhou et al., Mol. Cell. Biol. 10:4529 (1990), and Kaufman et al., Nucl. Acids Res. 19:4485 (1991)).

An expression vector can be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

For example, one suitable selectable marker is a gene that provides resistance to the antibiotic neomycin. In this case, selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A suitable amplifiable selectable marker is dihydrofolate reductase, which confers resistance to methotrexate. Other drug resistance genes (e.g., hygromycin resistance, multi-drug resistance, puromycin acetyltransferase) can also be used. Alternatively, markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Polypeptides according to the present invention can also be produced by cultured mammalian cells using a viral delivery system. Exemplary viruses for this purpose include adenovirus, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161 (1994), and Douglas and Curiel, Science & Medicine 4:44 (1997)). Advantages of the adenovirus system include the accommodation of relatively large DNA inserts, the ability to grow to high-titer, the ability to infect a broad range of mammalian cell types, and flexibility that allows use with a large number of available vectors containing different promoters.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. An option is to delete the essential E1 gene from the viral vector, which results in the inability to replicate unless the E1 gene is provided by the host cell. Adenovirus vector-infected human 293 cells (ATCC Nos. CRL-1573, 45504, 45505), for example, can be grown as adherent cells or in suspension culture at relatively high cell density to produce significant amounts of protein (see Garnier et al., Cytotechnol. 15:145 (1994)).

The baculovirus system provides an efficient means to introduce cloned genes according to the present invention into insect cells. Suitable expression vectors are based upon the Autographa californica multiple nuclear polyhedrosis virus (AcMNPV), and contain well-known promoters such as Drosophila heat shock protein (hsp) 70 promoter, Autographa californica nuclear polyhedrosis virus immediate-early gene promoter (ie-1) and the delayed early 39K promoter, baculovirus p10 promoter, and the Drosophila metallothionein promoter. A second method of making recombinant baculovirus utilizes a transposon-based system described by Luckow (Luckow, et al., J. Virol. 67:4566 (1993)). This system, which utilizes transfer vectors, is sold in the BAC-to-BAC kit (Life Technologies, Rockville, Md.). This system utilizes a transfer vector, PFASTBAC (Life Technologies) containing a Tn7 transposon to move the DNA encoding the polypeptide according to the present invention into a baculovirus genome maintained in E. coli as a large plasmid called a “bacmid.” See, Hill-Perkins and Possee, J. Gen. Virol. 71:971 (1990), Bonning, et al., J. Gen. Virol. 75:1551 (1994), and Chazenbalk, and Rapoport, J. Biol. Chem. 270:1543 (1995). In addition, transfer vectors can include an in-frame fusion with DNA encoding an epitope tag at the C- or N-terminus of the expressed polypeptide according to the present invention, for example, a Glu-Glu epitope tag (Grussenmeyer et al., Proc. Nat'l Acad. Sci. 82:7952 (1985)). Using a technique known in the art, a transfer vector containing a gene according to the present invention is transformed into E. coli, and screened for bacmids, which contain an interrupted lacZ gene indicative of recombinant baculovirus. The bacmid DNA containing the recombinant baculovirus genome is then isolated using common techniques.

The illustrative PFASTBAC vector can be modified to a considerable degree. For example, the polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins (see, for example, Hill-Perkins and Possee, J. Gen. Virol. 71:971 (1990), Bonning, et al., J. Gen. Virol. 75:1551 (1994), and Chazenbalk and Rapoport, J. Biol. Chem. 270:1543 (1995). In such transfer vector constructs, a short or long version of the basic protein promoter can be used. Moreover, transfer vectors can be constructed which replace the native secretory signal sequences of polypeptides according to the present invention with secretory signal sequences derived from insect proteins. For example, a secretory signal sequence from Ecdysteroid Glucosyltransferase (EGT), honey bee Melittin (Invitrogen Corporation; Carlsbad, Calif.), or baculovirus gp67 (PharMingen: San Diego, Calif.) can be used in constructs to replace native secretory signal sequences.

The recombinant virus or bacmid is used to transfect host cells. Suitable insect host cells include cell lines derived from IPLB-Sf-21, a Spodoptera frugiperda pupal ovarian cell line, such as Sf9 (ATCC CRL 1711), Sf21AE, and Sf21 (Invitrogen Corporation; San Diego, Calif.), as well as Drosophila Schneider-2 cells, and the HIGH FIVEO cell line (Invitrogen) derived from Trichoplusia ni (U.S. Pat. No. 5,300,435). Commercially available serum-free media can be used to grow and to maintain the cells. Suitable media are Sf900 II™ (Life Technologies) or ESF 921™ (Expression Systems) for Sf9 cells; and Ex-cellO405™ (JRH Biosciences, Lenexa, Kans.) or Express FiveO™ (Life Technologies) for T. ni cells. When recombinant virus is used, the cells are typically grown up from an inoculation density of approximately 2 to 5×10⁵ cells to a density of 1 to 2×10⁶ cells at which time a recombinant viral stock is added at a multiplicity of infection (MOI) of 0.1 to 10, more typically near 3.

Established techniques for producing recombinant proteins in baculovirus systems are provided by Bailey et al., “Manipulation of Baculovirus Vectors,” in Methods in Molecular Biology, Volume 7: Gene Transfer and Expression Protocols, Murray (ed.), pages 147 168 (The Humana Press, Inc. 1991), by Patel et al., “The baculovirus expression system,” in DNA Cloning 2: Expression Systems, 2nd Edition, Glover et al. (eds.), pages 205 244 (Oxford University Press 1995), by Ausubel (1995) at pages 16 37 to 16 57, by Richardson (ed.), Baculovirus Expression Protocols (The Humana Press, Inc. 1995), and by Lucknow, “Insect Cell Expression Technology,” in Protein Engineering: Principles and Practice, Cleland et al. (eds.), pages 183 218 (John Wiley & Sons, Inc. 1996).

Fungal cells, including yeast cells, can also be used to express the genes described herein. Yeast species of particular interest in this regard include Saccharomyces cerevisiae, Pichia pastoris, and Pichia methanolica. Suitable promoters for expression in yeast include promoters from GAL1 (galactose), PGK (phosphoglycerate kinase), ADH (alcohol dehydrogenase), AOX1 (alcohol oxidase), HIS4 (histidinol dehydrogenase), and the like. Many yeast cloning vectors have been designed and are readily available. These vectors include YIp-based vectors, such as YIp5, YRp vectors, such as YRp17, YEp vectors such as YEp13 and YCp vectors, such as YCp19. Methods for transforming S. cerevisiae cells with exogenous DNA and producing recombinant polypeptides therefrom are disclosed by, for example, Kawasaki, U.S. Pat. No. 4,599,311, Kawasaki et al., U.S. Pat. No. 4,931,373, Brake, U.S. Pat. No. 4,870,008, Welch et al., U.S. Pat. No. 5,037,743, and Murray et al., U.S. Pat. No. 4,845,075. Transformed cells are selected by phenotype determined by the selectable marker, commonly drug resistance or the ability to grow in the absence of a particular nutrient (e.g., leucine). A suitable vector system for use in Saccharomyces cerevisiae is the POT1 vector system disclosed by Kawasaki et al. (U.S. Pat. No. 4,931,373), which allows transformed cells to be selected by growth in glucose-containing media. Additional suitable promoters and terminators for use in yeast include those from glycolytic enzyme genes (see, e.g., Kawasaki, U.S. Pat. No. 4,599,311, Kingsman et al., U.S. Pat. No. 4,615,974, and Bitter, U.S. Pat. No. 4,977,092) and alcohol dehydrogenase genes. See also U.S. Pat. Nos. 4,990,446, 5,063,154, 5,139,936, and 4,661,454. Other examples of commonly used and/or commercially available vectors suitable for use in yeast are the pPic series (Invitrogen), the pKIac system from Kluyveromyces lactis (New England Biolabs) and S. cerevisae vectors (Patel et al., Biotechnology letters 2003 vol 25(4):331-334) as well as the pYes system for S. cerevisae (Invitrogen). In fungi, the pBAR series is useful (Pall et al., 1993 vol. 40:59-61, Functional Genetics Newsletter).

Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459 (1986), and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

For example, the use of Pichia methanolica as host for the production of recombinant proteins is disclosed by Raymond, U.S. Pat. No. 5,716,808, Raymond, U.S. Pat. No. 5,736,383, Raymond et al., Yeast 14:11 23 (1998), and in international publication Nos. WO 97/17450, WO 97/17451, WO 98/02536, and WO 98/02565. DNA molecules for use in transforming P. methanolica will commonly be prepared as double-stranded, circular plasmids, which can be linearized prior to transformation. For polypeptide production in P. methanolica, the promoter and terminator in the plasmid can be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A suitable selectable marker for use in Pichia methanolica is a P. methanolica ADE2 gene, which encodes phosphoribosyl-5-aminoimidazole carboxylase (AIRC; EC 4.1.1.21), and which allows ade2 host cells to grow in the absence of adenine. For large-scale, industrial processes where it is desirable to minimize the use of methanol, it is possible to use host cells in which both methanol utilization genes (AUG1 and AUG2) are deleted. For production of secreted proteins, host cells can be used that are deficient in vacuolar pro tease genes (PEP4 and PRB1). Electroporation is used to facilitate the introduction of a plasmid containing DNA encoding a polypeptide of interest into P. methanolica cells. P. methanolica cells can be transformed by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Expression vectors can also be introduced into plant protoplasts, intact plant tissues, or isolated plant cells. Methods for introducing expression vectors into plant tissue include the direct infection or co-cultivation of plant tissue with Agrobacterium tumefaciens, microprojectile-mediated delivery, DNA injection, electroporation, and the like. See, for example, Horsch et al., Science 227:1229 (1985), Klein et al., Biotechnology 10:268 (1992), and Miki et al., “Procedures for Introducing Foreign DNA into Plants,” in Methods in Plant Molecular Biology and Biotechnology, Glick et al. (eds.), pages 67 88 (CRC Press, 1993).

Alternatively, genes according to the present invention can be expressed in prokaryotic host cells. Suitable promoters that can be used to express polypeptides according to the present invention in a prokaryotic host are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, Ipp-lacSpr, phoA, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. Prokaryotic promoters have been reviewed by Glick, J. Ind. Microbiol. 1:277 (1987), Watson et al., Molecular Biology of the Gene, 4th Ed. (Benjamin Cummins 1987), and by Ausubel et al. (1995).

Suitable prokaryotic hosts include E. coli and Bacillus subtilus. Suitable strains of E. coli include BL21(DE3), BL21(DE3)pLysS, BL21(DE3)pLysE, DH1, DH41, DH5, DH5I, DH5IF, DH5IMCR, DH10B, DH10B/p3, DH11S, C600, HB101, JM101, JM105, JM109, JM110, K38, RR1, Y1088, Y1089, CSH18, ER1451, and ER1647 (see, for example, Brown (ed.), Molecular Biology Labfax (Academic Press 1991)). Suitable strains of Bacillus subtilus include BR151, YB886, MI119, MI120, and B170 (see, for example, Hardy, “Bacillus Cloning Methods,” in DNA Cloning: A Practical Approach, Glover (ed.) (IRL Press 1985)).

When expressing a polypeptide according to the present invention in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Methods for expressing proteins in prokaryotic hosts are well-known to those of skill in the art (see, for example, Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995), Ward et al., “Genetic Manipulation and Expression of Antibodies,” in Monoclonal Antibodies: Principles and Applications, page 137 (Wiley-Liss, Inc. 1995), and Georgiou, “Expression of Proteins in Bacteria,” in Protein Engineering: Principles and Practice, Cleland et al. (eds.), page 101 (John Wiley & Sons, Inc. 1996)).

Standard methods for introducing expression vectors into bacterial, yeast, insect, and plant cells are provided, for example, by Ausubel (1995).

General methods for expressing and recovering foreign protein produced by a mammalian cell system are provided by, for example, Etcheverry, “Expression of Engineered Proteins in Mammalian Cell Culture,” in Protein Engineering: Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2: Expression Systems, 2nd Edition, Glover et al. (eds.), pages 59 92 (Oxford University Press 1995). Established methods for isolating recombinant proteins from a baculovirus system are described by Richardson (ed.), Baculovirus Expression Protocols (The Humana Press, Inc. 1995).

As an alternative, polypeptides of the present invention can be synthesized by exclusive solid phase synthesis, partial solid phase methods, fragment condensation or classical solution synthesis. These synthesis methods are well-known to those of skill in the art (see, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept. Prot. 3:3 (1986), Atherton et al., Solid Phase Peptide Synthesis: A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, for example, Dawson et al., Science 266:776 (1994), Hackeng et al., Proc. Nat'l Acad. Sci. USA 94:7845 (1997), Dawson, Methods Enzymol. 287: 34 (1997), Muir et al, Proc. Nat'l Acad. Sci. USA 95:6705 (1998), and Severinov and Muir, J. Biol. Chem. 273:16205 (1998)).

The present invention contemplates compositions comprising a peptide or polypeptide described herein. Such compositions can further comprise a carrier. The carrier can be a conventional organic or inorganic carrier. Examples of carriers include water, buffer solution, alcohol, propylene glycol, macrogol, sesame oil, corn oil, and the like.

### Isolation of Polypeptides According to the Present Invention

The polypeptides of the present invention can be purified to at least about 80% purity, to at least about 90% purity, to at least about 95% purity, or even greater than 95% purity with respect to contaminating macromolecules, particularly other proteins and nucleic acids, and free of infectious and pyrogenic agents. The polypeptides of the present invention can also be purified to a pharmaceutically pure state, which is greater than 99.9% pure. In certain preparations, a purified polypeptide is substantially free of other polypeptides, particularly other polypeptides of animal origin.

Fractionation and/or conventional purification methods can be used to obtain preparations of polypeptides according to the present invention purified from natural sources, and recombinant polypeptides according to the present invention and fusion polypeptides according to the present invention purified from recombinant host cells. In general, ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties.

Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Selection of a particular method for polypeptide isolation and purification is a matter of routine design and is determined in part by the properties of the chosen support. See, for example, Affinity Chromatography: Principles & Methods (Pharmacia LKB Biotechnology 1988), and Doonan, Protein Purification Protocols (The Humana Press 1996).

Additional variations in the isolation and purification of polypeptides according to the present invention can be devised by those of skill in the art. For example, specific antibodies recognising polypeptides according to the present invention and fragments thereof, obtained as described below, can be used to isolate large quantities of protein by immunoaffinity purification.

The polypeptides of the present invention can also be isolated by exploitation of particular properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1 (1985)). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (M. Deutscher, (ed.), Meth. Enzymol. 182:529 (1990)). Within additional embodiments of the invention, a fusion of the polypeptide of interest and an affinity tag (e.g., maltose-binding protein, an immunoglobulin domain) may be constructed to facilitate purification.

Polypeptides and fragments thereof according to the present invention may also be prepared through chemical synthesis, as described above. Polypeptides according to the present invention may be monomers or multimers; glycosylated or non-glycosylated; pegylated or non-pegylated; and may or may not include an initial methionine amino acid residue.

### Production of Antibodies Specific for Polypeptides According to the Present Invention

Antibodies to a polypeptide according to the present invention, or a fragment thereof, can be obtained, for example, by using as an antigen the product produced from an expression vector comprising a gene according to the present invention in a suitable host organism, or by using a polypeptide according to the present invention isolated from a natural source or synthesised using any conventional solid phase synthesis strategy. Particularly useful antibodies “bind specifically” with a polypeptide according to the present invention. Antibodies are considered to be specifically binding if the antibodies exhibit at least one of the following two properties: (1) antibodies bind to a polypeptide according to the present invention with a threshold level of binding activity, and (2) antibodies do not significantly cross-react with polypeptides which are related to a polypeptide according to the present invention as defined herein below.

With regard to the first characteristic, antibodies specifically bind if they bind to a polypeptide, peptide or epitope with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660 (1949)). With regard to the second characteristic, antibodies do not significantly cross-react with related polypeptide molecules, for example, if they detect polypeptides according to the present invention, but do not detect known polypeptides applied in similar or identical amounts in a standard Western blot analysis.

Antibodies can be produced using antigenic epitope-bearing peptides or polypeptides according to the present invention. Antigenic epitope-bearing peptides and polypeptides of the present invention preferably contain a sequence of at least four, or between 15 to about 30 amino acids contained within SEQ ID NO:1. However, peptides or polypeptides comprising a larger portion of an amino acid sequence of the invention, containing from 30 to 50 amino acids, or any length up to and including the entire amino acid sequence of a polypeptide according to the invention, also are useful for inducing antibodies that bind with polypeptides according to the present invention. It is desirable that the amino acid sequence of the epitope-bearing peptide is selected to provide substantial solubility in aqueous solvents (i.e., the sequence includes relatively hydrophilic residues, while hydrophobic residues are preferably avoided). Moreover, amino acid sequences containing proline residues may be also be desirable for antibody production.

As an illustration, potential antigenic sites in polypeptides according to the present invention can be identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

The Jameson-Wolf method predicts potential antigenic determinants by combining six major subroutines for protein structural prediction. Briefly, the Hopp-Woods method, Hopp et al., Proc. Nat'l Acad. Sci. USA 78:3824 (1981), was first used to identify amino acid sequences representing areas of greatest local hydrophilicity (parameter: seven residues averaged). In the second step, Emini's method, Emini et al., J. Virology 55:836 (1985), was used to calculate surface probabilities (parameter: surface decision threshold (0.6)=1). Third, the Karplus-Schultz method, Karplus and Schultz, Naturwissenschaften 72:212 (1985), was used to predict backbone chain flexibility (parameter: flexibility threshold (0.2)=1). In the fourth and fifth steps of the analysis, secondary structure predictions were applied to the data using the methods of Chou-Fasman, Chou, “Prediction of Protein Structural Classes from Amino Acid Composition,” in Prediction of Protein Structure and the Principles of Protein Conformation, Fasman (ed.), pages 549 586 (Plenum Press 1990), and Garnier-Robson, Garnier et al., J. Mol. Biol. 120:97 (1978) (Chou-Fasman parameters: conformation table=64 proteins; .alpha. region threshold=103; .beta. region threshold=105; Garnier-Robson parameters: .alpha. and .beta. decision constants=0). In the sixth subroutine, flexibility parameters and hydropathy/solvent accessibility factors were combined to determine a surface contour value, designated as the “antigenic index.” Finally, a peak broadening function was applied to the antigenic index, which broadens major surface peaks by adding 20, 40, 60, or 80% of the respective peak value to account for additional free energy derived from the mobility of surface regions relative to interior regions. This calculation was not applied, however, to any major peak that resides in a helical region, since helical regions tend to be less flexible.

Polyclonal antibodies to recombinant protein or isolated from natural sources can be prepared using methods well-known to those of skill in the art. See, for example, Green et al., “Production of Polyclonal Antisera,” in Immunochemical Protocols (Manson, ed.), pages 1 to 5 (Humana Press 1992), and Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995). The immunogenicity of a polypeptide can be increased through the use of an adjuvant, such as alum (aluminum hydroxide) or Freund's complete or incomplete adjuvant. Polypeptides useful for immunization also include fusion polypeptides, such as fusions of or a portion thereof with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, guinea pigs, goats, or sheep, an antibody specific for a polypeptides according to the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310 (1990).

Alternatively, monoclonal antibodies specific for a polypeptides according to the present invention can be generated. Rodent monoclonal antibodies to specific antigens may be obtained by methods known to those skilled in the art (see, for example, Kohler et al., Nature 256:495 (1975), Coligan et al. (eds.), Current Protocols in Immunology, Vol. 1, pages 2.5.1 2.6.7 (John Wiley & Sons 1991) [“Coligan”], Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice with a composition comprising a gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

In addition, an antibody specific for polypeptides according to the present invention of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13 (1994), Lonberg et al., Nature 368:856 (1994), and Taylor et al., Int. Immun. 6:579 (1994).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1 2.7.12 and pages 2.9.1 2.9.3; Baines et al., “Purification of Immunoglobulin G (IgG),” in Methods in Molecular Biology, Vol. 10, pages 79 104 (The Humana Press, Inc. 1992)).

For particular uses, it may be desirable to prepare fragments of antibodies specific for polypeptides according to the present invention. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an F_{c} fragment directly. These methods are described, for example, by Goldenberg, U.S. Pat. No. 4,331,647, Nisonoff et al., Arch Biochem. Biophys. 89:230 (1960), Porter, Biochem. J. 73:119 (1959), Edelman et al. and Coligan, both in Methods in Enzymology Vol. 1, (Academic Press 1967).

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Nat'l Acad. Sci. USA 69:2659 (1972). Alternatively, the variable chains can be linked by an intermolecular disulfide bond or cross-linked by chemicals such as glutaraldehyde (see, for example, Sandhu, Crit. Rev. Biotech. 12:437 (1992)).

The Fv fragments may comprise V_{H} and V_{L} chains, which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97 (1991) (also see, Bird et al., Science 242:423 (1988), Ladner et al., U.S. Pat. No. 4,946,778, Pack et al., Bio/Technology 11:1271 (1993), and Sandhu, supra).

As an illustration, a scFV can be obtained by exposing lymphocytes to polypeptide in vitro, and selecting antibody display libraries in phage or similar vectors (for instance, through use of immobilized or labeled protein or peptide). Genes encoding polypeptides having potential polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides, which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from CLONTECH Laboratories, Inc. (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the sequences disclosed herein to identify proteins which bind to.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106 (1991), Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an antibody specific for a polypeptide according to the present invention may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989). Techniques for producing humanized monoclonal antibodies are described, for example, by Jones et al., Nature 321:522 (1986), Carter et al., Proc. Nat'l Acad. Sci. USA 89:4285 (1992), Sandhu, Crit. Rev. Biotech. 12:437 (1992), Singer et al., J. Immun. 150:2844 (1993), Sudhir (ed.), Antibody Engineering Protocols (Humana Press, Inc. 1995), Kelley, “Engineering Therapeutic Antibodies,” in Protein Engineering: Principles and Practice, Cleland et al. (eds.), pages 399 434 (John Wiley & Sons, Inc. 1996), and by Queen et al., U.S. Pat. No. 5,693,762 (1997).

Polyclonal anti-idiotype antibodies can be prepared by immunizing animals with antibodies or antibody fragments specific for a polypeptide according to the present invention, using standard techniques. See, for example, Green et al., “Production of Polyclonal Antisera,” in Methods In Molecular Biology: Immunochemical Protocols, Manson (ed.), pages 1 12 (Humana Press 1992). Also, see Coligan at pages 241 to 247. Alternatively, monoclonal anti-idiotype antibodies can be prepared using antibodies or antibody fragments specific for a polypeptide according to the present invention as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677, and Varthakavi and Minocha, J. Gen. Virol. 77:1875 (1996).

One aspect of the present invention relates to a method for inhibiting Factor X activity comprising the steps of administering a polypeptide according to the present invention to an individual in need thereof and inhibiting Factor X in the individual.

Furthermore, the present invention relates to a method for reversing the inhibition of Factor X by administration of one or more antibodies that recognizes a site wherein the polypeptide of the present invention inhibits Factor X, binding of said one or more antibodies to the polypeptide, at least partly dissociating the polypeptide from Factor X, thereby reversing the inhibition of Factor X activity. In one embodiment the antibody is polyclonal, however, in a particular embodiment the antibody is monoclonal.

### Coating Composition and Medico-Technological Devices

One aspect of the present invention relates to a coating composition comprising the polypeptide of the present invention. Such a coating composition may be used to coat for example medico-technological devices. In one embodiment the coating composition of the present invention is a solution comprising the polypeptide of the present invention. Therefore in another aspect the present invention relates to a medico-technological device coated with the coating composition comprising the polypeptide of the present invention.

The medico-technological device to be coated with the coating composition or coated with the coating composition of the present invention include all devices, instruments, structures, etc. intended to be in contact with at least one mammalian body fluid. A “medico-technological device”, as used herein, thus refers to a device having surfaces that contact tissue, blood, or other bodily fluids of a mammal, in particular humans, in the course of their operation or utility. Examples of such medico-technological devices include i) extracorporeal devices for use in surgery such as blood oxygenators, blood pumps, blood sensors, tubing used to carry blood and the like which contact blood which is then returned to the mammal; (ii) prostheses implanted in a human or animal body such as vascular grafts, stents, pacemaker leads, heart valves, and the like that are implanted in blood vessels or in the heart; (iii) devices for temporary intravascular use such as catheters, guide wires, and the like which are placed into blood vessels or the heart for purposes of monitoring or repair.

The devices can be prepared by coating the exposed surface in part or completely with the coating composition of the present invention. For example, this can be done by submersing the device into the coating composition of the present invention and then allowing excess coating composition to drain from the device. Alternately, the coating may be applied by spraying techniques, dipping techniques and other techniques that allow the coating composition to come into contact with the device or device surface. The coating may then be dried in an appropriate atmosphere (low humidity, temperature-controlled, dust-free, and sterile if aseptic processing is required).

The medico-technological devices may be made of a variety of metals, including stainless steel and platinum. However, the medico-technological devices may also be made of plastic.

Examples of medico-technological devices that can be coated using the coating composition of the present invention are: plastic and metal tubing(s), plastic and metal catheters, plastic and metal cannulas and needles or needle-assemblies, surgical instruments such as clamps, forceps, retractors, etc., sutures, plastic (such as polyethylene) strips, meshes, stents and slings. The utility of the present invention extends to other devices not specifically listed here that may be partially or fully coated using the formulations suggested.

Examples of medico-technological devices are suture fastener devices that are typically used to reattach tissue to bone. Often the procedures involve the attachment of tendon, ligament, or other soft tissue to bones in the shoulder, knee, elbow, wrist, hand, and ankle. In one approach, bone anchors are inserted into the bone and then soft tissue such as ligament or tendon may be sutured to the anchor point

Other examples of medico-technological devices are screws, for example biodegradable screws, including interference screws that can be used in the fixation of soft tissue. Such screws may for example, be used to fix soft tissue grafts to bone during crucial ligament reconstruction surgeries of the knee. Examples of such state of the art screws include the RCI screw (Smith & Nephew, Carlsbad, Calif.) and the Arthrex BIO-INTERFERENCE™ Screw (Arthrex, Naples, Fla.).

Bone plates and bone plating systems are also within the scope of the present invention as medico-technological devices. Biodegradable fixation systems consisting of plates, plates and mesh, and mesh, in varying configurations and length, can be attached to bone for reconstruction. Such uses include the fixation of bones of the craniofacial and midfacial skeleton affected by trauma, fixation of zygomatic fractures, or for reconstruction. The plates may also be contoured by molding. Examples of such state of the art devices include the Howmedica LEIBINGER™ Resorbable Fixation System (Howmedica, Rutherford, N.J.),

Also staples such as biodegradable staples that are used for the fixation of soft tissues are examples of medico-technological devices of the present invention. Such staples can be used, for example, to repair vertical longitudinal full thickness tears (i.e. bucket-handle) of the meniscus. An example of such state of the art devices includes the Absorbable Implantable Staple (United States Surgical Corporation, Norwalk, Conn.). Surgical Mesh is yet another example of medico-technological devices of the present invention. Biodegradable surgical mesh may be used in general surgery. For example, surgical meshes are used in the treatment of hernias where the connective tissue has ruptured or as a sling material to support the repositioning and support of the bladder nect for female urinary incontinence. Such meshes also known as plugs can also be employed as soft tissue implants for reinforcement of soft tissue, for example, in the repair of abdominal aponeuroses and the abdominal wall, fascial and capsular defects, and patellar and achilles tendons, and replacement of infraspinatus thedons and cranial cruciate ligaments. Other uses include the bridging of fascial defects, as a trachea or other organ patch, organ salvage, slings (including an intestinal sling), dural grafting material, wound or burn dressing, and as a hemostatic tamponade. Examples of such state of the art meshes include the Brennen Biosynthetic Surgical Mesh Matrix (Brennan Medical, St. Paul, Minn.),

Similarly, repair patches are examples of medico-technological devices of the present invention. Biodegradable repair patches are often used in general surgery. Patches may be used for pericardial closures, the repair of abdominal and thoracic wall defects, inguinal, paracolostomy, ventral, paraumbilical, scrotal, femoral, and other hernias, urethral slings, muscle flap reinforcement, to reinforce staple lines and long incisions, reconstruction of pelvic floor, repair of rectal and vaginal prolapse, suture and staple bolsters, urinary and bladder repair, pledgets and slings, and other soft tissue repair, reinforcement, and reconstruction. Examples of such state of the art patches include the TISSUEGUARD™ product (Bio-Vascular Inc., St. Paul, Minn., USA). In analogy, cardiovascular patches such as biodegradable cardiovascular patches used for vascular patch grafting, (pulmonary artery augmentation), for intracardiac patching, and for patch closure after endarterectomy are examples within the scope of the present invention.

Examples of similar state of the art (non-degradable) patch materials include Sulzer Vascutek FLUOROPASSIC™ patches and fabrics (Sulzer Carbomedics Inc., Austin Tex., USA)

Sutures such as biodegradable sutures generally used for soft tissue approximation in case of short term wound support are examples of medico-technological devices of the present invention, as are orthopedic pins, including bone filling augmentation material, are used for bone and soft tissue fixation. Such devices are employed for the stabilization of wrist, foot, ankle, hand, elbow, shoulder and knee fractures.

Other examples are a) adhesion barriers (biodegradable) used in general surgery to prevent undesirable adhesions, for example after surgery. Examples of such state of the art devices used for these purposes include the Endopath INTERCEED™ Absorbable Adhesion Barrier (Ethicon, Inc.); b) Stents that are employed in a number of medical applications, normally to prevent reocclusion of a vessel. Examples include cardiovascular and gastroenterology stents. Generally these stents are non-degradable. Ureteric and urethral stents are used to relieve obstruction in a variety of benign, malignant and post-traumatic conditions such as the presence of stones and/or stone fragments, or other ureteral obstructions such as those associated with ureteral structure, carcinoma of abdominal organs, retroperitoneal fibrosis or ureteral trauma, or in association with Extracorporeal Shock Wave Lithotripsy. Examples of state of the art stents include the double pigtail ureteral stent (C.R. Bard, inc., Covington, Ga.), SpiraStent (Urosurge, Coralville, Iowa), and the Cook Urological Ureteral and Urethtral Stent (Cook Urological, Spencer, Ind.).

Preferred devices include sutures, suture fasteners, meniscus repair devices, rivets, tacks, staples, screws (including interference screws), bone plates and bone plating systems, surgical mesh, repair patches, slings, cardiovascular patches, orthopedic pins, heart valves and vascular grafts, adhesion barriers, stents, guided tissue repair/regeneration devices, articular cartilage repair devices, nerve guides, tendon repair devices, atrial septal defect repair devices, pericardial patches, bulking and filling agents, vein valves, bone marrow scaffolds, meniscus regeneration devices, ligament and tendon grafts, ocular cell implants, spinal fusion cages, skin substitutes, dural substitutes, bone graft substitutes, bone dowels, wound dressings, tubings, catheters and hemostats. Particular embodiments of medico-technological device of the present invention are catheters, tubings and guide wires.

The medico-technological device of the present invention may also be a container which is used to store for example bodily fluids, in particular blood samples. Such containers may a variety of shapes, in particular for example in the form of test tubes.

The present invention further relates to a method for collecting blood samples comprising a step of collecting a blood sample, optionally storing the blood sample in a container, where the container is coated with the coating composition comprising the polypeptide of the present invention.

### Preferred Items

Preferred items relating to the present invention are disclosed herein below:
1. A polypeptide comprising or consisting of SEQ ID NO:1 or a polypeptide having at least 70% sequence identity with SEQ ID NO:1, or a polypeptide fragment of any one of SEQ ID NO:1, said polypeptide or fragment thereof being capable of inhibiting the activity of a protease of the blood clotting cascade.
2. A polypeptide variant of SEQ ID NO:1 having at least 70% sequence identity with SEQ ID NO:1, or a polypeptide variant of a fragment of SEQ ID NO:1.
3. The polypeptide according to item 1, wherein the polypeptide has at least 80% sequence identity, such as at least 81% sequence identity, e.g. at least 82% sequence identity, such as at least 83% sequence identity, e.g. at least 84% sequence identity, such as at least 85% sequence identity, e.g. at least 86% sequence identity, such as at least 87% sequence identity, e.g. at least 88% sequence identity, such as at least 89% sequence identity, e.g. at least 90% sequence identity, such as at least 91% sequence identity, e.g. at least 92% sequence identity, such as at least 93% sequence identity, e.g. at least 94% sequence identity, such as at least 95% sequence identity, e.g. at least 96% sequence identity, such as at least 97% sequence identity, e.g. at least 98% sequence identity, such as at least 99% sequence identity, e.g. at least 99.5% sequence identity to SEQ ID NO:1, or a fragment of SEQ ID NO:1.
4. The polypeptide according to item 1, wherein the polypeptide fragment contains less than 99.5%, such as less than 98%, e.g. less than 97%, such as less than 96%, e.g. less than 95%, such as less than 94%, e.g. less than 93%, such as less than 92%, e.g. less than 91%, such as less than 90%, e.g. less than 88%, such as less than 86%, e.g. less than 84%, e.g. less than 82%, such as less than 80%, e.g. less than 75%, such as less than 70%, e.g. less than 65%, such as less than 60%, e.g. less than 55%, such as less than 50%, e.g. less than 45%, such as less than 40%, e.g. less than 35%, such as less than 30%, e.g. less than 25%, such as less than 20%, such as less than 15%, e.g. less than 10% of the amino acid residues of SEQ ID NO:1.
5. The polypeptide according to item 4, wherein the fragment contains less than 220 consecutive amino acid residues of SEQ ID NO:1, such as less than 215 consecutive amino acid residues, e.g. less than 210 consecutive amino acid residues, such as less than 205 consecutive amino acid residues, e.g. less than 200 consecutive amino acid residues, such as less than 195 consecutive amino acid residues, e.g. less than 190 consecutive amino acid residues, such as less than 185 consecutive amino acid residues, e.g. less than 180 consecutive amino acid residues, such as less than 175 consecutive amino acid residues, e.g. less than 170 consecutive amino acid residues, such as less than 165 consecutive amino acid residues, e.g. less than 160 consecutive amino acid residues, such as less than 155 consecutive amino acid residues, e.g. less than 150 consecutive amino acid residues, such as less than 145 consecutive amino acid residues, e.g. less than 140 consecutive amino acid residues, such as less than 135 consecutive amino acid residues, e.g. less than 130 consecutive amino acid residues, such as less than 125 consecutive amino acid residues, e.g. less than 120 consecutive amino acid residues, such as less than 115 consecutive amino acid residues, e.g. less than 110 consecutive amino acid residues, such as less than 105 consecutive amino acid residues, e.g. less than 100 consecutive amino acid residues, such as less than 95 consecutive amino acid residues, e.g. less than 90 consecutive amino acid residues, such as less than 85 consecutive amino acid residues, e.g. less than 80 consecutive amino acid residues, such as less than 75, e.g. less than 60, such as less than 45 consecutive amino acid residues of SEQ ID NO:1.
6. The polypeptide according to item 5, wherein the fragment contains 6 or more consecutive amino acid residues, such as 7 or more consecutive amino acid residues, e.g. 8 or more consecutive amino acid residues, such as 9 or more consecutive amino acid residues, e.g. 10 or more consecutive amino acid residues, such as 12 or more consecutive amino acid residues, e.g. 14 or more consecutive amino acid residues, such as 16 or more consecutive amino acid residues, e.g. 18 or more consecutive amino acid residues, such as 20 or more consecutive amino acid residues, e.g. 22 or more consecutive amino acid residues, such as 24 or more consecutive amino acid residues, e.g. 26 or more consecutive amino acid residues, such as 28 or more consecutive amino acid residues, e.g. 30 or more consecutive amino acid residues of SEQ ID NO:1.
7. The polypeptide variant according to item 2, wherein the polypeptide variant has at least 80% sequence identity, such as at least 81% sequence identity, e.g. at least 82% sequence identity, such as at least 83% sequence identity, e.g. at least 84% sequence identity, such as at least 85% sequence identity, e.g. at least 86% sequence identity, such as at least 87% sequence identity, e.g. at least 88% sequence identity, such as at least 89% sequence identity, e.g. at least 90% sequence identity, such as at least 91% sequence identity, e.g. at least 92% sequence identity, such as at least 93% sequence identity, e.g. at least 94% sequence identity, such as at least 95% sequence identity, e.g. at least 96% sequence identity, such as at least 97% sequence identity, e.g. at least 98% sequence identity, such as at least 99% sequence identity, e.g. at least 99.5% sequence identity to SEQ ID NO:1, or a fragment of SEQ ID NO:1.
8. The polypeptide according to item 2, wherein the polypeptide variant fragment contains less than 99.5%, such as less than 98%, e.g. less than 97%, such as less than 96%, e.g. less than 95%, such as less than 94%, e.g. less than 93%, such as less than 92%, e.g. less than 91%, such as less than 90%, e.g. less than 88%, such as less than 86%, e.g. less than 84%, e.g. less than 82%, such as less than 80%, e.g. less than 75%, such as less than 70%, e.g. less than 65%, such as less than 60%, e.g. less than 55%, such as less than 50%, e.g. less than 45%, such as less than 40%, e.g. less than 35%, such as less than 30%, e.g. less than 25%, such as less than 20%, such as less than 15%, e.g. less than 10% of the amino acid residues of SEQ ID NO:1.
9. The polypeptide according to item 8, wherein the polypeptide variant fragment contains less than 220 consecutive amino acid residues of SEQ ID NO:1, such as less than 215 consecutive amino acid residues, e.g. less than 210 consecutive amino acid residues, such as less than 205 consecutive amino acid residues, e.g. less than 200 consecutive amino acid residues, such as less than 195 consecutive amino acid residues, e.g. less than 190 consecutive amino acid residues, such as less than 185 consecutive amino acid residues, e.g. less than 180 consecutive amino acid residues, such as less than 175 consecutive amino acid residues, e.g. less than 170 consecutive amino acid residues, such as less than 165 consecutive amino acid residues, e.g. less than 160 consecutive amino acid residues, such as less than 155 consecutive amino acid residues, e.g. less than 150 consecutive amino acid residues, such as less than 145 consecutive amino acid residues, e.g. less than 140 consecutive amino acid residues, such as less than 135 consecutive amino acid residues, e.g. less than 130 consecutive amino acid residues, such as less than 125 consecutive amino acid residues, e.g. less than 120 consecutive amino acid residues, such as less than 115 consecutive amino acid residues, e.g. less than 110 consecutive amino acid residues, such as less than 105 consecutive amino acid residues, e.g. less than 100 consecutive amino acid residues, such as less than 95 consecutive amino acid residues, e.g. less than 90 consecutive amino acid residues, such as less than 85 consecutive amino acid residues, e.g. less than 80 consecutive amino acid residues, such as less than 75, e.g. less than 60, such as less than 45 consecutive amino acid residues of SEQ ID NO:1.
10. The polypeptide according to item 9, wherein the polypeptide variant fragment contains 6 or more consecutive amino acid residues, such as 7 or more consecutive amino acid residues, e.g. 8 or more consecutive amino acid residues, such as 9 or more consecutive amino acid residues, e.g. 10 or more consecutive amino acid residues, such as 12 or more consecutive amino acid residues, e.g. 14 or more consecutive amino acid residues, such as 16 or more consecutive amino acid residues, e.g. 18 or more consecutive amino acid residues, such as 20 or more consecutive amino acid residues, e.g. 22 or more consecutive amino acid residues, such as 24 or more consecutive amino acid residues, e.g. 26 or more consecutive amino acid residues, such as 28 or more consecutive amino acid residues, e.g. 30 or more consecutive amino acid residues of SEQ ID NO:1.
11. The polypeptide according to any of items 1 to 10 attached to a carrier.
12. The polypeptide according to item 11 wherein the carrier comprises an avidin moiety, such as streptavidin, which is optionally biotinylated.
13. The polypeptide according to any of items 1 to 10 attached, such as covalently bound, to a solid support or a semi-solid support.
14. The polypeptide according to any of items 1 to 10 operably fused to an affinity tag, such as a His-tag.
15. A fusion polypeptide comprising the polypeptide according to any of items 1 to 10 operably fused to an N-terminal flanking sequence.
16. A fusion polypeptide comprising the polypeptide according to any of items 1 to 10 operably fused to a C-terminal flanking sequence.
17. The polypeptide according to any of items 1 to 10 operably fused to a signal peptide.
18. The polypeptide according to any of items 1 to 10 operably fused to a pro-region.
19. The polypeptide according to any of items 1 to 10 operably fused to a pre-pro-region.
20. The polypeptide according to any of items 1 to 10, wherein one or more amino acid residues are modified, said modification(s) preferably being selected from the group consisting of in vivo or in vitro chemical derivatization, such as acetylation or carboxylation, glycosylation, such as glycosylation resulting from exposing the polypeptide to enzymes which affect glycosylation, for example mammalian glycosylating or deglycosylating enzymes, phosphorylation, such as modification of amino acid residues which results in phosphorylated amino acid residues, for example phosphotyrosine, phosphoserine and phosphothreonine.
21. The polypeptide according to any of items 1 to 10, wherein one or more amino acid residues are modified so as to preferably improve the resistance to proteolytic degradation and stability or to optimize solubility properties or to render the polypeptide more suitable as a therapeutic agent.
22. The polypeptide according to item 21 comprising amino acid residues other than naturally occurring L-amino acid residues.
23. The polypeptide according to item 22 comprising D-amino acid residues.
24. The polypeptide according to item 22 comprising non-naturally occurring, synthetic amino acids.
25. The polypeptide according to any of items 1 to 10 further comprising one or more blocking groups, preferably in the form of chemical substituents suitable to protect and/or stabilize the N- and C-termini of the polypeptide from undesirable degradation.
26. The polypeptide according to item 25, wherein the one or more blocking groups include protecting groups which do not adversely affect in vivo activities of the polypeptide.
27. The polypeptide according to item 25, wherein the one or more blocking groups are introduced by alkylation or acylation of the N-terminus.
28. The polypeptide according to item 25, wherein the one or more blocking groups are selected from N-terminal blocking groups comprising C₁ to C₅ branched or non-branched alkyl groups and acyl groups, such as formyl and acetyl groups, as well as substituted forms thereof, such as the acetamidomethyl (Acm) group.
29. The polypeptide according to item 25, wherein the one or more blocking groups are selected from N-terminal blocking groups comprising desamino analogs of amino acids, which are either coupled to the N-terminus of the peptide or used in place of the N-terminal amino acid residue.
30. The polypeptide according to item 25, wherein the one or more blocking groups are selected from C-terminal blocking groups wherein the carboxyl group of the C-terminus is either incorporated or not, such as esters, ketones, and amides, as well as descarboxylated amino acid analogues.
31. The polypeptide according to item 25, wherein the one or more blocking groups are selected from C-terminal blocking groups comprising ester or ketone-forming alkyl groups, such as lower (C₁ to C₆) alkyl groups, for example methyl, ethyl and propyl, and amide-forming amino groups, such as primary amines (—NH₂), and mono- and di-alkylamino groups, such as methylamino, ethylamino, dimethylamino, diethylamino, methylethylamino, and the like.
32. The polypeptide according to item 25, wherein free amino group(s) at the N-terminal end and free carboxyl group(s) at the termini can be removed altogether from the polypeptide to yield desamino and descarboxylated forms thereof without significantly affecting the biological activity of the polypeptide.
33. An acid addition salt of the polypeptide according to any of items 1 to 32, said salt being obtainable by treating the polypeptide with an inorganic acid, such as hydrochloric acid, hydrobromic acid, sulfuric acid, nitric acid, phosphoric acid, and the like, or an organic acid such as an acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, or salicylic acid, to provide a water soluble salt of the polypeptide.
34. A method for producing the polypeptide according to any of items 1 to 32, said method comprising the steps of collecting potato tuber from Solanum tuberosum and extracting and purifying the polypeptide according to any of items 1 to 32.
35. A method for producing the polypeptide according to any of items 1 to 32, said method comprising the steps of providing a polynucleotide encoding said polypeptide and expressing said polynucleotide either in vitro, or in vivo in a suitable host organism, thereby producing the polypeptide according to any of items 1 to 32.
36. A polynucleotide encoding the polypeptide according to any of items 1 to 32.
37. A nucleotide sequence capable of hybridizing to the polynucleotide encoding the polypeptide according to any of items 1 to 32, or a fragment hereof, under stringent conditions.
38. A nucleotide sequence according to item 37, wherein the portion of said polynucleotide which encodes the polypeptide hybridizes under stringent conditions to a nucleotide probe corresponding to at least 10 consecutive nucleotides of a nucleotide sequence of SEQ ID NO:2.
39. An expression vector comprising the polynucleotide according to item 36, said polynucleotide being optionally operably linked to regulatory sequence controlling the expression of said polynucleotide in a suitable host cell.
40. An isolated recombinant or transgenic host cell comprising the polypeptide according to any of items 1 to 32 and/or the polynucleotide according to item 36 and/or the expression vector according to item 39.
41. A method for generating a recombinant or transgenic host cell, said method comprising the steps of providing a polynucleotide encoding a polypeptide according to any of items 1 to 32, introducing said polynucleotide into said recombinant or transgenic host cell and optionally also expressing said polynucleotide in said recombinant or transgenic host cell, thereby generating a recombinant or transgenic host cell producing said polypeptide.
42. A transgenic, mammalian organism comprising the host cell according to item 40.
43. The transgenic, mammalian organism according to item 42, wherein said mammalian host cell is an animal cell selected from the monophyletic group Bilateria, including a mammalian cell belonging to any of the four major lineages Deuterostomes, Ecdysozoa, Platyzoa and Lophotrochozoa.
44. The transgenic, mammalian organism according to item 42, wherein said mammalian host cell is an animal cell selected from the group consisting of a Blastomere cell, an Egg cell, an Embryonic stem cell, an Erythrocyte, a Fibroblast, a Hepatocyte, a Myoblast, a Myotube, a Neuron, an Oocyte, an Osteoblast, an Osteoclast, a Sperm cell, a T-Cell and a Zygote.
45. A method for generating a transgenic, mammalian host cell, said method comprising the steps of providing a polynucleotide encoding a polypeptide according to any of items 1 to 32, introducing said polynucleotide into said recombinant or transgenic host cell and optionally also expressing said polynucleotide in said transgenic, mammalian host cell, thereby generating a transgenic, mammalian host cell producing said polypeptide.
46. A transgenic plant comprising the host cell according to item 40.
47. The transgenic, plant host cell according to item 46, wherein said host cell is a plant cell of the taxon Embryophyta or Viridiplantae or Chlorobionta, preferably selected from the group consisting of Aleurone cells, Collenchyma cells, Endodermis cells, Endosperm cells, Epidermis cells, Mesophyll cells, Meristematic cells, Palisade cells, Parenchyma cells, Phloem sieve tube cells, Pollen generative cells, Pollen vegetative cells, Sclerenchyma cells, Tracheids cells, Xylem vessel cells and Zygote cells.
48. The transgenic plant according to item 46, wherein said plant is a potato plant.
49. A method for generating a transgenic plant, said method comprising the steps of providing a polynucleotide encoding a polypeptide according to any of items 1 to 32, introducing said polynucleotide into said plant and optionally also expressing said polynucleotide in said plant, thereby generating a transgenic plant producing said polypeptide.
50. A recombinant bacterial host cell comprising the polypeptide according to any of items 1 to 32 and/or the polynucleotide according to item 36 and/or the vector according to item 39.
51. The bacterial host cell according to item 50, wherein said bacterial host cell is selected from a Gram-positive bacterial host cell and a Gram-negative bacterial host cell.
52. A method for generating a recombinant bacterial cell, said method comprising the steps of providing a polynucleotide encoding a polypeptide according to any of items 1 to 32, introducing said polynucleotide into said bacterial cell and optionally also expressing said polynucleotide in said bacterial cell, thereby generating a recombinant bacterial cell producing said polypeptide.
53. A recombinant yeast cell comprising the polypeptide according to any of items 1 to 32 and/or the polynucleotide according to item 36 and/or the vector according to item 39.
54. The yeast host cell according to item 53, wherein said yeast host cell belongs to the genera of Saccharomyces, Scizosacchomyces or Pichia.
55. The yeast host cell according to item 54, wherein said yeast is a Saccharomyces cerevisiae.
56. The yeast host cell according to item 54, wherein said yeast is a Scizosacchomyces pompe.
57. The yeast host cell according to item 54, wherein said yeast is a Pichia pastoris.
58. A method for generating a recombinant yeast cell, said method comprising the steps of providing a polynucleotide encoding a polypeptide according to any of items 1 to 32, introducing said polynucleotide into said yeast cell and optionally also expressing said polynucleotide in said yeast cell, thereby generating a recombinant yeast cell producing said polypeptide.
59. A recombinant fungal host cell comprising the polypeptide according to any of items 1 to 32 and/or the polynucleotide according to item 36 and/or the vector according to item 39.
60. The fungal host cell according to item 59, wherein said fungal cell belongs to the genus of Aspergillus.
61. A method for generating a recombinant fungal cell, said method comprising the steps of providing a polynucleotide encoding a polypeptide according to any of items 1 to 32, introducing said polynucleotide into said fungal cell and optionally also expressing said polynucleotide in said fungal cell, thereby generating a recombinant bacterial cell producing said polypeptide.
62. An antibody, or a binding fragment thereof, specific for the polypeptide or a fragment thereof according to any of items 1 to 32.
63. The antibody according to item 62, wherein said antibody is polyclonal.
64. The antibody according to item 62, wherein said antibody is monoclonal.
65. The antibody fragment according to item 62, wherein said antibody fragment comprises a portion of an antibody selected from the group consisting of F(ab′)₂, F(ab)₂, Fab′ and Fab.
66. The antibody fragment according to item 62, wherein said antibody fragment is synthetic or a genetically engineered polypeptide that binds to a specific antigen.
67. The antibody fragment according to item 62, wherein said antibody fragment is selected from the group consisting of antibody fragments comprising or consisting of the light chain variable region, antibody fragments comprising or consisting of a “Fv” fragment consisting of the variable regions of the heavy and light chains, antibody fragments comprising or consisting of recombinant single chain polypeptide molecules in which light and heavy variable regions are connected by a peptide linker (“scFv proteins”) and antibody fragments comprising or consisting of minimal recognition units consisting of the amino acid residues that mimic the hypervariable region.
68. The antibody according to item 62, wherein said antibody is a chimeric antibody in the form of a recombinant protein that contains the variable domains and complementary determining regions derived from a rodent antibody, while the remainder of the antibody molecule is derived from a human antibody.
69. The antibody according to item 62, wherein said antibody is a humanized antibody in the form of a recombinant protein in which murine complementarity determining regions of a monoclonal antibody have been transferred from heavy and light variable chains of the murine immunoglobulin into a human variable domain.
70. The antibody according to any of items 62 to 69 further comprising or being associated with a detectable label in the form of a molecule or atom which can be conjugated to an antibody moiety to produce a moiety which can be more easily detected.
71. The antibody according to item 69, wherein the label is selected from the group consisting of chelators, photoactive agents, radioisotopes, fluorescent agents and paramagnetic ions.
72. A method for generating a polyclonal antibody, or a binding fragment thereof specific for the polypeptide according to any of items 1 to 32, said method comprising the steps of immunizing a mammalian subject with the polypeptide according to any of items 1 to 32 under conditions eliciting an antibody response, identifying an antibody which bind specifically to the polypeptide, and optionally isolating said antibody or binding fragment thereof from said mammalian subject.
73. A method for generating a monoclonal antibody specific for the polypeptide according to any of items 1 to 32, said method comprising the steps of immunizing a mammalian subject with the polypeptide according to any of items 1 to 32 under conditions eliciting an antibody response, preparing a hybridoma producing a monoclonal antibody specific for the polypeptide according to any of items 1 to 32, and identifying an antibody which bind specifically to the polypeptide.
74. A polypeptide capable of being recognized by the antibody according to any of items 62-72.
75. A composition comprising the polypeptide according to any of items 1 to 32 in combination with a physiologically acceptable carrier.
76. A pharmaceutical composition comprising the polypeptide according to any of items 1 to 32 in combination with a pharmaceutically acceptable carrier.
77. A composition according to items 75 and 76 further comprising the combination with one or more additional bioactive agent(s) acting on platelet aggregation (anti-platelet agent) in hemostasis for medical use.
78. The composition according to item 77 wherein the one or more bioactive agent is selected from the group consisting of: aspirin, aloxiprin, ditazole, carbasalate calcium, cloricromen, indobufen, picotamide, triflusal, clopidogrel, dipyridamole, prasugrel, ticlopidine, beraprost, prostacyclin, iloprost, treprostini, abciximab, eptifibatide and tirofiban.

79. A composition according to items 75 and 76 further comprising the combination with one or more additional bioactive agent(s) acting on the blood coagulation cascade (anti-coagulant agent) in hemostasis for medical use.
80. The composition according to item 79 wherein the one or more bioactive agent(s) is selected from the group consisting of: heparin, low-molecular weight heparin, bemiparin, dalteparin, enoxaparin, nadroparin, parnaparin, reviparin, tinzaparin, sulodexide, danaparoid, warfarin, acenocoumarol, clorindione, coumatetralyl, dicumarol, diphenadione, ethyl biscoumacetate, phenprocoumon, phenindione, tioclomarol, dabigatran, idraparinux, lepirudin, bivalirudin, argatroban, desirudin, hirudin, melagatran, ximelagatran, antithrombin III, rivaroxaban, fondaparinux, protein C, protein S, tissue-factor pathway inhibitor, defibrotide and dermatan sulphate.
81. A composition according to items 75 and 76 further comprising the combination with one or more additional bioactive agent(s) acting on fibrinolysis (fibrinolytic agent) in hemostasis for medical use.
82. The composition according to item 81 wherein the one or more bioactive agent(s) is selected from the group consisting of tenecteplase, anistreplase, ancrod, drotrecogin, fibrinolysin, brinase, tissue pasminogen activator, urokinase-type plasminogen activator, urokinase and streptokinase.
83. A composition according to items 75 and 76 further comprising the combination with one or more additional bioactive agent(s) selected from the group of anti-platelet, anti-coagulation and fibrinolytic agent(s) for medical use.
84. Kit-of-parts comprising the polypeptide according to item 1-32 or the composition according to items 75-76, and at least an additional component.
85. The kit-of-parts according to item 84, wherein said additional one or more component(s) comprise an instruction pamphlet of desirable administration and dosis regiment.
86. The kit-of-parts according to item 84, wherein at least one or more said additional component(s) are bioactive agent(s) selected from the group cited in items 78, 81 and 84.
87. A method for identifying binding partners for the polypeptide according to item 1-32, said method comprising the steps of extracting the polypeptide and isolating said binding partners.
88. The method according to item 87, wherein said binding partner comprise one or more agonists or antagonists.
89. A polypeptide according to any of items 1 to 32 or the composition according to items 75-76 for use as a medicament.
90. A method for treatment of an individual in need thereof with the agonists or antagonists according to item 88.
91. A method for treatment of an individual in need thereof with the agonists or antagonists according to item 88 in combination with the polypeptide according to items 1-32 or the composition according to items 75-76.
92. A method for treatment of coronary syndromes comprising administration of the composition according to item 75-76 to an individual in need thereof.
93. The method according to item 92, wherein said coronary syndrome is due to occlusion of coronary arteries and comprises stable angina pectoris, unstable angina pectoris, myocardial ischemia and myocardial infarction.
94. The method according to item 92, wherein said coronary syndrome comprises dilated cardiomyopathy, hypertropic cardiomyopathy, congestive heart failure and cardiac failure.
95. The method according to item 92, wherein said treatment is prophylactic, said prophylactic treatment being either primary or secondary.
96. The method according to item 92, wherein said treatment is ameliorating.
97. The method according to item 92, wherein said treatment is curative.
98. The use of the composition according to items 75-76 for the manufacture of a medicament for therapeutic applications targeted at the coronary syndromes selected from the group consisting of: stable angina pectoris, unstable angina pectoris, myocardial ischemia, myocardial infarction, dilated cardiomyopathy, hypertropic cardiomyopathy, congestive heart failure and cardiac failure.
99. The composition according to items 75-76 for treatment of coronary syndromes selected from the group consisting of: stable angina pectoris, unstable angina pectoris, myocardial ischemia, myocardial infarction, dilated cardiomyopathy, hypertropic cardiomyopathy, congestive heart failure and cardiac failure.
100. A pharmaceutical composition for treating coronary syndromes selected from the group consisting of: stable angina pectoris, unstable angina pectoris, myocardial ischemia, myocardial infarction, dilated cardiomyopathy, hypertropic cardiomyopathy, congestive heart failure and cardiac failure, comprising the composition according to items 75-76.
101. A method for treatment of atrial fibrillation, comprising administration of the composition according to item 75-76 to an individual in need thereof.
102. The method according to item 101, wherein said atrial fibrillation is treated by cardioversion.
103. The method according to item 101, wherein said treatment is prophylactic, said prophylactic treatment being primary or secondary.
104. The use of the composition according to items 75-76 for the manufacture of a medicament for therapeutic applications of atrial fibrillation and cardioversion.
105. The composition according to items 75-76 for treatment of atrial fibrillation and cardioversion.
106. A pharmaceutical composition for treating atrial fibrillation and cardioversion comprising the composition according to items 75-76.
107. A method for treatment of peripheral arterial occlusion comprising administration of the composition according to item 75-76 to an individual in need thereof.
108. The method according to item 107, wherein said peripheral arterial occlusion is caused by primary or recurrent thrombus formation, embolism or atherosclerosis.
109. The method according to item 107, wherein said treatment is prophylactic, said prophylactic treatment being primary or secondary.
110. The method according to item 107, wherein said treatment is ameliorating.
111. The method according to item 107, wherein said treatment is curative.
112. The use of the composition according to items 75-76 for the manufacture of a medicament for therapeutic applications targeted at peripheral arterial occlusion caused by primary or recurrent thrombus formation, embolism or atherosclerosis.
113. The composition according to items 75-76 for treatment of peripheral arterial occlusion caused by primary or recurrent thrombus formation, embolism or atherosclerosis.
114. A pharmaceutical composition for treating peripheral arterial occlusion caused by primary or recurrent thrombus formation, embolism or atherosclerosis, comprising the composition according to items 75-76.
115. A method for treatment of deep-vein thrombosis comprising administration of the composition according to item 75-76 to an individual in need thereof.
116. The method according to item 115, wherein said treatment prevents or cures pulmonary embolism.
117. The method according to item 115, wherein said treatment is prophylactic, said prophylactic treatment being primary or secondary.
118. The method according to item 115, wherein said treatment is ameliorating.
119. The method according to item 115, wherein said treatment is curative.
120. The use of the composition according to items 75-76 for the manufacture of a medicament for therapeutic applications targeted at deep-vein thrombosis and pulmonary embolism.
121. The composition according to items 75-76 for treatment of deep-vein thrombosis and pulmonary embolism.
122. A pharmaceutical composition for treating deep-vein thrombosis and pulmonary embolism comprising the composition according to items 75-76.
123. A method for treatment of blood clotting in extracorporal circuits and catheters, comprising administration of the composition according to item 75-76 to an individual in need thereof.
124. The method according to item 123, wherein said extracorporal circuit encompasses cardiopulmonary bypass circuits.
125. The method according to item 123, wherein said extracorporal circuit encompasses hemodialysis bypass circuits.
126. The method according to item 123, wherein said extracorporal catheter encompasses a central line catheter (CVK line).
127. The method according to item 123, wherein said treatment is prophylactic.
128. The use of the composition according to items 75-76 for the manufacture of a medicament for therapeutic applications targeted against blood clotting in extracorporal circuits and catheters during cardiopulmonary bypass, hemodialysis and CVK.
129. The composition according to items 75-76 for treatment of blood clotting in extracorporal circuits during cardiopulmonary bypass, hemodialysis and CVK.
130. A pharmaceutical composition for treating blood clotting in extracorporal circuits during cardiopulmonary bypass and hemodialysis comprising the composition according to items 75-76.
131. A method for treatment of blood clotting during angioplastic procedures, comprising administration of the composition according to item 75-76 to an individual in need thereof.
132. The method according to item 131, wherein said angioplastic procedures comprise coronary angioplasty, peripheral angioplasty, renal angioplasty and carotid angioplasty.
133. The method according to items 131, wherein said treatment is prophylactic.
134. The use of the composition according to items 75-76 for the manufacture of a medicament for therapeutic applications targeted against blood clotting during angioplastic procedures.
135. The composition according to items 75-76 for treatment of blood clotting during angioplastic procedures.
136. A pharmaceutical composition for treating blood clotting during angioplastic procedures comprising the composition according to items 75-76.
137. A method for treatment of blood clotting in connection with artificial heart valve replacement, comprising administration of the composition according to item 75-76 to an individual in need thereof.
138. The method according to item 137, said artificial heart valve replacement being mechanical or bioprosthetic.
139. The method according to item 137, wherein said treatment is prophylactic.
140. The use of the composition according to items 75-76 for the manufacture of a medicament for therapeutic applications targeted against blood clotting in connection with prosthetic heart valve replacement.
141. The composition according to items 75-76 for treatment of blood clotting in connection with prosthetic heart valve replacement.
142. A pharmaceutical composition for treating blood clotting in connection with prosthetic heart valve replacement comprising the composition according to items 75-76.
143. A method for treatment of blood clotting due to diseases affecting the heart valves, comprising administration of the composition according to item 75-76 to an individual in need thereof.
144. The method according to item 143, wherein said heart valve disease comprising infected valves (bacterial endocarditis), rheumatic mitral valve disease, mitral stenosis, mitral valve prolapse, mitral annular calcification and isolated aortic valve disease.
145. The method according to item 143, wherein said treatment is prophylactic, said prophylactic treatment being primary or secondary.
146. The use of the composition according to items 75-76 for the manufacture of a medicament for therapeutic applications targeted against blood clotting due to diseases affecting the heart valves comprising infected valves (bacterial endocarditis), rheumatic mitral valve disease, mitral stenosis, mitral valve prolapse, mitral annular calcification and isolated aortic valve disease.
147. The composition according to items 75-76 for treatment of blood clotting due to diseases affecting the heart valves comprising infected valves (bacterial endocarditis), rheumatic mitral valve disease, mitral stenosis, mitral valve prolapse, mitral annular calcification and isolated aortic valve disease.
148. A pharmaceutical composition for treating blood clotting due to diseases affecting the heart valves comprising infected valves (bacterial endocarditis), rheumatic mitral valve disease, mitral stenosis, mitral valve prolapse, mitral annular calcification and isolated aortic valve disease comprising the composition according to items 75-76.
149. A method for treatment of blood clotting in patients with thrombophilia syndromes, comprising administration of the composition according to item 75-76 to an individual in need thereof.
150. The method according to item 149, wherein the thrombophilia syndrome comprises the following causes: antiphospholipid syndrome, Factor V Leiden, prothrombin mutation/factor II mutation, high homocysteine levels due to MTHFR mutation or vitamin deficiency (vitamins B6, B12 and folic acid), renal loss of antithrombin, plasminogen and fibrinolysis disorders, paroxysmal nocturnal hemoglobinuria, protein C deficiency, protein S deficiency, and antithrombin III deficiency.
151. The method according to item 149, wherein said treatment is prophylactic.
152. The method according to item 149, wherein said treatment is ameliorating.
153. The method according to item 149, wherein said treatment is curative.
154. The use of the composition according to items 75-76 for the manufacture of a medicament for therapeutic applications targeted against blood clotting in patients with thrombophilia syndromes including antiphospholipid syndrome, Factor V Leiden, prothrombin mutation/factor II mutation, high homocysteine levels due to MTHFR mutation or vitamin deficiency (vitamins B6, B12 and folic acid), renal loss of antithrombin, plasminogen and fibrinolysis disorders, paroxysmal nocturnal hemoglobinuria, protein C deficiency, protein S deficiency, and antithrombin III deficiency.
155. The composition according to items 75-76 for treatment of blood clotting in patients with thrombophilia syndromes including antiphospholipid syndrome, Factor V Leiden, prothrombin mutation/factor II mutation, high homocysteine levels due to MTHFR mutation or vitamin deficiency (vitamins B6, B12 and folic acid), renal loss of antithrombin, plasminogen and fibrinolysis disorders, paroxysmal nocturnal hemoglobinuria, protein C deficiency, protein S deficiency, and antithrombin III deficiency.
156. A pharmaceutical composition for treating blood clotting in patients with thrombophilia syndromes including antiphospholipid syndrome, Factor V Leiden, prothrombin mutation/factor II mutation, high homocysteine levels due to MTHFR mutation or vitamin deficiency (vitamins B6, B12 and folic acid), renal loss of antithrombin, plasminogen and fibrinolysis disorders, paroxysmal nocturnal hemoglobinuria, protein C deficiency, protein S deficiency, and antithrombin III deficiency comprising the composition according to items 75-76.
157. A method for treatment of blood clotting following coagulation management, comprising administration of the composition according to item 75-76 to an individual in need thereof.
158. The method according to item 157, wherein said coagulation management is warranted by the presence of conditions that increase the risk of bleeding comprising Hemophilia A, Hemophilia B, Hemophilia C, Von Willebrand disease, major blood loss, Glanzmann's thrombasthenia, Bernard-Soulier syndrome, gray platelet syndrome and delta storage pool deficiency.
159. The method according to item 157, said treatment being ameliorating.
160. The method according to item 157, said treatment being curative.
161. The use of the composition according to items 75-76 for the manufacture of a medicament for therapeutic applications targeted against blood clotting following coagulation management warranted by the presence of conditions that increase the risk of bleeding comprising Hemophilia A, Hemophilia B, Hemophilia C, Von Willebrand disease, major blood loss, Glanzmann's thrombasthenia, Bernard-Soulier syndrome, gray platelet syndrome and delta storage pool deficiency.
162. The composition according to items 75-76 for treatment of blood clotting following coagulation management warranted by the presence of conditions that increase the risk of bleeding comprising Hemophilia A, Hemophilia B, Hemophilia C, Von Willebrand disease, major blood loss, Glanzmann's thrombasthenia, Bernard-Soulier syndrome, gray platelet syndrome and delta storage pool deficiency.
163. A pharmaceutical composition for treating blood clotting following coagulation management warranted by the presence of conditions that increase the risk of bleeding comprising Hemophilia A, Hemophilia B, Hemophilia C, Von Willebrand disease, major blood loss, Glanzmann's thrombasthenia, Bernard-Soulier syndrome, gray platelet syndrome and delta storage pool deficiency, comprising the composition according to items 75-76.
164. A method for treatment of decreased platelet numbers leading to increased platelet activation comprising administration of the composition according to item 75-76 to an individual in need thereof.
165. The method according to item 164, wherein the decreased platelet number is caused by insufficient production (e.g. in myelodysplastic syndrome or other bone marrow disorders), destruction by the immune system (immune thrombocytopenic purpura/ITP), and consumption due to various causes (thrombotic thrombocytopenic purpura/TTP, hemolytic-uremic syndrome/HUS, paroxysmal nocturnal hemoglobinuria/PNH, disseminated intravascular coagulation/DIC, heparin-induced thrombocytopenia/HIT).
166. The method according to item 164, wherein said treatment is prophylactic, said prophylactic treatment being primary or secondary.
167. The use of the composition according to items 75-76 for the manufacture of a medicament for therapeutic applications targeted against blood clotting due to decreased platelet number caused by insufficient production (e.g. in myelodysplastic syndrome or other bone marrow disorders), destruction by the immune system (immune thrombocytopenic purpura/ITP), and consumption due to various causes (thrombotic thrombocytopenic purpura/TTP, hemolytic-uremic syndrome/HUS, paroxysmal nocturnal hemoglobinuria/PNH, disseminated intravascular coagulation/DIC, heparin-induced thrombocytopenia/HIT).
168. The composition according to items 75-76 for treatment of blood clotting due to decreased platelet number caused by insufficient production (e.g. in myelodysplastic syndrome or other bone marrow disorders), destruction by the immune system (immune thrombocytopenic purpura/ITP), and consumption due to various causes (thrombotic thrombocytopenic purpura/TTP, hemolytic-uremic syndrome/HUS, paroxysmal nocturnal hemoglobinuria/PNH, disseminated intravascular coagulation/DIC, heparin-induced thrombocytopenia/HIT).
169. A pharmaceutical composition for treating blood clotting due to decreased platelet number caused by insufficient production (e.g. in myelodysplastic syndrome or other bone marrow disorders), destruction by the immune system (immune thrombocytopenic purpura/ITP), and consumption due to various causes (thrombotic thrombocytopenic purpura/TTP, hemolytic-uremic syndrome/HUS, paroxysmal nocturnal hemoglobinuria/PNH, disseminated intravascular coagulation/DIC, heparin-induced thrombocytopenia/HIT), comprising the composition according to items 75-76.
170. A method for treatment of blood clotting for any reason in individuals who do not tolerate other medicaments targeting blood clotting on the market, comprising administration of the composition according to item 75-76 to an individual in need thereof.
171. The method according to item 170, said treatment being prophylactic, said prophylactic treatment being primary or secondary.
172. The method according to item 170, said treatment being ameliorating.
173. The method according to item 170, said treatment being curative.
174. The use of the composition according to items 75-76 for the manufacture of a medicament for therapeutic applications against blood clotting for any reason in individuals who do not tolerate other medicaments targeting blood clotting on the market.
175. The composition according to items 75-76 for treatment of blood clotting for any reason in individuals who do not tolerate other medicaments targeting blood clotting on the market.
176. A pharmaceutical composition for treating blood clotting in individuals who do not tolerate other medicaments targeting blood clotting on the market comprising the composition according to items 75-76.
177. A method for treatment of blood clotting in individuals that are immobilized for any reason, patients that suffer from critical limb ischemia or have had a limb amputated, comprising administration of the composition according to item 75-76 to an individual in need thereof.
178. The method according to item 177, said treatment being prophylactic.
179. The method according to item 177, said treatment being ameliorating.
180. The use of the composition according to items 75-76 for the manufacture of a medicament for therapeutic applications against blood clotting in individuals that are immobilized for any reason, patients that suffer from critical limb ischemia or have had a limb amputated.
181. The composition according to items 75-76 for treatment of blood clotting in individuals that are immobilized for any reason, patients that suffer from critical limb ischemia or have had a limb amputated.
182. A pharmaceutical composition for treating blood clotting in individuals that are immobilized for any reason, patients that suffer from critical limb ischemia or have had a limb amputated, comprising the composition according to items 75-76.
183. A method for treatment of blood clotting in patients receiving any form for chemotherapeutics, comprising administration of the composition according to item 75-76 to an individual in need thereof.
184. The method according to item 183, said treatment being prophylactic.
185. The use of the composition according to items 75-76 for the manufacture of a medicament for therapeutic applications against blood clotting in patients receiving any form for chemotherapeutics.
186. The composition according to items 75-76 for treatment of blood clotting in patients receiving any form for chemotherapeutics.
187. A pharmaceutical composition for treating blood clotting in patients receiving any form for chemotherapeutics, comprising the composition according to items 75-76.
188. A method for treatment of blood clotting in patients at high risk for developing blood clots, comprising administration of the composition according to item 75-76 to an individual in need thereof.
189. The method according to item 188, said high risk individuals having a history of venous or arterial thrombus formation; for example having coronary syndromes such as stable angina pectoris, unstable angina pectoris, myocardial ischemia, myocardial infarction, atrial fibrillation, cardioversion, dilated cardiomyopathy, hypertropic cardiomyopathy, congestive heart failure or cardiac failure; such individuals with artificial heart valves or diseases of the heart valves; or such individuals with diabetes mellitus, hyperlipidemia, hypertension or atherosclerosis; patients undergoing surgery; cancer patients; cancer patients receiving chemotherapy for neoplastic indications such as breast cancer; immobile or obese individuals; individuals with limb ischemia or amputated limbs; aircraft passengers with economy class syndrome; individuals with hypercoagulative/thrombophilia syndromes such as Factor V Leiden, antiphospholipid syndrome, prothrombin mutation, high homocycteine levels, renal loss of antithrombin e.g. in proteinuria (excessive protein loss from the kidneys), plasminogen and fibrinolysis disorders, paroxysmal nocturnal hemoglobinuria, protein C deficiency, protein S deficiency and antithrombin III deficiency; individuals with decreased platelet numbers caused by myelodysplastic syndrome, bone marrow disorders, immune thrombocytopenic purpora, thrombotic thrombocytopenic purpora, hemolytic-uremic syndrome, disseminated intravascular coagulation and heparin-induced thrombocytopenia;
190. The method according to item 188, said treatment being prophylactic, said prophylactic treatment being primary or secondary.
191. The use of the composition according to items 75-76 for the manufacture of a medicament for therapeutic applications against blood clotting in patients at high risk for developing blood clots.
192. The composition according to items 75-76 for treatment of blood clotting in patients at high risk for developing blood clots.
193. A pharmaceutical composition for treating blood clotting in patients at high risk for developing blood clots, comprising the composition according to items 75-76.

### EXAMPLES

### Example 1

### Purification of a Protein with SEQ ID NO:1 from Potato

### Preparation of Potato Juice

2.5 kg of potato tubers (cv. Kuras) collected in Northern Jutland in 2006 was washed and cut into 3-4 cm pieces. The juice was made with a juice extractor into a beaker containing 10 g of sodium bisulphite and left on ice for 15 min. The supernatant was centrifuged for 20 min at 10000×g at 4° C. The supernatant was filtered (0.45 μm filter) and pH was adjusted to 7-8 with NaOH.

### Precipitation of Potato Juice Proteins

To separate the KPI (Kunitz protease inhibitor) in the potato juice from the other proteins the juice was first exposed to AMS precipitation and secondly to ethanol precipitation.

With Ammonium Sulphate (AMS)

The desired saturation concentration for the precipitation was 70%. 472.28 g AMS pr. liter potato juice was slowly added under stirring on ice. Stirring was continued for 30 min. The solution was centrifuged for 30 min at 10000×g and 4° C. Pellet was resuspended in 275 mL (½ the vol. of potato juice after filtration) 10 mM tris buffer pH 8 in water bath sonication.

With Ethanol

40% cold (−20° C.) ethanol was added under stirring to the potato protein solution. The solution was incubated at 4° C. for an hour and subsequently centrifuged for 10 min at 5000×g at 4° C. The supernatant was dialysed (cut off 6-8 kDa) against 5 L 10 mM tris buffer pH 8 over night and afterwards in fresh buffer for two hours.

### Ion Exchange Chromatography

The protein sample was centrifuged at 10000×g for 20 min. The supernatant was filtered through a glass fibre filter (1.6 μm) and afterwards through a 0.45 μm filter. The protein sample was applied to a Q sepharose HP column, which had been equilibrated with 10 mM tris, pH 8 and the proteins were eluted with a linear gradient of 0-1 M NaCl in 10 mM tris, pH 8 with a flow of 5 mL/min. The flow through was applied to an S sepharose column, equilibrated with 10 mM tris, pH 8. Ion exchange fractions with ability to inhibit plasma coagulation (see example 2) were concentrated 10 times using ultra filtration membranes with a 10 kDa cut off.

### Hydrophobic Interaction Chromatography

The protein sample was subsequently applied to a phenyl sepharose column which had been equilibrated with 10 mM tris, 2 M NaCl, pH 8. The proteins were eluted with a linear gradient of 2-0 M NaCl in 10 mM tris, pH 8 with a flow of 4 mL/min. Fractions were concentrated 10 times using amicon ultra centrifugal filter devices and analyzed on a 15% SDS-gel.

### MS/MS Analyses

Selected protein bands from the SDS-gel were cut out and digested with trypsin. Automated LC-ESI MS/MS was performed using a hybrid QTOF mass spectrometer and an Ultimate nano-HPLC system. Protein homologs were identified using an in-house Mascot database search engine. Analysis of intact proteins was performed by off-line nESI on a hybrid QTOF mass spectrometer using nanospray emitters.

KPI A-k1 (corresponding to SEQ ID1) was present in all the tested fractions. It was concluded that KPI A-K1 most likely is the protein responsible for the inhibit plasma coagulation (see example 2).

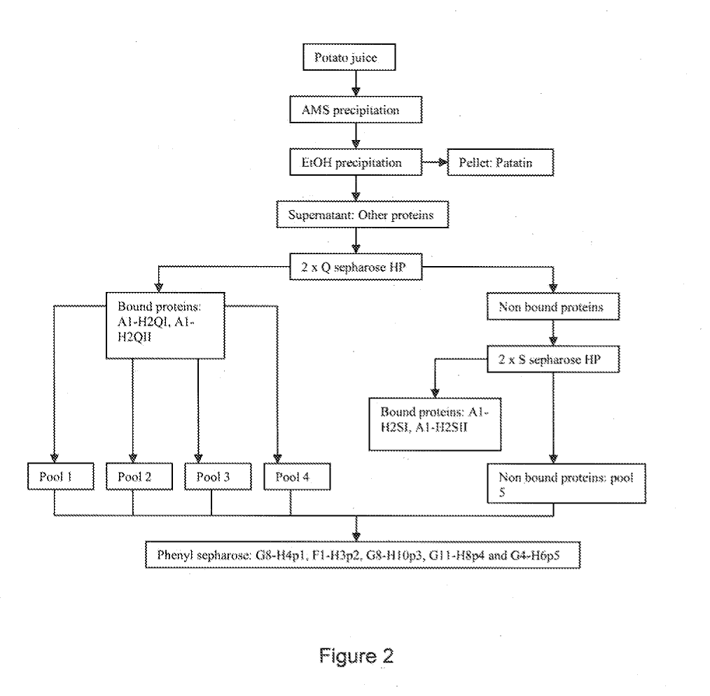

The purification precedure is illustrated in FIG. 2.

### Example 2

### Function of Protein with SEQ ID NO:1

Fractions comprising the protein with SEQ ID1 were purified from potato as described in example 1.

The ability of the fractions to inhibit plasma coagulation was analysed by the inhibitor screening assay described below.

### Separation of Blood

Two part porcine blood (from Danish Crown), and one part 25 mM hepes buffer (25 mM, 0.138 M NaCl, 2.7 mM KCl, pH 7.4) was centrifuged for 25 min at 2400 rpm, 20° C. The plasma was centrifuged again at 9600 rpm (80% of the centrifuge top speed) for 20 min. Finally, the plasma was filtered through a 0.45 μm membrane and stored at −20° C. Human plasma was kindly provided by Clinical Immunological Department at Aalborg Hospital and stored at −80° C.

### Inhibitor Screening Assay

100 μL of plasma was mixed with 30 μL of 25 mM hepes buffer (25 mM, 0.138 M NaCl, 2.7 mM KCl, pH 7.4) and 20 μL of protein sample. 100 μL plasma, 30 μL 25 mM Hepes buffer and 20 μL 0.5 M EDTA or 20 μL buffer, respectively, were used as references. 50 μL of a 0.05 M CaCl₂ solution was added to start the assay, and the samples were flipped and left at room temperature for an hour. If the plasma had coagulated, the added protein solution did not have inhibitor activity, if the plasma had not coagulated, the protein sample was positive for inhibitor activity. The assay was carried out in triplicates.

### Coagulation Inhibitor Assay

Selected fractions from the hydrophobic interaction chromatography and cationic exchange chromatography (example 1) were tested on their ability to inhibit the coagulation of porcine and human plasma at various concentrations. 100 μL plasma, x μL of fraction (the volume was dependent on the desired concentration and the concentration of protein in the fraction sample) and 25 mM Hepes buffer, pH 7.4 up to 150 μL for porcine plasma and 168 μL for human plasma. 0.05 M CaCl₂ up to 200 μL were added to start the assay and the turbidity was measured at 590 nm every 2 min for up to three hours. The assay was carried out in 96 well plates and every concentration was done in triplicates. The coagulation time (c½) was calculated using the following equation:

$$c 1 / 2 = f ( x ) = \frac{{y}_{max} - {y}_{min}}{2} + {y}_{min} \qquad ( 2.2 )$$

### Amidolytic Activity Assay

The different fractions ability to inhibit fXa, thrombin and fVIIa were analyzed using an amidolytic activity assay.

All amidolytic activity assays were carried out in triplicates in 50 mM tris, 100 mM NaCl, 10 mM CaCl₂, 0.1% PEG 6000, pH 8 at 25° C. with chromozym t-PA as substrate.

fVIIa, Thrombin and fXa Inhibition

Amidolytic activity was measured for the three enzymes in the presence of selected inhibitor fractions. The optimal enzyme concentrations had been determined to 25 nM for fXa and 100 nM for fVIIa and thrombin by another amidolytic activity assay (data not shown). The chromozym t-PA concentration was 0.5 mM. Buffer, enzyme and inhibitor were added to a 96 well plate and incubated for 10 min at room temperature. The reactions were started by addition of chromozym t-PA and the change in absorption was measured at 405 nm every 30 sec in an ELISA reader. Absorbances were converted into p-nitroaniline formation (mM) using a molar absorption coefficient of 9900 M⁻¹ cm⁻¹ and a light path length of 0.285 cm. The light path length was calculated from the volume of sample (100 μL), the total volume of the well (382 μL) and the height of the well (10.9 mm).

### Determination of Kinetic Constants

fXa hydrolysis of chromozym t-PA was followed in substrate concentration varying from 0-1.25 mM in the presence of 50 nM enzyme and with or without 9.5 nM a inhibitor fraction and 1 U/mL heparin. Enzyme, inhibitor and heparin were incubated for 10 min before the substrate was added, the absorbance was measured every 30 sec at 405 nm. K_{M} and Vₘₐₓ were obtained by fitting the data to a Michaelis Menten curve using GraphPad Prism version 5.00. K was determined by fitting the data to a competitive inhibition in the same program using the model:

$$Y = \frac{{V}_{max} * x}{{K}_{MObs} + x} where {K}_{MObs} = {K}_{M} * ( 1 + \frac{[ I ]}{{K}_{i}} ) \qquad ( 2.3 )$$

Y is the measured velocity, x is the substrate concentration in mM and I is the inhibitor concentration in mM.

### Results

The fractions analyzed using MS/MS were tested for their ability to inhibit both porcine and human plasma at different concentrations.

All the tested fractions had an effect on the coagulation, some more than others. The results are summarized in table 3 below.

TABLE 3

| | Porcine plasma | | Human plasma | |
|---|---|---|---|---|
| | C_{1/2} ± SD | [I] at 2 · c_{1/2} | c_{1/2} ± SD | [I] at app 2 · c_{1/2} |
| Inhibitor | [min] | [μg/mL] | [min] | [μg/mL] |
| H10p1 | 22 ± 1 | — | 59 ± 3 | 19 |
| H11p1 | 20 ± 1 | 80 | 60 ± 3 | —* |
| H7p2 | 21 ± 0 | — | 48 ± 2 | 11 |
| H9p2 | 19 ± 0 | 70 | 44 ± 1 | 15 |
| H12p2 | 19 ± 1 | 70 | 53 ± 2 | 19 |
| G12p3 | 17 ± 0 | 30 | 67 ± 7 | — |
| H12p3 | 18 ± 1 | 60 | 54 ± 3 | —* |
| G11p4 | 19 ± 1 | 23 | 44 ± 2 | 9 |
| H12p4 | 21 ± 1 | 100 | 45 ± 2 | — |
| G7p5 | 19 ± 0 | 32 | 53 ± 5 | — |
| H7p5 | 19 ± 0 | — | 52 ± 4 | 10 |
| H11p5 | 21 ± 0 | — | 43 ± 3 | 8 |
| A10SI | 21 ± 0 | 15 | 75 ± 0 | —* |
| A11SI | 22 ± 1 | 10 | 80 ± 2 | —* |
| B11SI | 21 ± 1 | 30 | 83 ± 3 | —* |
| A7SII | 22 ± 2 | 40 | 70 ± 2 | — |

Coagulation time (c½) for porcine and human plasma without fraction added, and the inhibitor concentration needed to increase the coagulation time two fold. —indicates that none of the tested concentrations were able to double coagulation time and—indicates that the fraction inhibited the coagulation to such an extent that the concentration that doubled the coagulation time could not be determined with the selected concentrations.

### Determination of the Inhibitor Activity

The fractions from the different pools that showed the best ability to inhibit the plasma coagulation were tested to see if they had any effect on the enzymes fVIIa, fXa, and thrombin from the coagulation cascade.

None of the selected inhibitor fractions showed ability to inhibit fVIIa. Some of the inhibitor fractions showed some inhibition of thrombin. All the tested inhibitor fractions showed some inhibitor effect towards fXa. B11SI has the best inhibitor effect towards fXa.

The kinetic values were calculated and are summarized in table 4 below.

TABLE 4

| | | | fXa with B11SI |
|---|---|---|---|
| | fXa | fXa with B11SI | and heparin |
| Vₘₐₓ [mM * min⁻¹] | 8.8 · 10⁻² ± 4.4 · 10⁻³ | 2.6 · 10⁻² ± 3.3 · 10⁻³ | 2.3 · 10⁻² ± 1.8 · 10⁻³ |
| K_{M} [mM] | 0.23 ± 3.4 · 10⁻² | 1.25 ± 0.27 | 1.18 ± 0.16 |
| Kᵢ [nM] | — | 114.2 ± 16.0 | 101.4 ± 15.1 |
| 95% confidence | | | |
| intervals | | | |
| Vₘₐₓ [mM * min⁻¹] | [7.8 · 10⁻²; 9.8 · 10⁻²] | [1.8 · 10⁻²; 3.4 · 10⁻²] | [1.8 · 10⁻²; 2.7 · 10⁻²] |
| K_{M} [mM] | [0.15; 0.31] | [0.62; 1.88] | [0.80; 1.55] |
| Kᵢ [nM] | — | [80.2; 148.2] | [69.5; 133.3] |

Kinetic values for the hydrolysis of chromozym t-PA by fXa and the inhibition of fXa by fraction B11SI in the absent and present of heparin calculated using GraphPad Prism version 5.00.

### Example 3

### Comparison of Fraction B11SI and H11p1 by nESI

According to the MS/MS results in example 1 fraction B11SI did not differ from the other tested fractions in the presence of proteins. To examine the reason for the unique ability of this fraction to inhibit fXa it was compared to a fraction that showed no inhibiting effect towards fXa, in this case fraction H11p1. The two fractions were compared by nESI on the intact protein sample. Cytochrome C (about 12,000 Da) was included as a standard. The results were deconvoluted using the Maximum entropy algorithm.

The highest peak in both fractions besides the standard at about 12,000 Da, were at 20,830 and 20,819 for B11SI and H11p1, respectively. According to the MS/MS results these masses were equal to KPI A-k1 N-terminal variant 3 and KPI B-k1 N-terminal variant 5, respectively. Besides the large peak in the results from fraction B1151 peaks are also seen at 20,702, 20,917 and 21,128 Da corresponding to KPI A-k1 N-terminal variant 4, 2 and 1, respectively. Therefore, it is most likely that KPI A-k1 is the fXa inhibitor since fraction B11SI was the best inhibitor of this coagulation factor.

### Conclusion (Example 1, 2 and 3)

Factor Xa was efficiently and specifically inhibited by fraction B11SI. Kinetic values were determined and K was found to be around 100 nM. According to the nESI results the major inhibitor in fraction B11SI was KPI A-k1 (SEQ ID NO:1).

### Example 4

### Coagulation Assay with PIfXa in Both Human and Porcine Plasma

### Method

To make an inhibition profile of PIfXa, 100 μL plasma (human or porcine) was added to a 96 well microtiter plate and mixed with 68-x μL 25 mM hepes buffer, pH 7.4, x μL KPI A-k1 (the volume was dependent on the concentration, and the desired concentration of PIfXa) and finally 32 μL 0.05 M CaCl₂ to initiate the coagulation. The turbidity of the samples was followed by measuring the absorbance at 590 nm every 2 minutes for 3 hours. The curve represents a mean of three replicates.

### Results

With both the porcine and the human plasma a dose dependent delay of coagulation was observed in the presence of PIfXa. In both cases the plasma coagulation is not fully inhibited but prolonged. When 65 μg/mL PIfXa was added to the porcine plasma coagulation was prolonged for 40 min, with the same amount added to human plasma the coagulation time was prolonged for app. 110 min. The difference in the coagulation time between the human and the porcine plasma is due to the fact that porcine plasma is hyper-coagulable compared with human plasma [Olsen et al., 1999] and that the pigs prior to slathering have been put under a lot of stress which also is known to decrease the blood coagulation time [Känel et al., 2001].

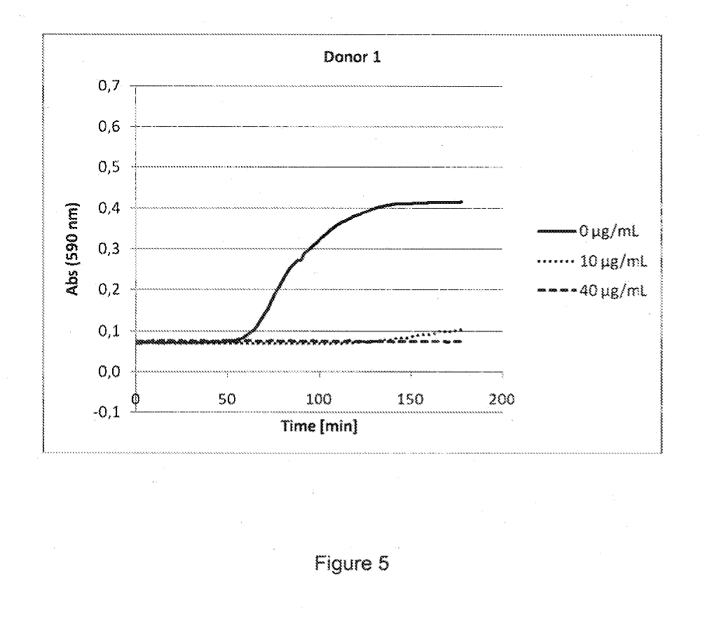

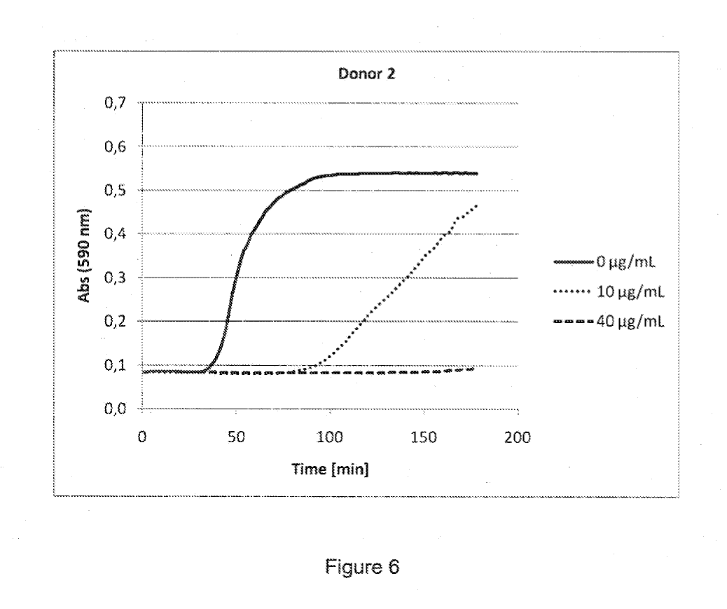

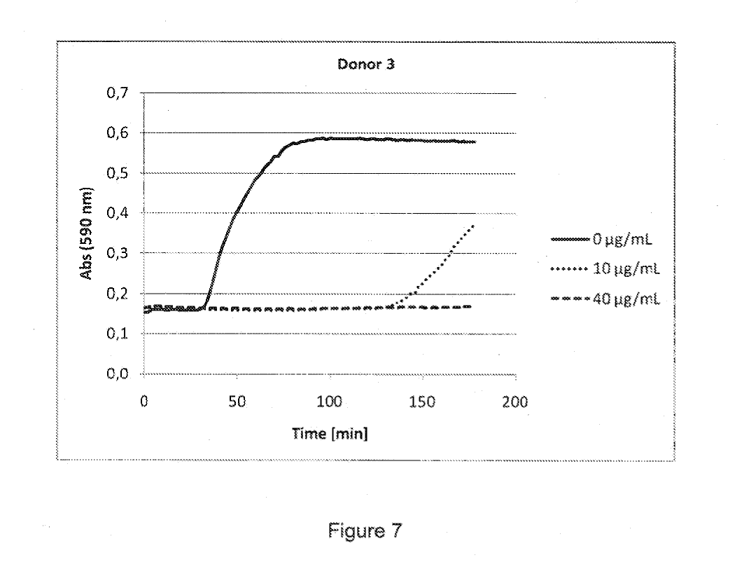

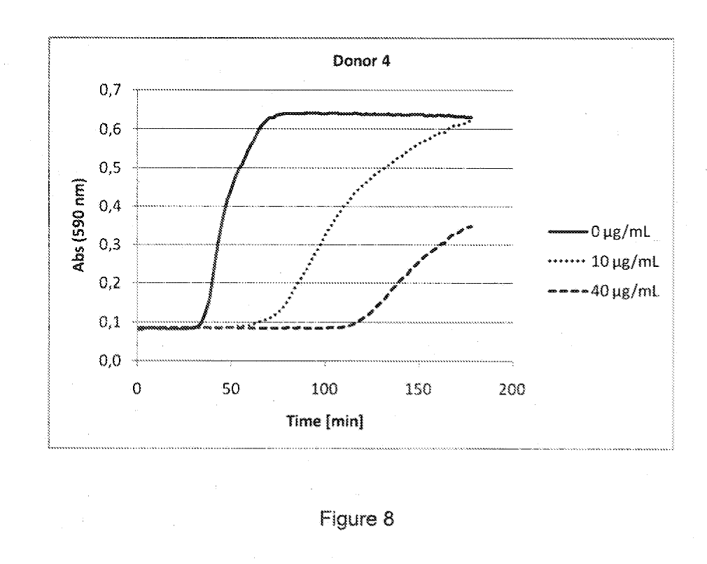

A variation in plasma coagulation among different human donors was observed (FIG. 5-8). The figures show the variation between four different donors. The plasma coagulation without added inhibitor varied from 45 to 55 min. 40 μg/mL showed full effect in three out of the four donors beyond the 180 min the experiment lasted, while a dose of 10 μg/mL gave different effect in all four donors.

### Example 5

### Amidolytic Activity Assay

### Method

The proteins ability to inhibit fXa was analysed using an amidolytic activity assay. All amidolytic activity assays were carried out in triplicates in 50 mM tris, 100 mM NaCl, 10 mM CaCl₂, 0.1% PEG 6000, pH 8 at 25° C. with chromozym t-PA as substrate [Hopfner et al., 1997], [Soejima et al., 2001], [Hayakwa et al., 2000].

fXa, Thrombin and fVIIa Inhibition:

Buffer, enzyme (25 nM fXa and 100 nM fVIIa and thrombin) and inhibitor (65 μg/mL) were added to a 96 well plate and incubated for 10 min at room temperature. The reactions were started by addition of 0.5 mM chromozym t-PA and the change in absorption was measured at 405 nm every 30 sec in an ELISA reader. Absorbances were converted into p-nitroaniline formation (mM) using a molar absorption coefficient of 9900 M⁻¹ cm⁻¹ [Tans et. al, 1987] and a light path length of 0.285 cm. The light path length was calculated from the volume of sample (100 μL), the total volume of the well (382 μL) and the height of the well (10.9 mm) [Greiner Bio One, 2004].

### Results

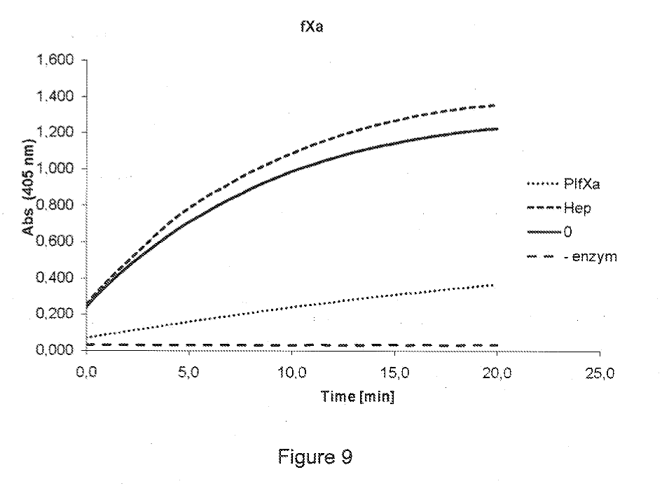

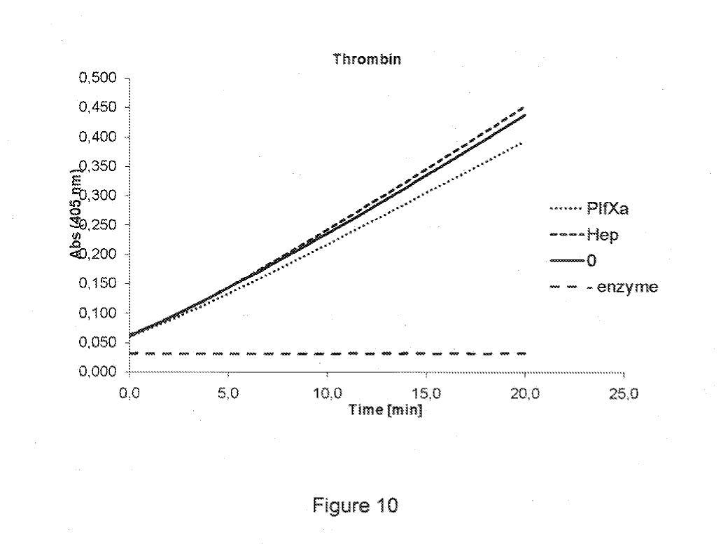

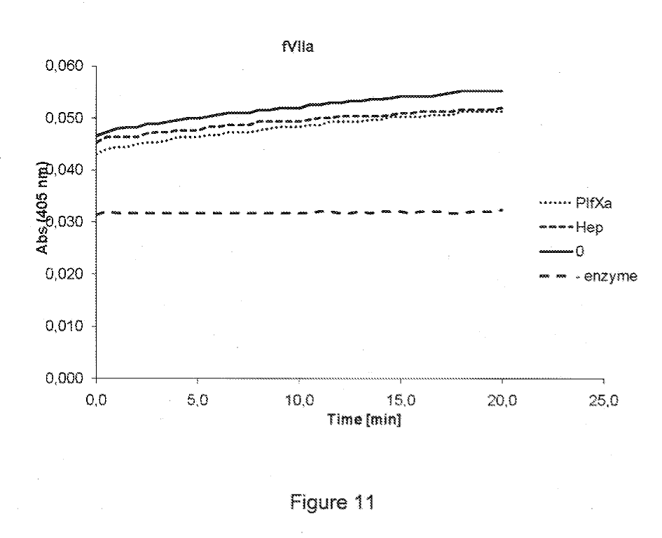

FIG. 9-11 show that PIfXa only has an inhibitory effect on fXa among the enzymes tested, since neither thrombin nor fVIIa was inhibited by any of the tested fractions.

### Example 6

### Reversible Inhibition of fXa

### Method

100 μg/mL PIfXa, 25 nM fXa is mixed with amidolytic activity buffer and incubated for 10 min where after 0.5 mM chromozym t-PA is added and the absorbance is measured every 30 sec. at 405 nm. After 5 min antibody specific for PIfXa is added at 1:1, 1:2.5, 1:5 and 1:10 of the total sample volume and assay is continued for another 25 min.

### Results

The advantage with a protein based inhibitor is that it is possible to raise antibodies towards the protein and use it to reverse the inhibitory effect.

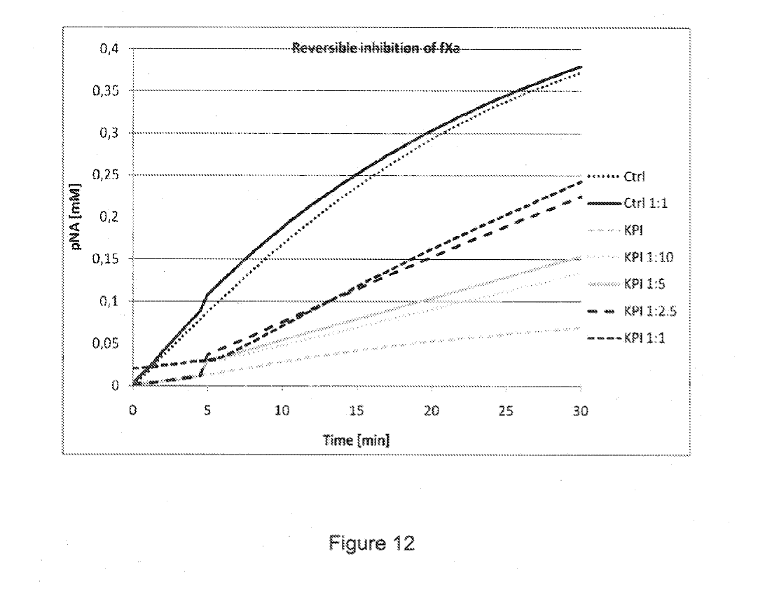

In FIG. 12 different amounts of antibody was added to the fXa, PIfXa solution 5 min into the amidolytic activity assay. Table 5 shows the reaction rate before and after addition of antibody.

TABLE 5
The rates from FIG. 12 before and after addition of antibody.

| | Before [μM/min] | After [μM/min] |
|---|---|---|
| Ctrl | 19.2 | — |
| PlfXa | 2.0 | — |
| PlfXa 1:1 | 2.1 | 8.8 |
| PlfXa 1:2.5 | 2.1 | 7.6 |
| PlfXa 1:5 | 2.2 | 5.0 |
| PlfXa 1:10 | 1.4 | 4.3 |

Both Table 5 and FIG. 12 show that the addition of antibody clearly affects the reaction rate. But with the highest concentration of antibody tested, however, the reaction is still not fully reversed. Most likely, complete reversal of inhibition is not attainable with a polyclonal antibody, because antibodies specific for the binding interface does not constitute enough of the polyclonal antibody that can be physically added to the assay to fully saturate binding sites.

### Example 7

### Activated Partial Thromboplastin Time (aPTT) Assay

### Method

Plasma provided from the coagulation lab at Aalborg Hospital was mixed with a small volume of KPI A-k1 to obtain concentrations from 0-300 μg/mL of protein in the plasma. 100 μL of this solution was then transferred to a cuvette and a magnetic ball was added. The cuvette was placed in a coagulameter, where it was stirred. 100 μL of Pathrombin* SL Reagent was added and the sample was incubated at 37° C. for 5 minutes. To initiate the coagulation 100 μL of 20 mM CaCl2 was added, and the time needed to form a fibrin clot was measured. The coagulation time was, in this assay, defined as the time before the ball was stopped by the viscosity of the coagulated plasma. To compare the effect of the inhibitor on the plasma coagulation with other anticoagulants, the assay was performed with Fragmin, which is a low molecular weight heparin (LMWH), and fondaparinux. Fragmin and fondaparinux were added to the plasma to obtain concentrations of 0-2 IU/mL and 0-2.5 IU/mL respectively. These concentrations were chosen on the basis of the therapeutic doses of the two anticoagulants [Beatrice et al., 2001; Smogorzewska et al., 2006]. All assays were carried out in triplicates.

### Results

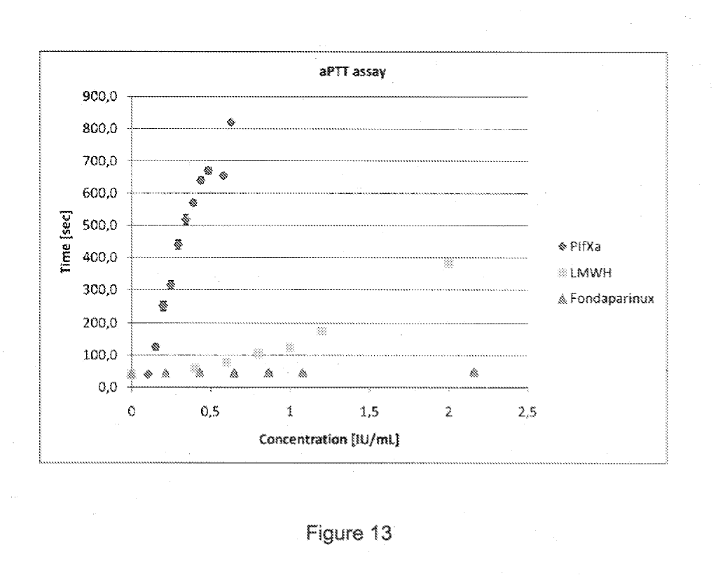

Fondaparinux, a heparin based fXa inhibitor and low-molecular weight heparin (LMWH) showed no effect in the aPPT assay, according to the current literature the missing effect is due to the fact that the aPTT assay is dependent on inhibitory activity against thrombin rather than fXa [LinKins et al., 2002]. This claim does not correspond to the data found in this project, where although no inhibitory effect is seen towards thrombin (FIG. 12) an effect was clearly observed in the aPTT assay (FIG. 13).

Another method for monitoring the inhibition effect of PIfXa, the COAMATIC Heparin assay, was used, based on a standard curve generated from LMWH. In this assay 200 μg/mL PIfXa shows similar effect as 0.5-0.8 IU/mL of LMWH which is the usual therapeutic dose.

### Example 8

### Whole Blood Coagulation Assay

### Method

Drops of the purified inhibitor were placed on a glass plate in various concentrations (0-60 μg/mL). Blood was drawn from a finger with a 21 gage needle, 10 μL was placed in the inhibitor and mixed. Coagulation time was defined as the time from when the drop of blood was placed until small thread like structures could be pulled from the solution with a pipette tip. The assay was performed in duplicates.

### Results

The table shows the coagulation time for whole blood at different concentrations of PIfXa

TABLE 6
Coagulation time for whole blood at PlfXa
concentrations from 0-60 μg/mL

| Conc. [μg/mL] | Coagulation time [s] | Std. deviation |
|---|---|---|
| 0 | 307.5 | 31.8 |
| 5 | 376.5 | 21.9 |
| 15 | 459.5 | 4.9 |
| 30 | 558.5 | 125.2 |
| 60 | >1320 | — |

This very simple assay also showed that the coagulation time was prolonged with higher concentrations of PIfXa.

### Example 9

### Both fX and fXa Inhibitor?

### Method

0.8 μM fX was incubated for 10 min. at room temperature with 2.4 μM PIfXa in assay buffer (10 mM Hepes, 100 mM NaCl, 5 mM CaCl₂, 0.1% PEG, pH 7.5) where after 0.8 μM RVV was added and incubated for 45 min at room temperature. The solution was mixed with loading buffer and run on a 12% SDS-gel under both reducing and non-reducing conditions. As controls a solution of fX and RVV, fX and KPI, and RVV and KPI was used.

### Results

To se whether PIfXa was able to inhibit both the pro-enzyme and activated fX an SDS PAGE gel based assay was developed.

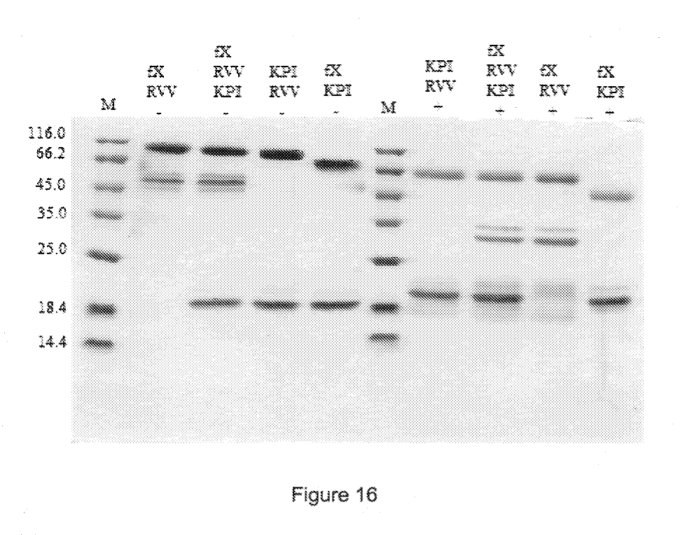

As can be seen on the gel (FIG. 16) fX was activated, to the same extend regardless of the presence of PIfXa. This indicates that PIfXa does not influence the activation of fX but only the activated enzyme.

### Example 10

### Pilot Animal Experiment

### Method

4 different samples with PIFXa were prepared with a concentration of 0, 0.2, 0.75 and 1.3 mg. To each sample bovine albumin was added to a total protein concentration of 1.3 mg. The 4 concentrations were conducted in triplicates, 200 IU/kg heparin was given to 4 rats and 4 rats did not receive anything.

The rats were acclimatized for a couple of days prior to the experiment. One the day of the experiment the rats were sedated with 5% isofluran in an induction chamber. They were weight and placed on a heating mat. The sample (500 μL) was injected over 10 sec by an intravenous catheter placed in the tail vein. After 10 min pause, 2 mm of the tips of the tail was cut off and the tail was placed in 50 mL heated saline. The bleeding time was recorded for 30 min along with the temperature of the saline and the respiratory frequency. After 30 min SDS were added to the saline to at final concentration of 2% and the rats were killed by a pull of the neck.

### Results

The results from the bleeding time showed that there was an inhibitory effect from PIfXa and that it was comparable to that of heparin. The three tested concentrations of PIfXa gave app. the same result indicating that the maximum effect was already reached at the lowest concentration.

The absorbance measurements showed an initial increase in haemoglobin release with small amounts of PIfXa, but this effect was reversed with increasing amounts of PIfXa. This, seemingly contradictory observation, can be an effect of platelet clots in the tail. Since PIfXa, in contrast to heparin, only inhibits fXa the primary response of the blood coagulation, namely the platelet plug formation is free to form. This could mean that although no fibrin clot is being formed because of the inhibited fXa, a platelet plug is still being formed in the tail. This plug could prevent the blood cells from flowing out of the tail but still allow plasma flow which means that bleeding is still detected (in bleeding time), but because haemoglobin is trapped in the much larger red blood cells, it is trapped in the plug. Heparin is a much less specific molecule inhibiting both plasma and platelet aggregation via interacting with thrombin, which can act as a platelet activator and von Willebrand factor, which participates in platelet aggregation.

### Example 11

### Animal Experiment

### Method

The experiment was preformed as in the pilot experiment but the doses of PIfXa were 0, 0.063, 0.125, 0.25 and 0.5 mg. As controls heparin and animal who did not receive anything was used. Seven rats was used in each group (n=7).

### Results

The experiments from both the pilot the animal experiment were divided into three different groups, low (0.063 and 0.125 mg), medium (0.2 and 0.25 mg) and high (0.5, 0.75 and 1.3 mg) dose. As with the pilot experiment it was observed that a maximum bleeding time was reached with the low dose.

The haemoglobin amount in the samples from the rats receiving PIfXa was as in the pilot experiment much lower than in the samples with heparin. This is likely due to a formation of a platelet plug formation as discussed under the results for the pilot experiments.

### Example 12

### Expression of PIfXa in Nicotiana benthamiana Leaves

### Method

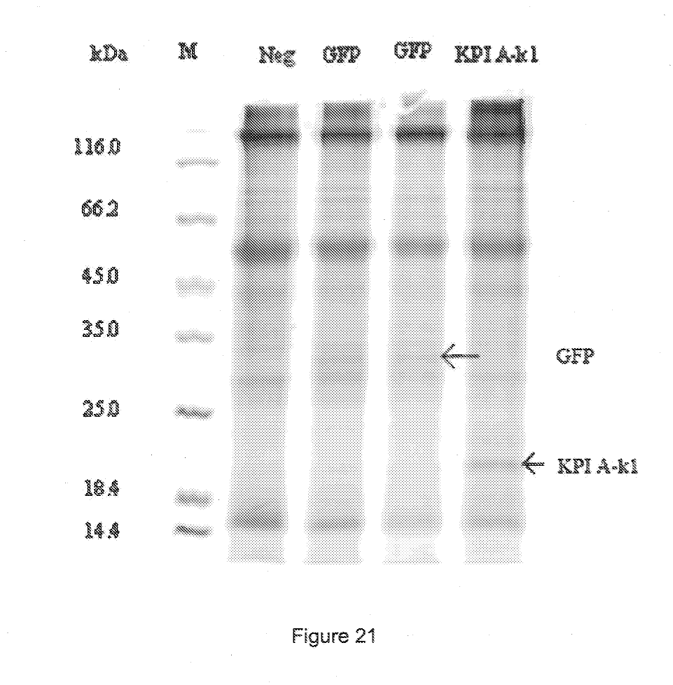

The gene coding for PIfXa was cloned into the pCAMBIA2300 vector in a PacI cassette. pCAMBIA2300-GFP vector containing the gene coding for GFP (green fluorescence protein) and pCAMBIA2300 without any insert was used as positive and negative control, respectively. The vectors were transferred into agrobacterium which were infiltrated into Nicotiana benthamiana leafs. After four days the proteins were extracted from the leaves and the expression was examined on a SDS PAGE gel (FIG. 21).

### Results

GFP was expressed in the positive controls and PIfXa was expressed as obvious by the presence of an additional band on the SDS PAGE gel at the expected size (21 kDa). Furthermore, the identity was confirmed by sequencing of peptide tags by LC MS/MS.

### REFERENCES

Beatrice, K. H., Thomsen, A. R., Moriarty, H. T. (2001) “A comparison of the sensitivity of APTT reagents to the effects of enoxaparin, A Low-molecular weight heparin” Pathology, Vol. 33, pp. 347-352
Greiner Bio One (2004) “Customer drawing, PS Mircoplate, 96 well, F-bottom” www.greinerbioone.com/en/row/articles/catalogue/article/73_{—}11/13243/
Hayakawa, Y., Hayashi, T., Lee, J., Ozawa, T. and Sakuragawa, N. (2000) “Activation of Heparin Cofactor II by Calcium Spirulan” The Journal of Biological Chemistry, Vol. 275, No 15, pp. 11379-11382.
Hopfner, K., Brandstetter, H., Karcher, A., Kopetzki, E., Huber, R., Engh, R. A. and Bode, W. (1997) “Converting blood coagulation factor IXa into factor Xa: dramatic increase in amidolytic activity identifies important active site determinants” The EMBO Journal, Vol. 16, No. 22, pp. 6626-6635.
Känel, R. V., Mills, P. J., Fainman, C. and Dimsdale, J. E (2001) “Effects of Psychological Stress and Psychiatric Disorders on Blood Coagulation and Fibrinolysis: A Biobehavioral Pathway to Coronary Artery Disease?” Psychosomatic Medicine, Vol. 63, pp. 531-544.
Linkins, L., Julian, J. A., Rischke, J., Hirsh J. and Weitz, J. I. (2002) “In vitro comparison of the effect of heparin, enoxapin and fondaparinux on tests of coagulation” Thrombosis Reasearch, Vol 107 pp 241-244.
Olsen, A. K., Hansen, A. K., Jespersen, J., Marckmann, P. and Bladbjerg, E. M (1999) “The pig as a model in blood coagulation and fibrinolysis research” Scand. J. Lab. Anim., Vol 26. No. 4 pp 214-224.
Smogorzewska, A., Brandt, J. T., Chandler, W. L., Cunningham, M. T., Hayes, T. E., Olson, J. D., Kottke-Marchant, K., Van Cott, E. M. (2006) “Effect of Fondaparinux on Coagulation Assays Results of College of American Pathologists Proficincy Testing” Archives of Pathology and Laboratory Medicine, Vol. 130, no. 11, pp. 1605-1611
Soejima, K., Mizuguchi, J., Yuguchi, M., Nakagaki, T., Higashi, S. and Iwanaga, S. (2001) “Factor VIIa Modified in the 170 Loop Shows Enhanced Catalytic Activity but Does Not Change the Zymogen-like Property”, Journal of Biological Chemistry, Vol. 276, No. 20, pp. 17229-17235.
Tans, G., Janssen-Claessen, T., Rosing, J. and Griffin, J. H. (1987). “Studies on the effect of serine protease inhibitors on activated contact factors. Application in amidolytic assays for factor XIIa, plasma kallikrein and factor XIa” Eur. J. Biochem, Vol. 164, pp. 637-642.

1. A polypeptide or a composition comprising a polypeptide, wherein said polypeptide comprises or consists of SEQ ID NO:1, or a polypeptide having at least 80% sequence identity with SEQ ID NO: 1, or a polypeptide fragment of SEQ ID NO: 1.

2. A pharmaceutical composition comprising
a. a polypeptide comprising or consisting of SEQ ID NO:1, or a polypeptide having at least 80% sequence identity with SEQ ID NO:1, or a polypeptide fragment of SEQ ID NO:1, and
b. a pharmaceutical acceptable carrier.

3. The composition according to claim 1, wherein the polypeptide has at least 80% sequence identity, such as at least 81% sequence identity, e.g. at least 82% sequence identity, such as at least 83% sequence identity, e.g. at least 84% sequence identity, such as at least 85% sequence identity, e.g. at least 86% sequence identity, such as at least 87% sequence identity, e.g. at least 88% sequence identity, such as at least 89% sequence identity, e.g. at least 90% sequence identity, such as at least 91% sequence identity, e.g. at least 92% sequence identity, such as at least 93% sequence identity, e.g. at least 94% sequence identity, such as at least 95% sequence identity, e.g. at least 96% sequence identity, such as at least 97% sequence identity, e.g. at least 98% sequence identity, such as at least 99% sequence identity, e.g. at least 99.5% sequence identity to SEQ ID NO:1, or a fragment of SEQ ID NO:1.

4. The composition according to claim 1 wherein said polypeptide or fragment thereof is capable of inhibiting the activity of a protease of the blood clotting cascade.

5. A method of treating a disorder of the heart or blood vessels, the method comprising administering:
a polypeptide or a composition comprising a polypeptide, wherein said polypeptide comprises or consists of SEQ ID NO:1, or a polypeptide having at least 80% sequence identity with SEQ ID NO: 1, or a polypeptide fragment of SEQ ID NO: 1.

6. The method of claim 5, wherein the disorder comprises, atrial fibrillation and cardioversion.

7. The method of claim 5, wherein the disorder comprises, peripheral arterial occlusion caused by primary or recurrent thrombus formation, embolism or atherosclerosis.

8. The method of claim 5, wherein the disorder comprises, deep-vein thrombosis and pulmonary embolism.

9. The method of claim 5, wherein the disorder comprises, blood clotting in extracorporal circuits during cardiopulmonary bypass and hemodialysis.

10. The method of claim 5, wherein the disorder comprises, blood clotting during angioplastic procedures.

11. The method of claim 5, wherein the disorder comprises, blood clotting in connection with prosthetic heart valve replacement.

12. The method of claim 5, wherein the disorder comprises, blood clotting due to diseases affecting the heart valves comprising infected valves (bacterial endocarditis), rheumatic mitral valve disease, mitral stenosis, mitral valve prolapse, mitral annular calcification and isolated aortic valve disease.

13. The method of claim 5, wherein the disorder comprises, blood clotting in patients with thrombophilia syndromes including antiphospholipid syndrome, Factor V Leiden, prothrombin mutation/factor II mutation, high homocysteine levels due to MTHFR mutation or vitamin deficiency (vitamins B6, B12 and folic acid), renal loss of antithrombin, plasminogen and fibrinolysis disorders, paroxysmal nocturnal hemoglobinuria, protein C deficiency, protein S deficiency, and antithrombin III deficiency.

14. The method of claim 5, wherein the disorder comprises, blood clotting following coagulation management warranted by the presence of conditions that increase the risk of bleeding comprising Hemophilia A, Hemophilia B, Hemophilia C, Von Willebrand disease, major blood loss, Glanzmann's thrombasthenia, Bernard-Soulier syndrome, gray platelet syndrome and delta storage pool deficiency.

15. The method of claim 5, wherein the disorder comprises, blood clotting due to decreased platelet number caused by insufficient production (e.g. in myelodysplastic syndrome or other bone marrow disorders), destruction by the immune system (immune thrombocytopenic purpura/ITP), and consumption due to various causes (thrombotic thrombocytopenic purpura/TTP, hemolytic-uremic syndrome/HUS, paroxysmal nocturnal hemoglobinuria/PNH, disseminated intravascular coagulation/DIC, heparin-induced thrombocytopenia/HIT).

16. The method of claim 5, wherein the disorder comprises, blood clotting in individuals who do not tolerate other medicaments targeting blood clotting on the market.

17. The method of claim 5, wherein the disorder comprises, blood clotting in individuals that are immobilized for any reason, patients that suffer from critical limb ischemia or have had a limb amputated.

18. The method of claim 5, wherein the disorder comprises, blood clotting in patients receiving any form for chemotherapeutics.

19. The method of claim 5, wherein the disorder comprises, blood clotting in patients at high risk for developing blood clots.

20. A method for inhibiting Factor X activity, comprising the steps of administering a polypeptide according to claim 1 to an individual and inhibiting Factor X in said individual.

21. A method for reversing the inhibition of Factor X by administration of one or more antibodies to a site wherein a polypeptide according to claim 1 inhibits factor X, binding of said one or more antibodies to said polypeptide, at least partly dissociating the polypeptide from the Factor X, thereby reversing the inhibition of Factor X activity.

22. A method for collecting blood samples, comprising the step of collecting a blood sample and storing said blood sample in a container coated with a polypeptide as defined in claim 1.

23. A method comprising coating medicotechnological device intended to be in contact of at least one mammalian body fluid with the polypeptide of claim 1.

24. A medico-technological device intended to be in contact with at least one mammalian body fluid coated with a polypeptide or a composition as defined in claim 1.

25. The method of claim 5, wherein the disorder comprises stable angina pectoris, unstable angina pectoris, myocardial ischemia, myocardial infarction, dilated cardiomyopathy, hypertropic cardiomyopathy, congestive heart failure, or cardiac failure.


SEQUENCE LISTING (2 sequences)

SEQ ID NO: 1 - protein, 220 aa, Solanum tuberosum
MMKCLFFLCLCLFPILVFSSTFTSQNPINLPSESPLPKPVLDTNGKELNPNLSYRIISTYWGALGGDVYLGKSPNSDAPCPDGVFRYNSDVGPSGTPVRFIPLSTNIFEDQLLNIQFNIPTPKLCVSYTIWKVGNINAPLRTMLLETGGTIGQADSSYFKIVKSSNFGYNLLYCPITRHFLCPFCRDDNFCAKVGVVIQNGKRRLALVNENPLDVLFQEV

SEQ ID NO: 2 - DNA, 835 bp, Solanum tuberosum
aatcaatatgatgaagtgtttatttttcttatgtttgtgtttgtttcccattttggtgttttcatcaactttcacttcccaaaatcccatcaacctacccagtgaatctcctctacctaagccggtacttgacacaaatggtaaagaactcaatcctaatttgagttatcgcattatttccacttattggggtgccttaggtggtgatgtgtaccttggaaagtccccaaattcagatgccccttgtccagatggcgtattccgttacaattccgatgttggacctagcggtacacccgttagattcattcctttatctacaaatatctttgaagatcaactacttaacatacaattcaatattcctacaccgaaattatgtgttagttatacaatttggaaagtcggtaatataaatgcacctctaaggacgatgttgttggagactggaggaaccatagggcaagcagatagcagctatttcaagattgttaaatcatcaaattttggttacaacttattgtattgccctattactcgccattttctttgtccattttgtcgtgatgataacttctgtgcaaaagtgggtgtagttattcaaaatggaaaaaggcgtttggctcttgtcaacgaaaatcctcttgatgtcttattccaggaagtctagtaacaaataatgcctgcagctagactatactatgttttagcctgctggttagctactatgttatgttgtaaattaaaataaacacctgctaaggtatatctatattttagcatggatttcttaataaattgtctttccttatcgtttaaaaaaaaaaaaaaaaaa